(19) United States
(12) Patent Application Publication
(10) Pub. No.: US 2014/0235479 A1
(43) Pub. Date: Aug. 21, 2014

(54) SIGNATURES AND PCDETERMINANTS ASSOCIATED WITH PROSTATE CANCER AND METHODS OF USE THEREOF

(71) Applicants: Ronald A. Depinho, Houston, TX (US); Zhihu Ding, Brookline, MA (US); Lynda Chin, Houston, TX (US)

(72) Inventors: Ronald A. Depinho, Houston, TX (US); Zhihu Ding, Brookline, MA (US); Lynda Chin, Houston, TX (US)

(21) Appl. No.: 14/174,072

(22) Filed: Feb. 6, 2014

Related U.S. Application Data
(63) Continuation of application No. 13/054,468, filed on Jun. 2, 2011, now abandoned.
(60) Provisional application No. 61/081,286, filed on Jul. 16, 2008.

(51) Int. Cl. G01N 33/574; C12Q 1/68

(52) U.S. Cl. CPC G01N 33/57434; C12Q 1/6886
USPC 506/9; 435/7.92; 436/501; 506/16; 435/7.4; 435/6.12; 435/7.1; 435/7.23

(57) ABSTRACT

The present invention provides methods of detecting cancer using biomarkers.

### RELATED APPLICATION

This application claims the benefit of U.S. Ser. No. 61/081,286, filed Jul. 16, 2008, the contents of which are incorporated herein by reference in its entirety.

### FIELD OF THE INVENTION

The present invention relates generally to the identification of biological signatures associated with and genetic PCDETERMINANTS effecting cancer metastasis and methods of using such biological signatures and PCDETERMINANTS in the screening, prevention, diagnosis, therapy, monitoring, and prognosis of cancer. The invention further relates to a genetically engineered mouse model of metastatic prostate cancer.

### BACKGROUND OF THE INVENTION

Prostate cancer (PCA) is the most frequent male cancer and a leading cause of cancer death in US. Most elderly men harbor prostatic neoplasia with the vast majority of cases remaining localized and indolent without need for therapeutic intervention. There are however a subset of early stage PCAs “hardwired” for aggressive malignant behavior stage and, if left untreated, will spread beyond the prostate and progress relentlessly to metastatic disease and ultimately death. The current inability to accurately distinguish indolent and aggressive disease has subjected many men with potentially indolent disease to unnecessary therapeutic interventions with high morbidity.

Current methods of stratifying tumors to predict outcome are based on clinicopathological factors including Gleason grade, PSA, and tumor stage. Although these formulae are helpful, they do not fully predict outcome and importantly are not reliably linked to the most meaningful clinical endpoints of risk of metastatic disease and PCA-specific death. This unmet medical need has fueled efforts to define the genetic and biological bases of PCA progression with the goals of identifying biomarkers capable to assigning progression risk and providing opportunities for targeted interventional therapies. Genetic studies of human PCA has identified a number of signature events including PTEN tumor suppressor inactivation and ETS family translocation and dysregulation, as well as many other important genetic and/or epigenetic alterations including Nkx3.1, c-Myc and SPINK. Global molecular analyses have also identified an array of potential recurrence/metastasis biomarkers, such as ECAD, AIPC, Pim-1 Kinase, hepsin, AMACR, and EZH2. However, the intense heterogeneity of human PCA has limited the utility of single biomarkers in the clinical setting, thus prompting more comprehensive transcriptional profiling studies to define prognostic multi-gene biomarker panels or signatures. These predictive signatures appear to be more robust; however their clinical utility has remained uncertain due to the inherent noise and context-specific nature of transcriptional networks and the extreme instability of cancer genomes with myriad bystander genetic and epigenetic events producing significant disease heterogeneity. These factors have conspired to impede the identification of biomarkers capable of accurately assigning risk of disease progression. Accordingly, a need exists for more accurate models of human cancer that can be used together with complex human datasets to identify robust biomarkers that can be used to predict the occurrence and the behavior of cancer, particularly at an early stage.

### SUMMARY OF THE INVENTION

The present invention relates in part to the discovery that certain biological markers (referred to herein as “PCDETERMINANTS”), such as proteins, nucleic acids, polymorphisms, metabolites, and other analytes, as well as certain physiological conditions and states, are present or altered in early stage cancers which endow these neoplasm with an increased risk of recurrence and progression to metastatic cancer. The cancer is for example prostate cancer or breast cancer.

Accordingly, in one aspect the invention provides a method with a predetermined level of predictability for assessing a risk of development of metastatic cancer in a subject. Risk of developing metastatic prostate cancer is determined by measuring the level of a PCDETERMINANT in a sample from the subject. An increased risk of developing metastatic cancer in the subject is determined by measuring a clinically significant alteration in the level of the PCDETERMINANT in the sample. Alternatively, an increased risk of developing metastatic cancer in the subject is determined by comparing the level of the effective amount PCDETERMINANT to a reference value. In some aspects the reference value is an index.

In another aspect, the invention provides a method with a predetermined level of predictability for assessing the progression of a tumor in a subject by detecting the level of PCDETERMINANTS in a first sample from the subject at a first period of time, detecting the level of PCDETERMINANTS in a second sample from the subject at a second period of time and comparing the level of the PCDETERMINANTS detected to a reference value. In some aspects the first sample is taken from the subject prior to being treated for the tumor and the second sample is taken from the subject after being treated for the tumor.

In a further aspect, the invention provides a method with a predetermined level of predictability for monitoring the effectiveness of treatment or selecting a treatment regimen for metastatic cancer by detecting the level of PCDETERMINANTS in a first sample from the subject at a first period of time and optionally detecting the level of an effective amount of PCDETERMINANTS in a second sample from the subject at a second period of time. The level of the effective amount of PCDETERMINANTS detected at the first period of time is compared to the level detected at the second period of time or alternatively a reference value. Effectiveness of treatment is monitored by a change in the level of the effective amount of PCDETERMINANTS from the subject.

A PCDETERMINANT includes for example DETERMINAT 1-372 described herein. One, two, three, four, five, ten or more PCDETERMINANTS are measured. In some embodiments least two PCDETERMINANTS selected from the PCDETERMINANTS listed on Table 2, 3, 4, 5, 6, or 7 are measured. Preferably, PTEN, SMAD4, cyclin D1 and SPP1 are measured. Optionally, the methods of the invention further include measuring at least one standard parameters associated with a tumor. A standard parameter is for example Gleason Score.

The level of a PCDETERMINANT is measured electrophoretically or immunochemically. For example the level of the PCDETERMINANT is detected by radioimmunoassay, immunofluorescence assay or by an enzyme-linked immunosorbent assay. Optionally, the PCDETERMINANT is detected using non-invasive imaging technology.

The subject has a primary tumor, a recurrent tumor, or metastatic cancer. In some aspects the sample is taken for a subject that has previously been treated for the tumor. Alternatively, the sample is taken from the subject prior to being treated for the tumor. The sample is a tumor biopsy such as a core biopsy, an excisional tissue biopsy or an incisional tissue biopsy. The sample is blood or a circulating tumor cell in a biological fluid.

Also included in the invention is metastatic prostate cancer reference expression profile containing a pattern of marker levels of an effective amount of two or more markers selected from PCDETERMINANTS 1-372. Preferably, the profile contains a pattern of marker levels of the PCDETERMINANTS listed on any one of Tables 1A, 1B, 2, 3, 4, 5, 6, or 7. Also included is a machine readable media containing one or more metastatic tumor reference expression profiles and optionally, additional test results and subject information. In another aspect the invention provides a kit comprising a plurality of PCDETERMINANT detection reagents that detect the corresponding PCDETERMINANTS. For example, the kit includes PTEN, SMAD4, cyclin D1 and SPP1 detection reagents. The detection reagent is for example antibodies or fragments thereof, oligonucleotides or aptamers.

In a further aspect the invention provides a PCDETERMINANT panel containing one or more PCDETERMINANTS that are indicative of a physiological or biochemical pathway associated metastasis or the progression of a tumor. The physiological or biochemical pathway includes for example, P13K, RAC-RHO, FAK, and RAS signaling pathways.

In yet another aspect, the invention provides a method of identifying a biomarker that is prognostic for a disease by identifying one or more genes that are differentially expressed in the disease compared to a control to produce a gene target list; and identifying one or more genes on the target list that is associated with a functional aspect of the progression of the disease. The functional aspect is for example, cell migration, angiogenesis, distal colonization, extracellular matrix degradation or anoikis. Optionally, the method includes identifying one or more genes on the gene target list that comprise an evolutionarily conserved change to produce a second gene target list. The disease is for example cancer such as invasive or metastatic cancer.

Compounds that modulates the activity or expression of a PCDETERMINANT are identified by providing a cell expressing the PCDETERMINANT, contacting (e.g., in vivo, ex vivo or in vitro) the cell with a composition comprising a candidate compound; and determining whether the substance alters the expression of activity of the PCDETERMINANT. If the alteration observed in the presence of the compound is not observed when the cell is contacted with a composition devoid of the compound, the compound identified modulates the activity or expression of a PCDETERMINANT.

Cancer is treated in a subject by administering to the subject a compound that modulates the activity or expression of a PCDETERMINANT or by administering to the subject an agent that modulates the activity or expression of a compound that is modulated by a PCDETERMINANT.

Cancer is treated by providing a subject whose cancer cells have clinically significant alteration in the level of the two or more of PCDETERMINANTS 1-372 and treating the subject with adjuvant therapy in addition to surgery or radiation. The alteration in the level of the PCDETERMINANTS indicates an increased risk of cancer recurrence or developing metastatic cancer in the subject. Additionally, prostate cancer is treated in a subject in need thereof by obtaining information on the expression levels of PTEN, SMAD4, CYCLIN D1 and SPP1 in a sample from prostate cancer tissue in the subject; and administering an SPP1 inhibitor, a CD44 inhibitor, or both. The subject is one identified as being at risk for recurrence of prostate cancer or development of metastatic cancer based on expression levels of PTEN, SMAD4, CYCLIN D1 and SPP1.

In one aspect the invention provide a method of selecting a tumor patient in need of adjuvant treatment by assessing the risk of metastasis in the patient by measuring an effective amount of PC DETERMINANTS where a clinically significant alteration two or more PCDETERMINANTS in a tumor sample from the patient indicates that the patient is in need of adjuvant treatment. For example, the methods describes herein are useful in determining whether as particular subject is suitable for a clinical trial.

In a further aspect the invention provides a method of informing a treatment decision for a tumor patient by obtaining information on an effective amount of PCDETERMINANTS in a tumor sample from the patient, and selecting a treatment regimen that prevents or reduces tumor metastasis in the patient if two or more PCDETERMINANTS are altered in a clinically significant manner.

In various embodiments the assessment/monitoring is achieved with a predetermined level of predictability. By predetermined level of predictability is meant that that the method provides an acceptable level of clinical or diagnostic accuracy. Clinical and diagnostic accuracy is determined by methods known in the art, such as by the methods described herein.

The invention further provides a transgenic double knockout mouse whose genome contains genetic modification that enables a homozygous disruption of both the endogenous Pten gene and Smad4 gene in the prostate epithelium. One skilled in the art would recognize that this disruption can be achievement by recombinase-mediated excision of Pten or Smad genes with embedded LoxP site (i.e., the current strain) or by for example mutational knock-in, and RNAi-mediated extinction of these genes either in a germline configuration or in somatic transduction of prostate epithelium in situ or in cell culture followed by reintroduction of these primary cells into the renal capsule or orthotopically. Other engineering strategies are also obvious including chimera formation using targeted ES clones that avoid germline transmission. The transgenic mouse exhibits an increased susceptibility to formation of prostate tumors as compared to a wild type mouse. The mouse also exhibits an increased susceptibility to formation of metastatic prostate cancer as compared to a Pten-only single knockout transgenic mouse. Also includes are cells from the mouse. Preferably, the cells are epithelial cells such as prostate epithelial cells, breast epithelial cells, lung epithelial cells or colon epithelial cells.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 demonstrates that the loss of Pten prostate upregulated the level of p-Smad2/Smad3 and Smad4 expression. (A) Ingenuity Canonical Pathway Analysis of differentially expressed genes between Pten^{pc-/-} mice (3331 probe sets, in blue) were compared to 10 randomly drawn gene sets of equal size. (B) Western blot analysis of AP tissue from each genotype at 15 weeks shows pSmad2/3 level enhanced, Smad4 upregulation, and Id1 induction in Pten^{pc-/-} mice compared to control mice. (C) Immunohistochemistry analysis of 15-week-old APs for Smad4 is performed demonstrating upregulation in Pten^{pc-/-} mice (Panel c) compared to control mice (Panel a). Smad^{pc-/-} mice used as negative control (Panel b). Scale bars, 50 μm. (D, E) Onconmine analysis (http://www.oncomine.org/) of Smad4 expression between human PCA and metastasis. Heatmap of Smad4 differentially expressed in Yu et al prostate expression dataset (D). Boxed plot of Smad4 expression between human PCA and metastasis in Yu et al prostate expression dataset and Dhanasekaran et al (2001) prostate expression dataset (E).

FIG. 2 demonstrate that the loss of Smad4 does not initiate prostate tumors but renders Pten-deficient carcinomas lethal. (A) Histopathological analysis (haematoxylin/eosin staining) of anterior prostates (AP) in WT, Smad4 and Pten single and double mutants at 9 weeks of age reveals normal glands in WT and Smad^{pc-/-} mice but PIN lesions in Pten^{pc-/-} mice and invasion (arrow) in Pten^{pc-/-}; Smad^{pc-/-} mice. Scale bars, 50 μm. (B) Kaplan-Meier overall cumulative survival analysis. A statistically significant decrease in lifespan (P<0.0001) compared with the Pten^{pc-/-} cohort (n=28) was found for the Pten^{pc-/-}; Smad^{pc-/-} cohort (n=26) (asterisk). (C) Gross anatomy of representative WT, Smad^{pc-/-}, Pten^{pc-/-}, and Pten^{pc-/-}; Smad^{pc-/-} anterior prostate or prostate tumor at 22 weeks of age. Scale bars, 0.5 cm.

FIG. 3 demonstrates that the loss of Smad4 enhanced proliferation and circumvented Pten-loss-induced cellular senescence. (A) Histopathological and proliferation analysis of 15-week-old APs demonstrated increase in proliferation at some invasion foci (arrow, panel e) in Pten^{pc-/-}; Smad^{pc-/-} double mutants (panel j). Tunel analysis of 15-week-old APs showed no significant difference in Pten^{pc-/-}; Smad^{pc-/-} double mutants (panel i,j) and Pten^{pc-/-} prostate tumors (panel h). H&E, haematoxylin/eosin. Scale bars, 50 μm. (B) Loss of Smad4 circumvented Pten-loss-induced cellular senescence. β-Gal staining analysis of 15-week-old APs. Scale bars, 100 μm. (C) Quantification of brdu pulse labeling of 15-week-old APs done as in (A,f-j). Representative sections from three mice were counted for each genotype. (D) Quantification of TUNEL assay for apoptosis in the AP at 15 weeks. Representative sections from three mice were counted for each genotype. (E) Quantification of the 3-Gal staining seen on AP sections at 15 weeks done as in (B). Representative sections from three mice were counted for each genotype. Error bars in C-E represent s.d. for a representative experiment performed in triplicate. Asterisk indicates statistical significance between Pten^{pc-/-}; Smad^{pc-/-} double mutants and Pten^{pc-/-} (P<0.05).

FIG. 4 demonstrates that the loss of Smad4 leads to Pten-deficient carcinomas progress to metastasis to lymph nodes and lung with complete penetrance. (A) Metastasis-free survival curve (Kaplan-Meier plot) of prostate cancer. Metastasis foci in lumbar lymph nodes and/or lungs was found only in the Pten^{pc-/-}; Smad^{pc-/-} cohort from 16 to 32 weeks of age. A statistically significant (P<0.0001) compared with the Pten^{pc-/-} cohort (n=25) was found for the Pten^{pc-/-}; Smad^{pc-/-} cohort (n=25) (asterisk) which with complete penetrance of metastasis. (B) Gross anatomy of representative lumbar lymph modes (dashed circle) and lung with metastasis foci (dark arrows). Scale bars, 0.5 cm. (C) H&E stained sections show metastatic prostate cancer cells in the lymph node (dark arrows) and lung. Immunohistochemical analyses show that metastatic cells in lymph node and lung are CK8 positive and AR positive (prostate epithelial markers). Scale bars, 50 μm. Mets, metastasis; LN, lymph node.

FIG. 5 demonstrates that the 284 PCDETERMINANTS from Table 1A predict human prostate cancer aggressiveness and metastasis. In this particular experiment, the 284 PCDETERMINTS listed on Table 1A were derived from a comparison of 3 tumor samples from Pten and 3 tumor samples form Pten Smad4. The 284 PCDETERMINANTS from Table 1A were evaluated for prognostic utility from the Glinsky et al (2004) prostate cancer gene expression data set. Biochemical recurrence (BCR) was defined by PSA levels (>0.2 ng/ml). Patient samples were categorized into two major clusters (High-risk and Low-Risk group) defined by the 284 PCDETERMINANTS listed on Table 1A.

FIG. 6 illustrates that Cell Movement genes are differentially expressed in the metastastic Smad4/Pten prostate tumors compares to indolent Pten tumors. Ingenuity Pathway Analysis (IPA) analysis on molecular functions of the differential expressed genes revealed that the cell movement genes ranks #18 vs. #1 for the Smad4/Pten prostate tumors when either are compared to Pten tumors. (A) IPA on molecular functions of differentially expressed genes between Pten^{pc-/-}; Smad4^{pc-/-} double mutants and Pten^{pc-/-} mice reveals that those genes have roles in cell movement, Cell Death, Cellular Growth and Proliferation, Cell-To-Cell Signaling and Interaction, Cellular Development, Cell Morphology, Cell Cycle, Cell Signaling, Post-Translational Modification, Lipid Metabolism, Small Molecule Biochemistry, Drug Metabolism, Vitamin and Mineral Metabolism, Cellular Function and Maintenance, Molecular Transport, Gene Expression, DNA Replication and Repair. Cell movement genes ranks #1. (B) IPA analysis on molecular functions of the differential expressed genes expressed between Pten^{pc-/-}; p53^{pc-/-} double mutants and Pten^{pc-/-} mice reveals that those genes have roles in Cell Death, Gene Expression, Cellular Growth and Proliferation, Cellular Development, Amino Acid Metabolism, Post-Translational Modification, Small Molecule Biochemistry, Cellular Function and Maintenance, Cell Morphology, Cellular Assembly and Organization, Cell Cycle, Cell-To-Cell Signaling and Interaction, Drug Metabolism, Lipid Metabolism, Molecular Transport, Cellular Compromise, Antigen Presentation, Cellular Movement, Carbohydrate Metabolism, RNA Damage and Repair, DNA Replication, and Repair, Nucleic Acid Metabolism, Cell Signaling, Protein Synthesis. In contrast to the Pten^{pc-/-}; Smad4^{pc-/-} tumors, the IPA of Pten^{pc-/-}; p53^{pc-/-} tumors show that cell movement genes ranks #18.

FIG. 7 illustrates gene profiling and promoter analysis reveals a subset of 66 putative Smad4 target genes differentially expressed between Pten^{pc-/-}; Smad^{pc-/-} double mutants and Pten^{pc-/-} mice. (A) 66 genes differentially expressed between Pten^{pc-/-}; Smad^{pc-/-} double mutants and Pten^{pc-/-} mice. (B) Ingenuity Pathway Analysis (IPA) on molecular functions reveals that these 66 genes have roles in cell movement, cancer, cellular growth and proliferation, and ell death.

FIG. 8 illustrates a 17 Smad-target gene signature can predictor cancer aggressiveness and metastasis. (A) A diagram representation of the development of 17 Smad target gene signature. Computer analysis reveal that there are 66 putative Smad-target gene among 284 genes differentially expressed between Pten^{pc-/-}; Smad^{pc-/-} double mutants and Pten^{pc-/-} mice. A 17 gene signature was developed based on the overlap with a human metastatic PCA dataset (B) 17 genes differentially expressed between Pten^{pc-/-}; Smad^{pc-/-} double mutants and Pten^{pc-/-} mice. (C) The 17 putative Smad target genes were subsequently evaluated for prognostic utility on a prostate cancer gene expression data set. Hierarchical clustering of the tumor samples (columns) and genes (rows) is provided. Red indicates high relative levels of gene expression, while green represents low relative levels of gene expression. Horizontal bars above the heat maps indicate the recurrence status of each patient (1, biochemical or tumor recurrence; 0, recurrence-free). Patients were categorized into two major clusters defined by the 17-gene signature. Lymph node and other distal metastasis are indicated by arrow in red. (D) Kaplan-Meier survival analysis based on the groups defined by the 17-gene cluster. (E, F) Same as C, 17-gene signature was evaluated in a breast adenocarcinoma dataset. Kaplan-Meier analysis was conducted for survival probability (E) and metastasis-free survival (F) based on the groups defined by the 17-gene cluster.

FIG. 9 illustrates that loss of Smad4 does not initiate prostate tumors up to 2 years age. Histopathological analysis (haematoxylin/eosin staining) of anterior prostates (AP) in Smad4 single mutants at one year (A) and two year of age (B) reveals normal glands in Smad^{pc-/-} mice. Scale bars, 50 μm.

FIG. 10 shows histopathological analysis of representative hydronephrosis in Pten^{pc-/-}; Smad^{pc-/-} mice. (A) Gross anatomy of representative Pten^{pc-/-}; Smad^{pc-/-} with prostate tumor at 26 weeks of age with a huge prostate tumor (dashed circle). Scale bars, 2 cm. (B, C) Histopathological analysis of representative kidney from Pten^{pc-/-} mice (B) and Pten^{pc-/-}; Smad^{pc-/-} mice with hydronephrosis (arrow) (C). Stained with hematoxylin and eosin (H&E). Scale bars, 1 mm.

FIG. 11 shows Microarray analysis of a subset of 284 (See Table 1A) cancer biology related genes differentially expressed between Pten^{pc-/-}; Smad^{pc-/-} double mutants and Pten^{pc-/-} mice. (A) 284 genes differentially expressed between Pten^{pc-/-}; Smad^{pc-/-} double mutants and Pten^{pc-/-} mice. (B) Ingenuity Pathway Analysis (IPA) on molecular functions reveals that these 284 genes have roles in cellular movement, cancer, cellular growth and proliferation, and cell death.

FIG. 12 (A) The 66 putative Smad target genes were subsequently evaluated for prognostic utility on a prostate cancer gene expression data set. Hierarchical clustering of the tumor samples (columns) and genes (rows) is provided. Red indicates high relative levels of gene expression, while green represents low relative levels of gene expression. Horizontal bars above the heat maps indicate the recurrence status of each patient (1, biochemical or tumor recurrence; 0, recurrence-free). Patients were categorized into two major clusters defined by the 66-gene signature. Lymph node and other distal metastasis are indicated by arrow in red. (B) Kaplan-Meier survival analysis based on the groups defined by the 66-gene cluster.

FIG. 13 shows that Smad4 loss can circumvent cellular senescence elicited by Pten loss in primary mouse embryonic fibroblasts (MEFs) through p53-dependent pathway. (A) senescence staining of WT (Panel a), Smad^{-/-} (Panel b), Pten^{-/-} (Panel c), and Pten^{-/-}; Smad^{-/-} (Panel d) MEFs. Representative sections from three independent MEFs of each genotype. (B) Quantification of the β-Gal staining. Error bars represent s.d. for a representative experiment performed in triplicate. Asterisk indicates statistical significance between Pten^{pc-/-} and Pten^{pc-/-}; Smad^{pc-/-} double mutants (P<0.05). (C) Western blot analysis of MEFs from each genotype shows p53 expression level for a representative experiment performed in duplicate (of more than four mice per genotype). Actin was used as an internal loading control.

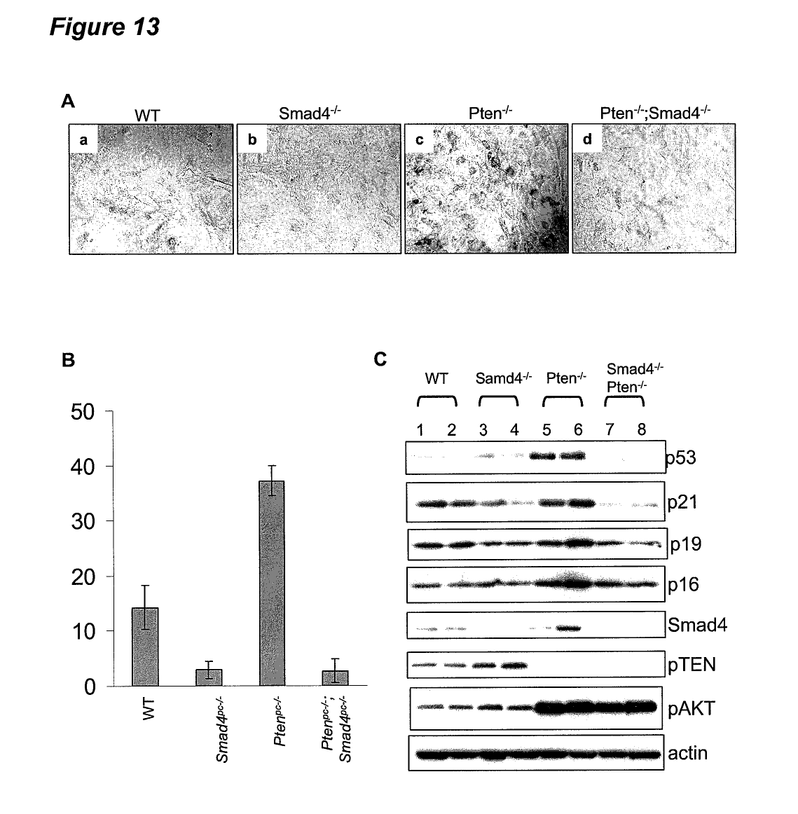

FIG. 14 shows prostate epithelial cells from Pten^{pc-/-}; Smad^{pc-/-} double mutants form orthotopic metastatic tumors with prostate epithelial cell markers in nude mice. (A) Orthotopic injection of prostate epithelial cells from Pten^{pc-/-}; Smad^{pc-/-} double mutants form tumor in prostate (dashed circle) and form lung metastasis (arrows). Scale bars, 1 cm. (B) Immunohistochemical analyses show that orthotopic tumors and lung metastasis are CK8 positive and #AR positive (prostate epithelial markers). Scale bars, 50 μm.

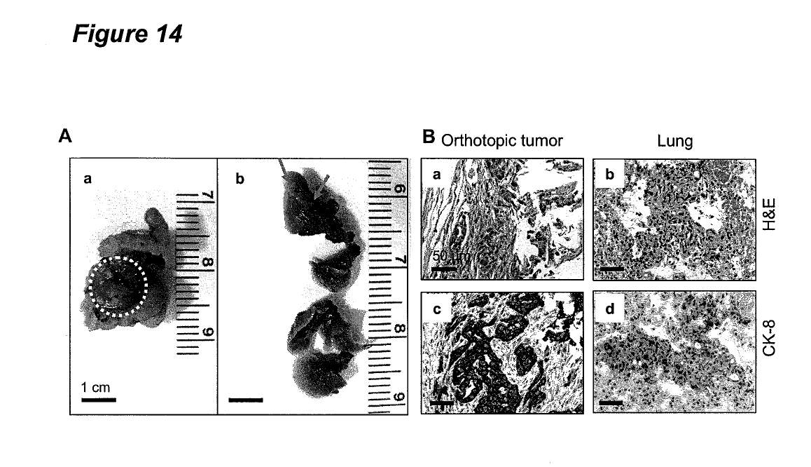

FIG. 15 shows Prostate epithelial cells from Pten^{pc-/-}; Smad^{pc-/-} double mutants form orthotopic metastatic tumors with prostate epithelial cell markers in nude mice. (A) Kidney implantation of prostate epithelial cells from Pten^{pc-/-}; Smad^{pc-/-} double mutants form tumor in prostate (dashed circle) and form lung metastasis (arrows). Scale bars, 1 cm. (B) Immunohistochemical analyses show that kidney tumors and lung metastasis are CK8 positive and #AR positive (prostate epithelial markers). Scale bars, 50 μm

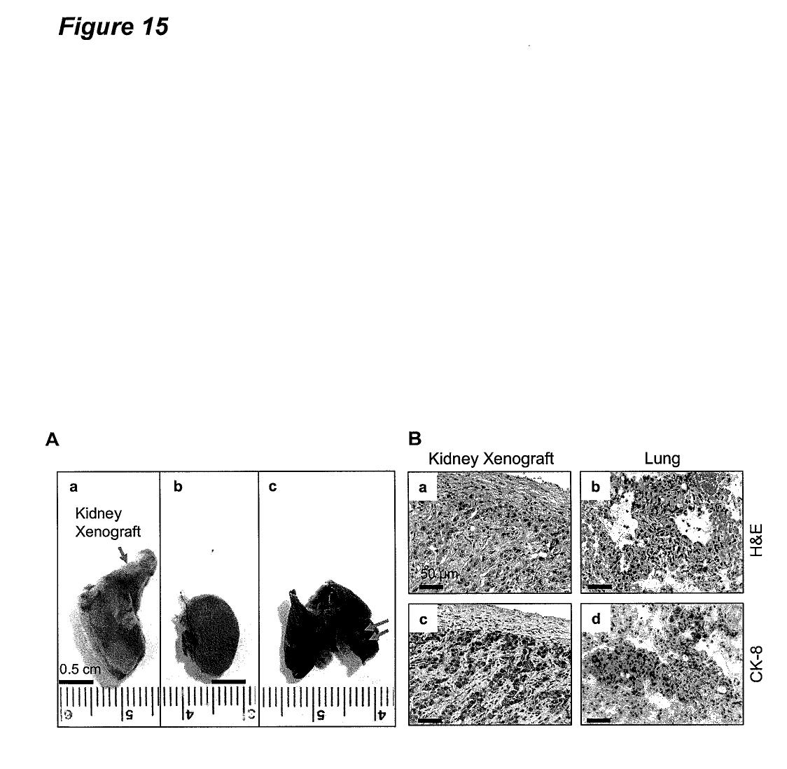

FIG. 16 shows that restoration of Smad4 in Pten-Smad4 double null prostate tumor cells decreases cell viability when treated with TGFβ1. (A) The restoration of Smad4 in Smad4-deficient prostate cancer cells decreases cell viability upon treatment with TGFβ1. Parental control cells (Contl) and Smad4-Tet on cells (Smad4) were treated with 0.016 ng/mL, 0.031 ng/mL, 0.063 ng/mL, 0.125 ng/mL, 0.25 ng/mL, 0.5 ng/mL TGFβ1 in the presence or absence of 1 μg/mL doxycycline (Dox) in 5% charcoal-stripped FBS-containing medium, and then cell viability was assayed by adenosine triphosphate quantitation. Error bars represent s.d. for a representative experiment performed in triplicate. Black bars, control parental line without Dox; blue bars, control parental line with Dox; red bars, Smad4 tet-on line without Dox; green bars, Smad4 tet on line with Dox. (B) Western blot analysis of Smad4 expression upon Dox treatment shows Smad4 expression in Smad4 tet-on line with treatment of Dox or without the treatment of Dox. Ran was used as an internal loading control. (C) Morphology of cells with or without TGFβ1 treatment. The cells were photographed after 4 d of treatment with TGFβ1 or vehicle.

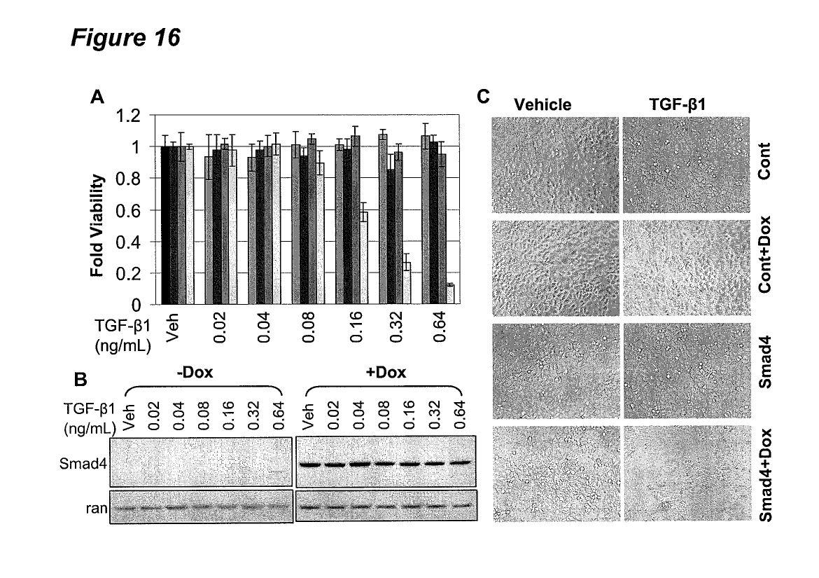

FIG. 17 shows loss of Smad4 circumvented Pten-loss-induced autophagy. (A) Morphology of cells with or without TGFβ1 treatment. The cells were photographed after 3 days of treatment with TGFβ1 or vehicle. (B) Transmission electron microscopy of prostate tumor cells from Pten^{pc-/-}; Smad^{pc-/-} double mutants and Pten^{pc-/-} mouse at 15 weeks of age.

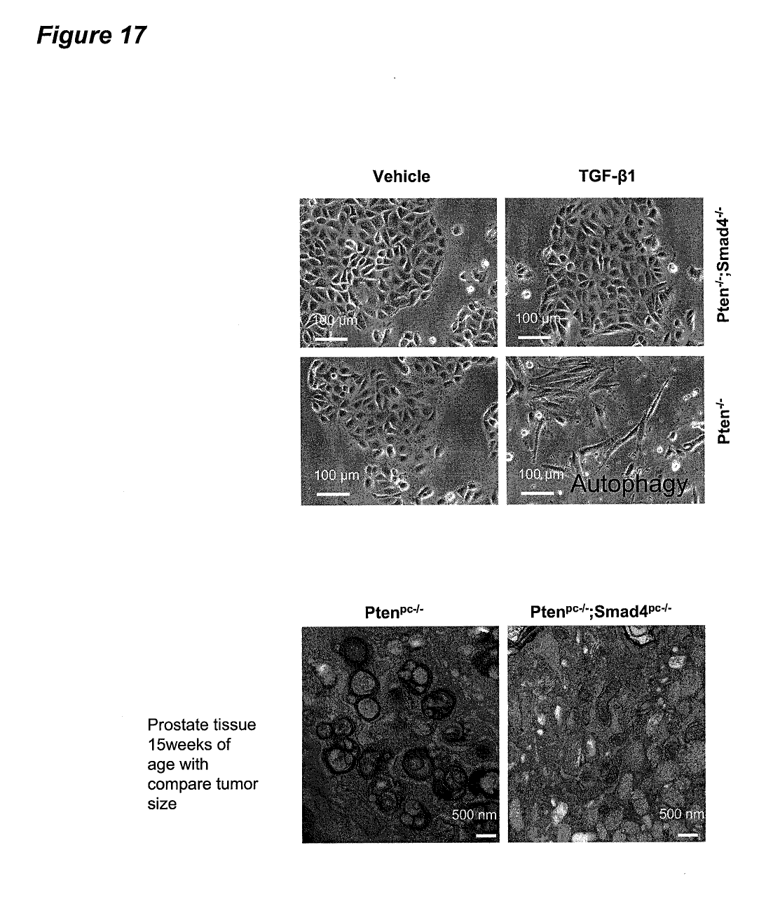

FIG. 18 demonstrates that Pten/Smad4 double mutant mice with hormone ablation via castration developed hormone-refractory metastatic PCA. (A) Kaplan-Meier overall cumulative survival analysis of castrated animals. A statistically significant extension in lifespan (P<0.0001) compared with the castration-free Pten^{pc-/-}; Smad^{pc-/-} cohort (n=20) was found for the castrated Pten^{pc-/-}; Smad^{pc-/-} cohort (n=22) (asterisk). The arrow indicates the castration at 15 weeks of age. (B) Castration did not block metastasis of prostate cancer in Pten^{pc-/-}; Smad^{pc-/-} double mutants. A higher magnified picture (boxed region) is shown on the right (panel b). Histopathological analysis of representative lymph node metastasis. Scale bars, 200 μm for panel a and 50 μm for panel b. (C) Histopathological and proliferation analysis revealed high proliferation (brown staining) in castrated Pten^{pc-/-}; Smad^{pc-/-} double mutants, compared with castrated WT and Pten^{pc-/-} mice. H&E, haematoxylin/eosin. Scale bars, 50 μm. Analysis was performed on 23-week-old mice which were castrated at 15-week-old. (D) Quantification of brdu pulse labeling of 23-week-old mice which were castrated at 15-week-old. Representative sections from three mice were counted for each genotype. Asterisk indicates statistical significance between Pten^{pc-/-}; Smad^{pc-/-} double mutants and Pten^{pc-/-} (P<0.05).

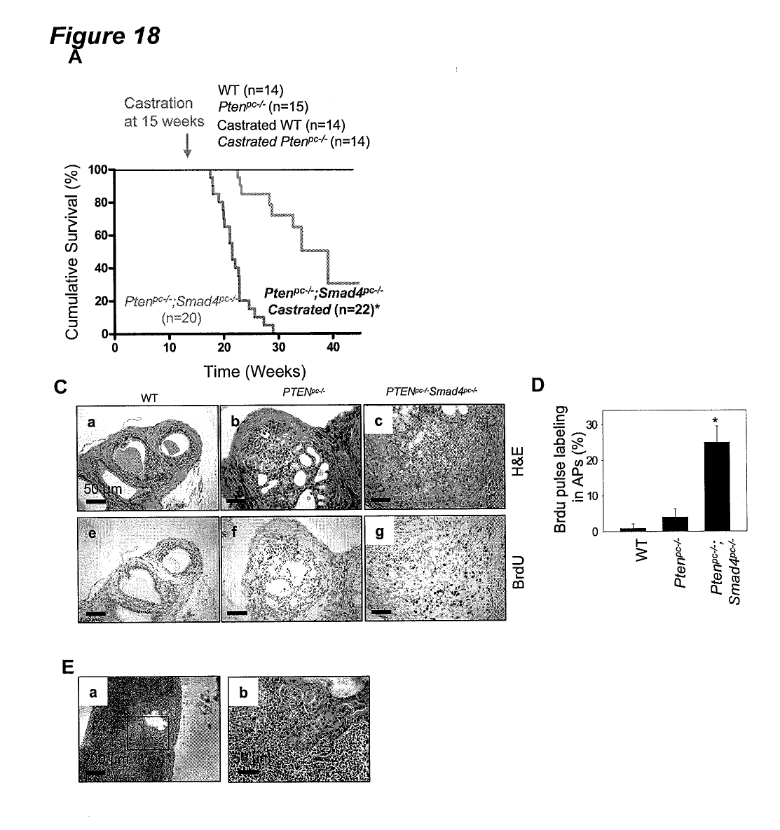

FIG. 19 illustrates the model of how Pten and Smad4 cooperate to control prostate cancer initiation and progression. Pten loss in prostate result in the development of prostate tumor, but further progression was suppressed by proliferative block/senescence induced by Pten loss. Both Pten and Smad4 loss circumvent the Pten-loss-induced proliferative block/senescence and possibly other cellular and intracellular suppression mechanisms such as those impeding cellular movement through PCDETERMINANTS 1-372 or a subset of PCDETERMINANTS 1-372, and eventually led to the prostate tumor cells to progress to metastasis.

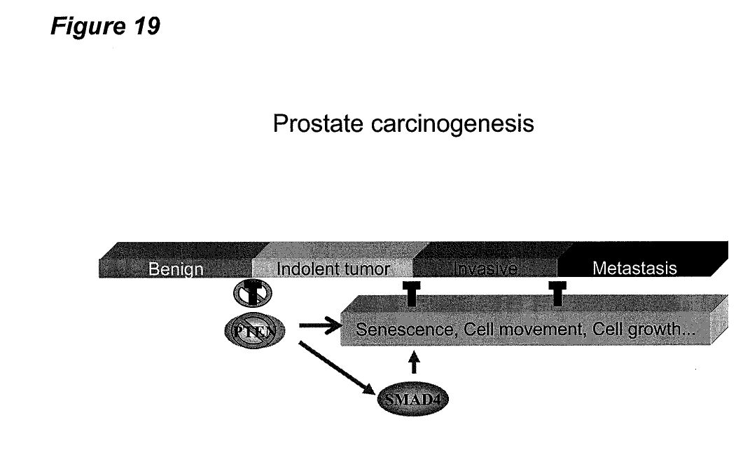

FIG. 20 demonstrates cross-species triangulated differentially expressed genes between Pten^{pc-/-}; Smad4^{pc-/-} double mutants and Pten^{pc-/-} mice are linked to clinical outcome in human PCA. (A) A diagram representation of the development of a 56 gene set based on the overlap of differentially expressed genes between Pten^{pc-/-}; Smad4^{pc-/-} double mutants and Pten^{pc-/-} mice (Table 1B) with a human metastatic PCA dataset¹⁹. (B) The 56 gene set (TABLE 7) was subsequently evaluated for prognostic utility on a prostate cancer gene expression data set. Patient samples were categorized into two major clusters (low risk group and high risk group) defined by the 56-gene signature. Kaplan-Meier analysis of biochemical recurrence (BCR) PSA level (>0.2 ng/ml) based on the groups defined by the 56-gene cluster. A statistically significant for BCR PSA recurrence-free survival (P=0.0018) compared with the “low-risk” cohort was found for the “high-risk” cohort.

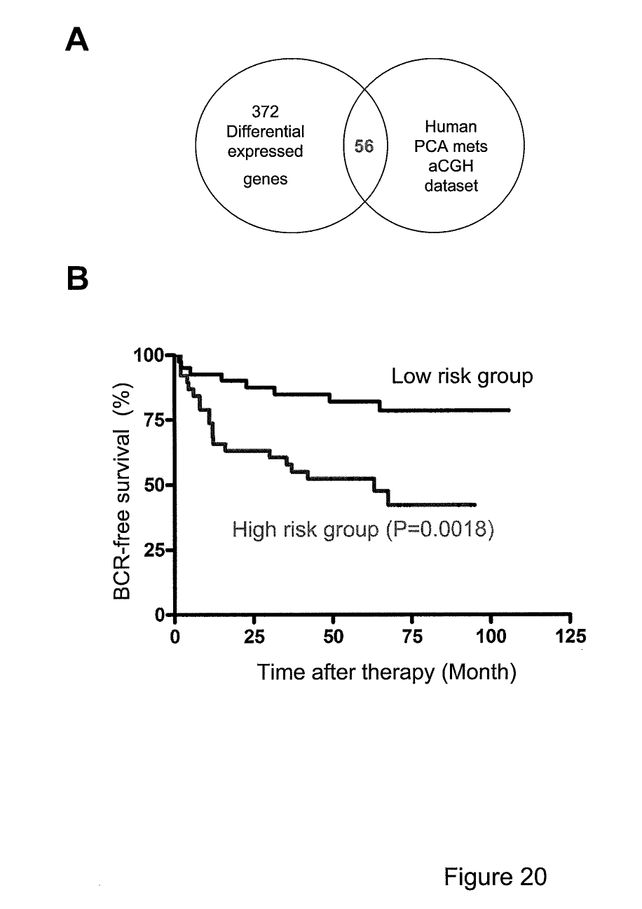

FIG. 21 illustrates approaches to identify PCDETERMINANTS that functionally drive or inhibit invasion in vitro.

FIG. 22 demonstrates use of the invasion assay to functionally validate candidate genes. A representative Boyden chamber invasion assay with PC3 cells overexpressing SPP1 and or GFP control in triplicates. (A) Enforced expression of SPP1 confirmed its capability to significantly enhance invasive activity of human PCA PC3 cells by invasion assay. (B) Bar graph indicates statistical significance between enforced SPP1 and GFP control (P<0.05). (C) The table confirms the assay identifies invasion-promoting genes that are annotated as being involved in cellular movement, but also genes not classified as being involved in movement yet drive invasive and metastatic properties in vitro. A significantly higher frequency (P=0.02, Fisher's Exact Test) of invasion-validated PCDETERMINANTS are annotated as cellular movement genes compared to those not from the cellular movement annotated genes.

FIG. 23 demonstrates a FOUR (4) PCDETERMINANT gene signature PTEN-SMAD4-Cyclin D1-SPP1 which was informed by the Pten/Smad4 transcriptome data, the histopathological data and invasion validation data is linked to clinical outcome in human PCA. (A) Dysregulated Pten and Smad4 expression together with the related Cyclin D1 (proliferation/senescence) and SPP1 (motility network) was subsequently shown to be correlated with the human prostate cancer progression on a prostate cancer gene expression data set. Patient samples were categorized into two major clusters by K-mean (High-risk and Low risk groups) defined by the PTEN, SMAD4, Cyclin D1, and SPP1 signature. High-risk group patient showed statistically significant in biochemical recurrence (BCR) PSA level (>0.2 ng/ml) by Kaplan-Meier analysis. (B) The significant correlation of PTEN, SMAD4, Cyclin D1, and SPP1 signature in PCA progression was validated in an independent Physicians' Health Study (PHS) dataset with c-statistic. The PTEN, SMAD4, Cyclin D1, and SPP1 show similar power to Gleason score in the prediction of lethal outcomes. The addition of PTEN, SMAD4, Cyclin D1, and SPP1 genes to Gleason significantly improves prediction of lethal outcomes over the model of Gleason alone in PHS. Moreover, PTEN, SMAD4, Cyclin D1, and SPP1 4-gene set ranked as the most enriched among 244 bidirectional signatures curated in the Molecular Signature Databases of the Broad Institute (MSigDB, version 2.5), indicating the robust significance of this 4 gene signature in prediction of lethal outcome.

FIG. 24 demonstrates cross-species triangulated differentially expressed genes between Pten^{pc-/-}; Smad4^{pc-/-} double mutants and Pten^{pc-/-} mice are linked to clinical outcome in human breast cancer. (A) The 56 gene set (TABLE 7) was subsequently evaluated for prognostic utility on a breast adenocarcinoma dataset. Patient samples were categorized into two major clusters (low risk group and high risk group) defined by the 56-gene signature. Kaplan-Meier analysis was conducted for survival probability (p=0.00358) (A) and metastasis-free survival (p=00492) (B) based on the groups defined by the 56-gene cluster.

FIG. 25 demonstrates that both prostate and breast cancer progression correlated PCDETERMINANTS are highly linked to clinical outcome in human breast cancer. (A) The 20 PCDETERMINANTS exhibiting progression correlated expression in both prostate cancer and breast cancer (Table 6) was evaluated for prognostic utility on a breast adenocarcinoma dataset. Patient samples were categorized into two major clusters (low risk group and high risk group) defined by the 20 progression correlated-gene signature. Kaplan-Meier analysis was conducted for survival probability (p=2.93e⁻¹¹) (A) and metastasis-free survival (p=4.62e⁻¹⁰) (B) based on the groups defined by the 20 PCDETERMINANTS.

### DETAILED DESCRIPTION OF THE INVENTION

The present invention relates to the identification of signatures associated with and PCDETERMINANTS conferring subjects with metastatic prostate cancer or are at risk for developing metastatic prostate cancer. The invention further provides a murine mouse model for invasive and metastatic prostate cancer, where the mouse prostate epithelium sustains deletion, or other means of mutational or epigenetic extinction of an initiating lesion such as the Pten and Smad4 gene. It would be recognized by one skilled in the art that other initiating lesion, including over-expression of oncogene trangenes could be coupled to the Smad4 deletion to enable malignant progression. This mouse model can be used to identify cancer detection biomarkers.

Human cancers harbor innumerable genetic and epigenetic alterations presenting formidable challenges in deciphering those changes that drive the malignant process and dictate a given tumor's clinical behavior. The need for accurately predictive biomarkers reflective of a tumor's malignant potential is evident across many cancer types, particularly prostate cancer, where current management algorithms result in either under-treatment with consequent risk of death or exposure to unnecessary morbid treatments.

Genetically engineered mouse models have been shown to be tremendously powerful as “filters” to mine highly complex genomic datasets in human. In particular, these refined genetically engineered mouse models of human cancers have been documented in high-resolution comparative oncogenomic analyses to harbor substantial overlap in cancer-associated transcriptional and chromosomal DNA aberrations patterns—the latter resulting in the rapid and efficient identification of many novel cancer genes. Similar cross-species comparisons of the serum proteome have also proven effective in the identification of early detection biomarkers for pancreas cancer in humans. Thus, it stands to reasons that development of a valid mouse model recapitulating the disease state of metastasis driven by bona fide human prostate cancer genes will greatly facilitate our efforts to develop prognostic and early detection biomarkers and possible therapeutic targets.

Global transcriptome analyses of indolent Pten deficient prostate PIN lesions inferred the presence of a Smad4-dependent checkpoint which induces a senescence response in setting of Pten inactivation, blocking progression beyond PIN. Concomitant Smad4 deletion in the mouse prostate epithelium along with Pten deletion indeed generated a fulminant metastatic prostate model with short latency, providing unequivocal genetic proof of this hypothesis. That this is a mouse model of metastatic prostate cancers driven by bona fide prostate tumor suppressors is supported by the demonstration of consistent Smad4 downregulation during progression from primary to metastatic PCA in human. The validity of this model was further re-enforced by demonstration that the 17 predicted direct targets of Smad4 conserved across two species are capable of stratifying human prostate and breast adenocarcinomas into two groups with significant differences in outcome as measured by recurrence or survivals. Therefore, the inventors have established a bona fide genetically engineered mouse model of metastatic PCA, enabling future mechanistic studies as well as comparative genomic and proteomic analyses in searches for prognostic and early-detection biomarkers.

It has been established that loss of Pten function is one of the most significant genetic events in prostate carcinogenesis. Loss of Pten results in prostate tumorigenesis in the mouse prostate; however, it also provokes cellular senescence which may function as a further level of tumor suppressor to block the tumor cells progression to an invasive stage. Overriding senescence induced by Pten through inactivation of p53 contributes to the progression of prostate tumors from an indolent lesion to an invasive tumor. The inventors have discovered that Smad4 loss also can circumvent cellular senescence elicited by Pten loss. Overriding senescence by loss of Smad4 is cooperative to Pten loss and may contribute its role in the promotion of tumor cells. This is also in agreement with the previous report that circumvention of cellular senescence by p53 loss is cooperative to Pten loss and contributes to the prostate tumor progression to a modestly invasive but non-metastatic lesion. This unique Pten/Smad4 model system therefore provides a tool to further dissect the molecular events for this important tumor biological process in the future.

Although circumvention of senescence results in Pten/Smad4 double mutant mouse prostate tumor cell progression to an invasive and metastatic state, circumvention of senescence in mouse model with Pten/p53 inactivation does not result in metastasis. Inactivation of Pten alone in mouse prostate can generate some feeble metastasis phenotype at very old age (more than one year) in a small portion of Pten mice (2 in 8). These observations indicated that additional genetic or epigenetic alterations besides Pten loss are needed for the prostate tumor cells to achieve a metastatic state. Circumvention of cellular senescence may be a pre-requisite for progression but other biological processes are likely needed such as deactivation of autophagy to achieve a robust metastatic state. In support of the presence of other biological processes, we observed that reconstitution of Smad4 in the Pten/Smad deficient tumor cells does not reinstate senescence yet renders cells non-metastatic. Specifically, we established an inducible Smad4 tet-on system to restore Smad4 expression in a time-dependent and dose dependent manner. It was found that restoration of Smad4 can sensitize the tumor to cell death in response to the treatment of TGFβ.

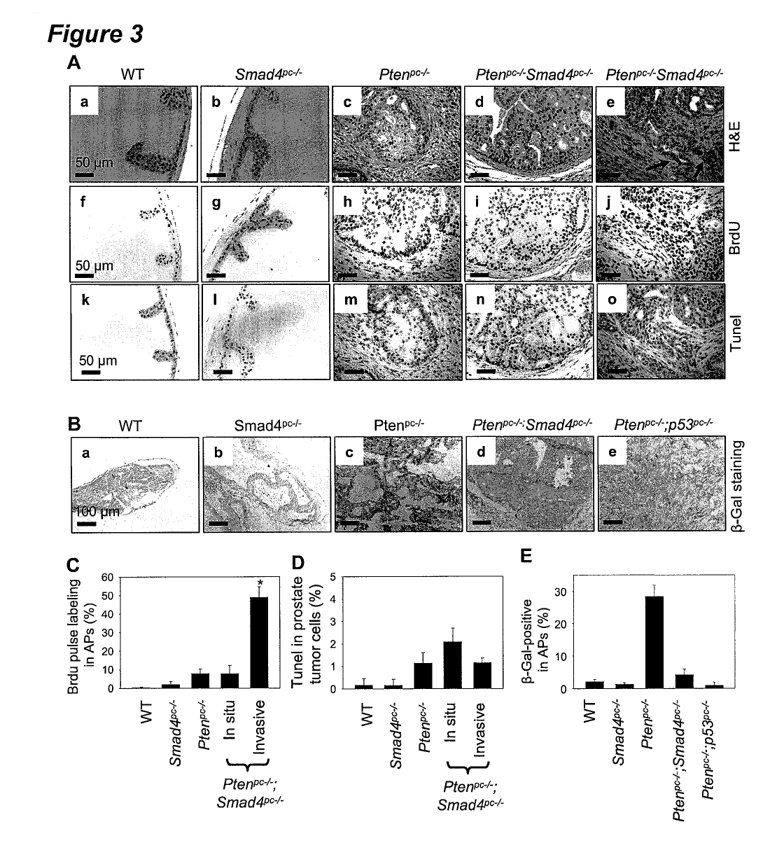

The canonical TGFβ-Smad pathway starts from the ligand-receptor complex and ends in the nucleus. Upon TGFβ superfamily ligand binding, receptor-phosphorylated R-Smads oligomerizes with Smad4 and translocate to the nucleus and bind directly to Smad-binding elements on DNA where they can induce or repress a diverse array of genes. In benign prostatic epithelia, by eliciting differentiation, inhibiting proliferation, and inducing apoptosis, TGF-β provides a mechanism to maintain homeostasis in the prostate. Thus, it was speculated that this major arm of the TGFβ plays a critical role in the prostate tumor progression suppression. The tumor suppressor role of TGFβ signaling is underscored by the presence of inactivating TGFβ receptor mutations and the extinction of Smad2, Smad3, and Smad4 proteins in multiple cancers including prostate cancer. Although TGFβ was shown to inhibit many normal cell types and tumor cell growth, TGFβ was also reported to enhance malignant potential of epithelial tumors, including proliferation, migration, and epithelial-to-mesenchymal transition (EMT)-a process by which advanced carcinomas acquire a highly invasive, undifferentiated and metastatic phenotype. Most recently, it has been demonstrated that TGFβ in the breast tumor microenvironment can prime cancer cells for metastasis to the lungs though induction of angiopoietin-like 4 (ANGPTL4) by TGFβ via the Smad signaling pathway. These paradoxical activities of tumor suppression and promotion are probably dependent on the activities of other signaling pathways in given cells, which are dictated by the different cell contexts as well as the interplay with other tissue. The Pten/Smad4 model has now clarified the role of the TGFb pathway in prostate cancer by clearly showing that Smad4 loss is not sufficient alone to initiate the development of prostate lesion, but promotes acceleration and progression of prostate tumor to metastasis with complete penetrance, at least on the background of Pten deficiency (FIG. 3). The Pten/Smad4 model study clearly demonstrated that Smad4 loss can override the senescence induced by Pten loss. Since override senescence by p53 loss in Pten deficiency background result in progression of indolent prostate tumor to invasive lesion, but not to metastasis. Senescence is thus considered to be an early barrier during the prostate tumorigenesis from indolent to invasive status. As restoration of Smad4 back into the Pten/Smad4 double mutant prostate tumor cells did not restore the senescence (data not shown). However, restoration of Smad4 decreased the viability of the cells upon the treatment of TGFβ1. The senescence barrier may be, therefore, a transient cellular response to the oncogenic signal(s) to block tumor progression.

Additionally, molecularly comparative transcriptomic analyses of equivalent early stage Pten and Pten/Smad null prostate tumors (n=5 for each genotype) revealed differential expression of 372 genes of which at least 66 genes contain Smad binding elements in their promoters. Through cross-species integration with copy number profiles of human metastatic prostate tumors, we identified 17 of these Smad4 targets that are strongly associated with risk of recurrence in human prostate cancer and with metastasis risk and survival in breast cancer, thereby supporting the human relevance of this novel metastatic prostate model and its use in the discovery of genetic PCDETERMINANTS governing disease progression across many tumor types through comparative oncogenomics.

Accordingly, the invention provides an animal model for metastatic prostate cancer. The animal model of the instant invention thus finds particular utility as a screening tool to elucidate the mechanisms of the various genes involved in both normal and diseased patient populations.

The invention also provides methods for identifying subjects who have metastatic prostate cancer, or who at risk for experiencing metastatic prostate cancer by the detection of PCDETERMINANTS associated with the metastatic tumor, including those subjects who are asymptomatic for the metastatic tumor. These signatures and PCDETERMINANTS are also useful for monitoring subjects undergoing treatments and therapies for cancer, and for selecting or modifying therapies and treatments that would be efficacious in subjects having cancer, wherein selection and use of such treatments and therapies slow the progression of the tumor, or substantially delay or prevent its onset, or reduce or prevent the incidence of tumor metastasis.

### DEFINITIONS

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“PCDETERMINANTS in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. PCDETERMINANTS can also include mutated proteins or mutated nucleic acids. PCDETERMINANTS also encompass non-blood borne factors or non-analyte physiological markers of health status, such as “clinical parameters” defined herein, as well as “traditional laboratory risk factors”, also defined herein. PCDETERMINANTS also include any calculated indices created mathematically or combinations of any one or more of the foregoing measurements, including temporal trends and differences. Where available, and unless otherwise described herein, PCDETERMINANTS which are gene products are identified based on the official letter abbreviation or gene symbol assigned by the international Human Genome Organization Naming Committee (HGNC) and listed at the date of this filing at the US National Center for Biotechnology Information (NCBI) web site (http://www.ncbi.nlm.nih.gov/sites/entrez?db=gene), also known as Entrez Gene.

“PCDETERMINANT” OR “PCDETERMINANTS” encompass one or more of all nucleic acids or polypeptides whose levels are changed in subjects who have metastatic prostate cancer or are predisposed to developing metastatic prostate cancer, or at risk of metastatic prostate cancer. Individual PCDETERMINANTS are summarized in Table 1B and are collectively referred to herein as, inter alia, “metastatic tumor-associated proteins”, “PCDETERMINANT polypeptides”, or “PCDETERMINANT proteins”. The corresponding nucleic acids encoding the polypeptides are referred to as “metastatic tumor-associated nucleic acids”, “metastatic tumor-associated genes”, “PCDETERMINANT nucleic acids”, or “PCDETERMINANT genes”. Unless indicated otherwise, “PCDETERMINANT”, “metastatic tumor-associated proteins”, “metastatic tumor-associated nucleic acids” are meant to refer to any of the sequences disclosed herein. The corresponding metabolites of the PCDETERMINANT proteins or nucleic acids can also be measured, as well as any of the aforementioned traditional risk marker metabolites.

Physiological markers of health status (e.g., such as age, family history, and other measurements commonly used as traditional risk factors) are referred to as “PCDETERMINANT physiology”. Calculated indices created from mathematically combining measurements of one or more, preferably two or more of the aforementioned classes of PCDETERMINANTS are referred to as “PCDETERMINANT indices”.

“Clinical parameters” encompasses all non-sample or non-analyte biomarkers of subject health status or other characteristics, such as, without limitation, age (Age), ethnicity (RACE), gender (Sex), or family history (FamHX).

“Circulating endothelial cell” (“CEC”) is an endothelial cell from the inner wall of blood vessels which sheds into the bloodstream under certain circumstances, including inflammation, and contributes to the formation of new vasculature associated with cancer pathogenesis. CECs may be useful as a marker of tumor progression and/or response to antiangiogenic therapy.

“Circulating tumor cell” (“CTC”) is a tumor cell of epithelial origin which is shed from the primary tumor upon metastasis, and enters the circulation. The number of circulating tumor cells in peripheral blood is associated with prognosis in patients with metastatic cancer. These cells can be separated and quantified using immunologic methods that detect epithelial cells, and their expression of PCDETERMINANTS can be quantified by qRT-PCR, immunofluorescence, or other approaches.

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining PCDETERMINANTS and other PCDETERMINANTS are linear and non-linear equations and statistical classification analyses to determine the relationship between levels of PCDETERMINANTS detected in a subject sample and the subject's risk of metastatic disease. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a PCDETERMINANT selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

“Negative predictive value” or “NPV” is calculated by TN/(TN+FN) or the true negative fraction of all negative test results. It also is inherently impacted by the prevalence of the disease and pre-test probability of the population intended to be tested.

See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronary Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

Finally, hazard ratios and absolute and relative risk ratios within subject cohorts defined by a test are a further measurement of clinical accuracy and utility. Multiple methods are frequently used to defining abnormal or disease values, including reference limits, discrimination limits, and risk thresholds.

“Analytical accuracy” refers to the reproducibility and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Positive predictive value” or “PPV” is calculated by TP/(TP+FP) or the true positive fraction of all positive test results. It is inherently impacted by the prevalence of the disease and pre-test probability of the population intended to be tested.

“Risk” in the context of the present invention, relates to the probability that an event will occur over a specific time period, as in the conversion to metastatic events, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a primary tumor to metastatic prostate cancer or to one at risk of developing a metastatic, or from at risk of a primary metastatic event to a more secondary metastatic event. Risk evaluation can also comprise prediction of future clinical parameters, traditional laboratory risk factor values, or other indices of cancer, either in absolute or relative terms in reference to a previously measured population. The methods of the present invention may be used to make continuous or categorical measurements of the risk of metastatic prostate cancer thus diagnosing and defining the risk spectrum of a category of subjects defined as being at risk for metastatic tumor. In the categorical scenario, the invention can be used to discriminate between normal and other subject cohorts at higher risk for metastatic tumors. Such differing use may require different PCDETERMINANT combinations and individualized panels, mathematical algorithms, and/or cut-off points, but be subject to the same aforementioned measurements of accuracy and performance for the respective intended use.

A “sample” in the context of the present invention is a biological sample isolated from a subject and can include, by way of example and not limitation, tissue biopsies, whole blood, serum, plasma, blood cells, endothelial cells, circulating tumor cells, lymphatic fluid, ascites fluid, interstitial fluid (also known as “extracellular fluid” and encompasses the fluid found in spaces between cells, including, inter alia, gingival cevicular fluid), bone marrow, cerebrospinal fluid (CSF), saliva, mucous, sputum, sweat, urine, or any other secretion, excretion, or other bodily fluids.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of tumor metastasis. A subject can be male or female. A subject can be one who has been previously diagnosed or identified as having primary tumor or a metastatic tumor, and optionally has already undergone, or is undergoing, a therapeutic intervention for the tumor. Alternatively, a subject can also be one who has not been previously diagnosed as having metastatic prostate cancer. For example, a subject can be one who exhibits one or more risk factors for metastatic prostate cancer.

“TN” is true negative, which for a disease state test means classifying a non-disease or normal subject correctly.

“TP” is true positive, which for a disease state test means correctly classifying a disease subject.

“Traditional laboratory risk factors” correspond to biomarkers isolated or derived from subject samples and which are currently evaluated in the clinical laboratory and used in traditional global risk assessment algorithms. Traditional laboratory risk factors for tumor metastasis include for example Gleason score, depth of invasion, vessel density, proliferative index, etc. Other traditional laboratory risk factors for tumor metastasis are known to those skilled in the art.

Methods and Uses of the Invention

The methods disclosed herein are used with subjects at risk for developing metastatic prostate cancer, or other cancer subjects, such as those with breast cancer who may or may not have already been diagnosed with metastatic prostate cancer or other cancer types and subjects undergoing treatment and/or therapies for a primary tumor or metastatic prostate cancer and other cancer types. The methods of the present invention can also be used to monitor or select a treatment regimen for a subject who has a primary tumor or metastatic prostate cancer and other cancer types, and to screen subjects who have not been previously diagnosed as having metastatic prostate cancer and other cancer types, such as subjects who exhibit risk factors for metastasis. Preferably, the methods of the present invention are used to identify and/or diagnose subjects who are asymptomatic for metastatic prostate cancer and other cancer types. “Asymptomatic” means not exhibiting the traditional signs and symptoms.

The methods of the present invention may also used to identify and/or diagnose subjects already at higher risk of developing metastatic prostate cancer and other metastatic cancer types based on solely on the traditional risk factors.

A subject having metastatic prostate cancer and other metastatic cancer types can be identified by measuring the amounts (including the presence or absence) of an effective number (which can be two or more) of PCDETERMINANTS in a subject-derived sample and the amounts are then compared to a reference value. Alterations in the amounts and patterns of expression of biomarkers, such as proteins, polypeptides, nucleic acids and polynucleotides, polymorphisms of proteins, polypeptides, nucleic acids, and polynucleotides, mutated proteins, polypeptides, nucleic acids, and polynucleotides, or alterations in the molecular quantities of metabolites or other analytes in the subject sample compared to the reference value are then identified.

A reference value can be relative to a number or value derived from population studies, including without limitation, such subjects having the same cancer, subject having the same or similar age range, subjects in the same or similar ethnic group, subjects having family histories of cancer, or relative to the starting sample of a subject undergoing treatment for a cancer. Such reference values can be derived from statistical analyses and/or risk prediction data of populations obtained from mathematical algorithms and computed indices of cancer metastasis. Reference PCDETERMINANT indices can also be constructed and used using algorithms and other methods of statistical and structural classification.

In one embodiment of the present invention, the reference value is the amount of PCDETERMINANTS in a control sample derived from one or more subjects who are not at risk or at low risk for developing metastatic tumor. In another embodiment of the present invention, the reference value is the amount of PCDETERMINANTS in a control sample derived from one or more subjects who are asymptomatic and/or lack traditional risk factors for metastatic prostate cancer. In a further embodiment, such subjects are monitored and/or periodically retested for a diagnostically relevant period of time (“longitudinal studies”) following such test to verify continued absence of metastatic prostate cancer (disease or event free survival). Such period of time may be one year, two years, two to five years, five years, five to ten years, ten years, or ten or more years from the initial testing date for determination of the reference value. Furthermore, retrospective measurement of PCDETERMINANTS in properly banked historical subject samples may be used in establishing these reference values, thus shortening the study time required.

A reference value can also comprise the amounts of PCDETERMINANTS derived from subjects who show an improvement in metastatic risk factors as a result of treatments and/or therapies for the cancer. A reference value can also comprise the amounts of PCDETERMINANTS derived from subjects who have confirmed disease by known invasive or non-invasive techniques, or are at high risk for developing metastatic tumor, or who have suffered from metastatic prostate cancer.

In another embodiment, the reference value is an index value or a baseline value. An index value or baseline value is a composite sample of an effective amount of PCDETERMINANTS from one or more subjects who do not have metastatic tumor, or subjects who are asymptomatic a metastatic. A baseline value can also comprise the amounts of PCDETERMINANTS in a sample derived from a subject who has shown an improvement in metastatic tumor risk factors as a result of cancer treatments or therapies. In this embodiment, to make comparisons to the subject-derived sample, the amounts of PCDETERMINANTS are similarly calculated and compared to the index value. Optionally, subjects identified as having metastatic tumor, or being at increased risk of developing metastatic prostate cancer are chosen to receive a therapeutic regimen to slow the progression the cancer, or decrease or prevent the risk of developing metastatic prostate cancer.

The progression of metastatic prostate cancer, or effectiveness of a cancer treatment regimen can be monitored by detecting a PCDETERMINANT in an effective amount (which may be two or more) of samples obtained from a subject over time and comparing the amount of PCDETERMINANTS detected. For example, a first sample can be obtained prior to the subject receiving treatment and one or more subsequent samples are taken after or during treatment of the subject. The cancer is considered to be progressive (or, alternatively, the treatment does not prevent progression) if the amount of PCDETERMINANT changes over time relative to the reference value, whereas the cancer is not progressive if the amount of PCDETERMINANTS remains constant over time (relative to the reference population, or “constant” as used herein). The term “constant” as used in the context of the present invention is construed to include changes over time with respect to the reference value.

For example, the methods of the invention can be used to discriminate the aggressiveness/and or accessing the stage of the tumor (e.g. Stage I, II, II or IV). This will allow patients to be stratified into high or low risk groups and treated accordingly.

Additionally, therapeutic or prophylactic agents suitable for administration to a particular subject can be identified by detecting a PCDETERMINANT in an effective amount (which may be two or more) in a sample obtained from a subject, exposing the subject-derived sample to a test compound that determines the amount (which may be two or more) of PCDETERMINANTS in the subject-derived sample. Accordingly, treatments or therapeutic regimens for use in subjects having a cancer, or subjects at risk for developing metastatic tumor can be selected based on the amounts of PCDETERMINANTS in samples obtained from the subjects and compared to a reference value. Two or more treatments or therapeutic regimens can be evaluated in parallel to determine which treatment or therapeutic regimen would be the most efficacious for use in a subject to delay onset, or slow progression of the cancer.

The present invention further provides a method for screening for changes in marker expression associated with metastatic prostate cancer, by determining the amount (which may be two or more) of PCDETERMINANTS in a subject-derived sample, comparing the amounts of the PCDETERMINANTS in a reference sample, and identifying alterations in amounts in the subject sample compared to the reference sample.

The present invention further provides a method of treating a patient with a tumor, by identifying a patient with a tumor where an effective amount of PCDETERMINANTS are altered in a clinically significant manner as measured in a sample from the tumor, an treating the patient with a therapeutic regimen that prevents or reduces tumor metastasis.

Additionally the invention provides a method of selecting a tumor patient in need of adjuvant treatment by assessing the risk of metastasis in the patient by measuring an effective amount of PCDETERMINANTS where a clinically significant alteration two or more PCDETERMINANTS in a tumor sample from the patient indicates that the patient is in need of adjuvant treatment.

Information regarding a treatment decision for a tumor patient by obtaining information on an effective amount of PCDETERMINANTS in a tumor sample from the patient, and selecting a treatment regimen that prevents or reduces tumor metastasis in the patient if two or more PCDETERMINANTS are altered in a clinically significant manner.

If the reference sample, e.g., a control sample, is from a subject that does not have a metastatic cancer, or if the reference sample reflects a value that is relative to a person that has a high likelihood of rapid progression to metastatic prostate cancer, a similarity in the amount of the PCDETERMINANT in the test sample and the reference sample indicates that the treatment is efficacious. However, a difference in the amount of the PCDETERMINANT in the test sample and the reference sample indicates a less favorable clinical outcome or prognosis.

By “efficacious”, it is meant that the treatment leads to a decrease in the amount or activity of a PCDETERMINANT protein, nucleic acid, polymorphism, metabolite, or other analyte. Assessment of the risk factors disclosed herein can be achieved using standard clinical protocols. Efficacy can be determined in association with any known method for diagnosing, identifying, or treating a metastatic disease.

The present invention also provides PCDETERMINANT panels including one or more PCDETERMINANTS that are indicative of a general physiological pathway associated with a metastatic lesion. For example, one or more PCDETERMINANTS that can be used to exclude or distinguish between different disease states or squeal associated with metastasis. A single PCDETERMINANT may have several of the aforementioned characteristics according to the present invention, and may alternatively be used in replacement of one or more other PCDETERMINANTS where appropriate for the given application of the invention.

The present invention also comprises a kit with a detection reagent that binds to two or more PCDETERMINANT proteins, nucleic acids, polymorphisms, metabolites, or other analytes. Also provided by the invention is an array of detection reagents, e.g., antibodies and/or oligonucleotides that can bind to two or more PCDETERMINANT proteins or nucleic acids, respectively. In one embodiment, the PCDETERMINANT are proteins and the array contains antibodies that bind two or more PCDETERMINANTS 1-372 sufficient to measure a statistically significant alteration in PCDETERMINANT expression compared to a reference value. In another embodiment, the PCDETERMINANTS are nucleic acids and the array contains oligonucleotides or aptamers that bind an effective amount of PCDETERMINANTS 1-372 sufficient to measure a statistically significant alteration in PCDETERMINANT expression compared to a reference value.

In another embodiment, the PCDETERMINANT are proteins and the array contains antibodies that bind an effective amount of PCDETERMINANTS listed on any one of Tables 1-7 sufficient to measure a statistically significant alteration in PCDETERMINANT expression compared to a reference value. In another embodiment, the PCDETERMINANTS are nucleic acids and the array contains oligonucleotides or aptamers that bind an effective amount of PCDETERMINANTS listed on any one of Tables 1-7 sufficient to measure a statistically significant alteration in PCDETERMINANT expression compared to a reference value.

Also provided by the present invention is a method for treating one or more subjects at risk for developing a metastatic tumor by detecting the presence of altered amounts of an effective amount of PCDETERMINANTS present in a sample from the one or more subjects; and treating the one or more subjects with one or more cancer-modulating drugs until altered amounts or activity of the PCDETERMINANTS return to a baseline value measured in one or more subjects at low risk for developing a metastatic disease, or alternatively, in subjects who do not exhibit any of the traditional risk factors for metastatic disease.

Also provided by the present invention is a method for treating one or more subjects having metastatic tumor by detecting the presence of altered levels of an effective amount of PCDETERMINANTS present in a sample from the one or more subjects; and treating the one or more subjects with one or more cancer-modulating drugs until altered amounts or activity of the PCDETERMINANTS return to a baseline value measured in one or more subjects at low risk for developing metastatic tumor.

Also provided by the present invention is a method for evaluating changes in the risk of developing metastatic prostate cancer in a subject diagnosed with cancer, by detecting an effective amount of PCDETERMINANTS (which may be two or more) in a first sample from the subject at a first period of time, detecting the amounts of the PCDETERMINANTS in a second sample from the subject at a second period of time, and comparing the amounts of the PCDETERMINANTS detected at the first and second periods of time.

Diagnostic and Prognostic Indications of the Invention

The invention allows the diagnosis and prognosis of a primary, locally invasive and/or metastatic tumor such as prostate, breast, among cancer types. The risk of developing metastatic prostate cancer can be detected by measuring an effective amount of PCDETERMINANT proteins, nucleic acids, polymorphisms, metabolites, and other analytes (which may be two or more) in a test sample (e.g., a subject derived sample), and comparing the effective amounts to reference or index values, often utilizing mathematical algorithms or formula in order to combine information from results of multiple individual PCDETERMINANTS and from non-analyte clinical parameters into a single measurement or index. Subjects identified as having an increased risk of a metastatic prostate cancer or other metastatic cancer types can optionally be selected to receive treatment regimens, such as administration of prophylactic or therapeutic compounds to prevent or delay the onset of metastatic prostate cancer or other metastatic cancer types.

The amount of the PCDETERMINANT protein, nucleic acid, polymorphism, metabolite, or other analyte can be measured in a test sample and compared to the “normal control level,” utilizing techniques such as reference limits, discrimination limits, or risk defining thresholds to define cutoff points and abnormal values. The “normal control level” means the level of one or more PCDETERMINANTS or combined PCDETERMINANT indices typically found in a subject not suffering from a metastatic tumor. Such normal control level and cutoff points may vary based on whether a PCDETERMINANT is used alone or in a formula combining with other PCDETERMINANTS into an index. Alternatively, the normal control level can be a database of PCDETERMINANT patterns from previously tested subjects who did not develop a metastatic tumor over a clinically relevant time horizon.

The present invention may be used to make continuous or categorical measurements of the risk of conversion to metastatic prostate cancer, or other metastatic cancer types thus diagnosing and defining the risk spectrum of a category of subjects defined as at risk for having a metastatic event. In the categorical scenario, the methods of the present invention can be used to discriminate between normal and disease subject cohorts. In other embodiments, the present invention may be used so as to discriminate those at risk for having a metastatic event from those having more rapidly progressing (or alternatively those with a shorter probable time horizon to a metastatic event) to a metastatic event from those more slowly progressing (or with a longer time horizon to a metastatic event), or those having metastatic cancer from normal. Such differing use may require different PCDETERMINANT combinations in individual panel, mathematical algorithm, and/or cut-off points, but be subject to the same aforementioned measurements of accuracy and other performance metrics relevant for the intended use.

Identifying the subject at risk of having a metastatic event enables the selection and initiation of various therapeutic interventions or treatment regimens in order to delay, reduce or prevent that subject's conversion to a metastatic disease state. Levels of an effective amount of PCDETERMINANT proteins, nucleic acids, polymorphisms, metabolites, or other analytes also allows for the course of treatment of a metastatic disease or metastatic event to be monitored. In this method, a biological sample can be provided from a subject undergoing treatment regimens, e.g., drug treatments, for cancer. If desired, biological samples are obtained from the subject at various time points before, during, or after treatment.

By virtue of some PCDETERMINANTS' being functionally active, by elucidating its function, subjects with high PCDETERMINANTS, for example, can be managed with agents/drugs that preferentially target such pathways, functioning through TGFβ signaling, thus, subjects can be treated with agents that enhance of block various components of the TGFβ signaling pathway.

The present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progression to conditions like cancer or metastatic events, will be of value in the operations of, for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein.

A machine-readable storage medium can comprise a data storage material encoded with machine readable data or data arrays which, when using a machine programmed with instructions for using said data, is capable of use for a variety of purposes, such as, without limitation, subject information relating to metastatic disease risk factors over time or in response drug therapies. Measurements of effective amounts of the biomarkers of the invention and/or the resulting evaluation of risk from those biomarkers can implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system of the invention may also be considered to be implemented as a computer-readable storage medium, configured with a computer program, where the storage medium so configured causes a computer to operate in a specific and predefined manner to perform various functions described herein.

Levels of an effective amount of PCDETERMINANT proteins, nucleic acids, polymorphisms, metabolites, or other analytes can then be determined and compared to a reference value, e.g. a control subject or population whose metastatic state is known or an index value or baseline value. The reference sample or index value or baseline value may be taken or derived from one or more subjects who have been exposed to the treatment, or may be taken or derived from one or more subjects who are at low risk of developing cancer or a metastatic event, or may be taken or derived from subjects who have shown improvements in as a result of exposure to treatment. Alternatively, the reference sample or index value or baseline value may be taken or derived from one or more subjects who have not been exposed to the treatment. For example, samples may be collected from subjects who have received initial treatment for caner or a metastatic event and subsequent treatment for cancer or a metastatic event to monitor the progress of the treatment. A reference value can also comprise a value derived from risk prediction algorithms or computed indices from population studies such as those disclosed herein.

The PCDETERMINANTS of the present invention can thus be used to generate a “reference PCDETERMINANT profile” of those subjects who do not have cancer or are not at risk of having a metastatic event, and would not be expected to develop cancer or a metastatic event. The PCDETERMINANTS disclosed herein can also be used to generate a “subject PCDETERMINANT profile” taken from subjects who have cancer or are at risk for having a metastatic event. The subject PCDETERMINANT profiles can be compared to a reference PCDETERMINANT profile to diagnose or identify subjects at risk for developing cancer or a metastatic event, to monitor the progression of disease, as well as the rate of progression of disease, and to monitor the effectiveness of treatment modalities. The reference and subject PCDETERMINANT profiles of the present invention can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

Differences in the genetic makeup of subjects can result in differences in their relative abilities to metabolize various drugs, which may modulate the symptoms or risk factors of cancer or metastatic events. Subjects that have cancer, or at risk for developing cancer or a metastatic event can vary in age, ethnicity, and other parameters. Accordingly, use of the PCDETERMINANTS disclosed herein, both alone and together in combination with known genetic factors for drug metabolism, allow for a pre-determined level of predictability that a putative therapeutic or prophylactic to be tested in a selected subject will be suitable for treating or preventing cancer or a metastatic event in the subject.

To identify therapeutics or drugs that are appropriate for a specific subject, a test sample from the subject can also be exposed to a therapeutic agent or a drug, and the level of one or more of PCDETERMINANT proteins, nucleic acids, polymorphisms, metabolites or other analytes can be determined. The level of one or more PCDETERMINANTS can be compared to sample derived from the subject before and after treatment or exposure to a therapeutic agent or a drug, or can be compared to samples derived from one or more subjects who have shown improvements in risk factors (e.g., clinical parameters or traditional laboratory risk factors) as a result of such treatment or exposure.

A subject cell (i.e., a cell isolated from a subject) can be incubated in the presence of a candidate agent and the pattern of PCDETERMINANT expression in the test sample is measured and compared to a reference profile, e.g., a metastatic disease reference expression profile or a non-disease reference expression profile or an index value or baseline value. The test agent can be any compound or composition or combination thereof, including, dietary supplements. For example, the test agents are agents frequently used in cancer treatment regimens and are described herein.

The aforementioned methods of the invention can be used to evaluate or monitor the progression and/or improvement of subjects who have been diagnosed with a cancer, and who have undergone surgical interventions.

Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between subjects having cancer, or at risk for cancer or a metastatic event, is based on whether the subjects have, a “significant alteration” (e.g., clinically significant “diagnostically significant) in the levels of a PCDETERMINANT. By “effective amount” it is meant that the measurement of an appropriate number of PCDETERMINANTS (which may be one or more) to produce a “significant alteration,” (e.g. level of expression or activity of a PCDETERMINANT) that is different than the predetermined cut-off point (or threshold value) for that PCDETERMINANT(S) and therefore indicates that the subject has cancer or is at risk for having a metastatic event for which the PCDETERMINANT(S) is a determinant. The difference in the level of PCDETERMINANT between normal and abnormal is preferably statistically significant. As noted below, and without any limitation of the invention, achieving statistical significance, and thus the preferred analytical, diagnostic, and clinical accuracy, generally but not always requires that combinations of several PCDETERMINANTS be used together in panels and combined with mathematical algorithms in order to achieve a statistically significant PCDETERMINANT index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

By predetermined level of predictability it is meant that the method provides an acceptable level of clinical or diagnostic accuracy. Using such statistics, an “acceptable degree of diagnostic accuracy”, is herein defined as a test or assay (such as the test of the invention for determining the clinically significant presence of PCDETERMINANTS, which thereby indicates the presence of cancer and/or a risk of having a metastatic event) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods predict the presence or absence of a cancer, metastatic cancer or response to therapy with at least 75% accuracy, more preferably 80%, 85%, 90%, 95%, 97%, 98%, 99% or greater accuracy.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing cancer or metastatic event, and the bottom quartile comprising the group of subjects having the lowest relative risk for developing cancer or a metastatic event. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease; such is the case with total cholesterol and for many inflammatory biomarkers with respect to their prediction of future metastatic events. Often such lower diagnostic accuracy tests must be combined with additional parameters in order to derive meaningful clinical thresholds for therapeutic intervention, as is done with the aforementioned global risk assessment indices.

A health economic utility function is an yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category (such as those attic risk for having a metastatic event) has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the PCDETERMINANTS of the invention allows for one of skill in the art to use the PCDETERMINANTS to identify, diagnose, or prognose subjects with a pre-determined level of predictability and performance.

Risk Markers of the Invention (PCDETERMINANTS)

The biomarkers and methods of the present invention allow one of skill in the art to identify, diagnose, or otherwise assess those subjects who do not exhibit any symptoms of cancer or a metastatic event, but who nonetheless may be at risk for developing cancer or a metastatic event.

We provides a murine mouse model for invasive and metastatic prostate cancer, where the mouse prostate epithelium sustains deletion of Pten and Smad4 gene. Table 1A comprises two hundred and eighty-four (284) overexpressed/amplified or downregulated/deleted genes. Table 1B comprises the three hundred and seventy-two (372) overexpressed/amplified or downregulated/deleted phenotype correlated human homologue PCDETERMINANTS of the present invention.

TABLE 1A
Gene Name

Up-Regulated Genes
Abl2: v-abl Abelson murine leukemia viral
oncogene 2 (arg, Abelson-related gene)
Actn1: actinin, alpha 1
Adam19: a disintegrin and metallopeptidase
domain 19 (meltrin beta)
Adam8: a disintegrin and metallopeptidase domain 8
Adamts12: a disintegrin-like and
metallopeptidase (reprolysin type) with
thrombospondin type 1 motif, 12
Adcy7: adenylate cyclase 7
Agtrl1: angiotensin receptor-like 1
Ak1: adenylate kinase 1
Aldh1a2: aldehyde dehydrogenase family 1, subfamily A2
Aldh1a3: aldehyde dehydrogenase family 1, subfamily A3
Angptl4: angiopoietin-like 4
Antxr2: anthrax toxin receptor 2
Arg1: arginase 1, liver
Axl: AXL receptor tyrosine kinase
B4galt5: UDP-Gal: betaGlcNAc beta 1,4-
galactosyltransferase, polypeptide 5
Bcl10: B-cell leukemia/lymphoma 10
Birc5: baculoviral IAP repeat-containing 5
Bmp1: bone morphogenetic protein 1
Bnip2: BCL2/adenovirus E1B interacting protein 1,NIP2
4632434I11Rik: RIKEN cDNA 4632434I11 gene
6330406I15Rik: RIKEN cDNA 6330406I15 gene
C1qb: complement component 1, q
subcomponent, beta polypeptide
1500015O10Rik: RIKEN cDNA 1500015O10 gene
1110032E23Rik: RIKEN cDNA 1110032E23 gene
Ccl20: chemokine (C-C motif) ligand 20
Ccnd1: cyclin D1
Ccnd2: cyclin D2
Ccr1: chemokine (C-C motif) receptor 1
Cd200: Cd200 antigen
Cd248: CD248 antigen, endosialin
Cd44: CD44 antigen
Cd53: CD53 antigen
Cd93: CD93 antigen
Cdc2a: cell division cycle 2 homolog A (S. pombe)
Cdca8: cell division cycle associated 8
Cdh11: cadherin 11
Cdkn2b: cyclin-dependent kinase inhibitor
2B (p15, inhibits CDK4)
Cebpb: CCAAT/enhancer binding protein (C/EBP), beta
Cenpa: centromere protein A
Chl1: cell adhesion molecule with homology to L1CAM
Chst11: carbohydrate sulfotransferase 11
Clec4n: C-type lectin domain family 4, member n
Clec7a: C-type lectin domain family 7, member a
Clic4: chloride intracellular channel 4 (mitochondrial)
Cnn2: calponin 2
Col10a1: procollagen, type X, alpha 1
Col12a1: procollagen, type XII, alpha 1
Col18a1: procollagen, type XVIII, alpha 1
Col1a1: procollagen, type I, alpha 1
Col1a2: procollagen, type I, alpha 2
Col3a1: procollagen, type III, alpha 1
Col4a1: procollagen, type IV, alpha 1
Col4a2: procollagen, type IV, alpha 2
Col5a1: procollagen, type V, alpha 1
Col5a2: procollagen, type V, alpha 2
Col8a1: procollagen, type VIII, alpha 1
Coro1a: coronin, actin binding protein 1A
Cotl1: coactosin-like 1 (Dictyostelium)
Cp: ceruloplasmin
Crlf1: cytokine receptor-like factor 1
Csrp1: cysteine and glycine-rich protein 1
Cthrc1: collagen triple helix repeat containing 1
Ctsz: cathepsin Z
Cxcl2: chemokine (C-X-C motif) ligand 2
Cxcl5: chemokine (C-X-C motif) ligand 5
Cxcr4: chemokine (C-X-C motif) receptor 4
Cybb: cytochrome b-245, beta polypeptide
Cyr61: cysteine rich protein 61
Ddah1: dimethylarginine dimethylaminohydrolase 1
Dpysl3: dihydropyrimidinase-like 3
Dsc2: desmocollin 2
Dusp4: dual specificity phosphatase 4
Dusp6: dual specificity phosphatase 6
1110006O17Rik: RIKEN cDNA 1110006O17 gene
Emilin2: elastin microfibril interfacer 2
Emp1: epithelial membrane protein 1
Endod1: endonuclease domain containing 1
Ets1: E26 avian leukemia oncogene 1, 5′ domain
Fbln2: fibulin 2
Fbn1: fibrillin 1
Fcer1g: Fc receptor, IgE, high affinity I, gamma polypeptide
Fcgr3: Fc receptor, IgG, low affinity III
Fcgr2b: Fc receptor, IgG, low affinity IIb
Fgf13: fibroblast growth factor 13
Fgfbp1: fibroblast growth factor binding protein 1
Fkbp10: FK506 binding protein 10
Flnb: Filamin, beta
Fn1: fibronectin 1
Fos: FBJ osteosarcoma oncogene
Frzb: frizzled-related protein
Fscn1: fascin homolog 1, actin bundling
protein (Strongylocentrotus purpuratus)
Fstl1: follistatin-like 1
Gatm: glycine amidinotransferase (L-
arginine: glycine amidinotransferase)
Gja1: gap junction membrane channel protein alpha 1
Gjb2: gap junction membrane channel protein beta 2
Glipr1: GLI pathogenesis-related 1 (glioma)
Gpm6b: glycoprotein m6b
Gpr124: G protein- coupled receptor 124
Gpx2: glutathione peroxidase 2
Hp: haptoglobin
Igf1: insulin-like growth factor 1
Igj: immunoglobulin joining chain
Il1b: interleukin 1 beta
Il4ra: interleukin 4 receptor, alpha
Inhbb: inhibin beta-B
Itgam: integrin alpha M
Itgax: integrin alpha X
Itgb2: integrin beta 2
Jag1: jagged 1
Jub: ajuba
2810417H13Rik: RIKEN cDNA 2810417H13 gene
Kpna3: karyopherin (importin) alpha 3
Krt14: keratin 14
Krt17: keratin 17
Krt5: keratin 5
Krt6a: keratin 6A
Lamb1-1: laminin B1 subunit 1
Lbh: limb-bud and heart
Lgals1: lectin, galactose binding, soluble 1
Lgals7: lectin, galactose binding, soluble 7
Lgmn: legumain
Lhfp: lipoma HMGIC fusion partner
Lox: lysyl oxidase
Loxl2: lysyl oxidase-like 2
Mcm5: minichromosome maintenance deficient 5,
cell division cycle 46 (S. cerevisiae)
Mmd: monocyte to macrophage
differentiation-associated
Mmp13: matrix metallopeptidase 13
Mmp14: matrix metallopeptidase 14 (membrane-inserted)
Mmp3: matrix metallopeptidase 3
Mrc2: mannose receptor, C type 2
Ms4a6b: membrane-spanning 4-domains, subfamily A, member 6B
Msn: moesin
Msrb3: methionine sulfoxide reductase B3
Myo1b: myosin IB
Nap1l1: nucleosome assembly protein 1-like 1
Ncf4: neutrophil cytosolic factor 4
Nid1: nidogen 1
Nrp1: neuropilin 1
Olfml2b: olfactomedin-like 2B
Osmr: oncostatin M receptor
Palld: palladin, cytoskeletal associated protein
Pcdh19: protocadherin 19
Pdgfb: platelet derived growth factor, B polypeptide
Pdgfrb: platelet derived growth factor receptor, beta polypeptide
Pdpn: podoplanin
Pla2g7: phospholipase A2, group VII
(platelet-activating factor acetylhydrolase, plasma)
Plek: pleckstrin
Plod2: procollagen lysine, 2-oxoglutarate 5-dioxygenase 2
Postn: periostin, osteoblast specific factor
Ppic: peptidylprolyl isomerase C
Ptgs2: prostaglandin-endoperoxide synthase 2
Ptprc: protein tyrosine phosphatase, receptor type, C
Pxdn: peroxidasin homolog (Drosophila)
Rbp1: retinol binding protein 1, cellular
Rftn1: raftlin lipid raft linker 1
Rgs4: regulator of G-protein signaling 4
C79267: expressed sequence C79267
Rrm2: ribonucleotide reductase M2
Serpine1: serine (or cysteine) peptidase inhibitor, clade E, member 1
Serpinf1: serine (or cysteine) peptidase inhibitor, clade F, member 1
Serpinh1: serine (or cysteine) peptidase inhibitor, clade H, member 1
Sfn: stratifin
Sfrp1: secreted frizzled-related sequence protein 1
Sh3pxd2b: SH3 and PX domains 2B
Slc15a3: solute carrier family 15, member 3
Slc16a1: solute carrier family 16
(monocarboxylic acid transporters), member 1
Slc20a1: solute carrier family 20, member 1
Slpi: secretory leukocyte peptidase inhibitor
Socs2: suppressor of cytokine signaling 2
Socs3: suppressor of cytokine signaling 3
Socs6: suppressor of cytokine signaling 6
Sparc: secreted acidic cysteine rich glycoprotein
Sfpi1: SFFV proviral integration 1
Spon1: spondin 1, (f-spondin) extracellular matrix protein
Spp1: secreted phosphoprotein 1
St3gal4: ST3 beta-galactoside alpha-2,3-sialyltransferase 4
Steap4: STEAP family member 4
Stom: stomatin
Svep1: sushi, von Willebrand factor type A,
EGF and pentraxin domain containing 1
Trf: transferrin
Tgfb3: transforming growth factor, beta 3
Tgfbi: transforming growth factor, beta induced
Tgfbr2: transforming growth factor, beta receptor II
Thbs2: thrombospondin 2
Timp1: tissue inhibitor of metalloproteinase 1
Timp3: tissue inhibitor of metalloproteinase 3
Tm4sf1: transmembrane 4 superfamily member 1
Tnc: tenascin C
Tnfaip2: tumor necrosis factor, alpha-induced protein 2
Tnfaip3: tumor necrosis factor, alpha-induced protein 3
Tnfrsf12a: tumor necrosis factor receptor superfamily, member 12a
Top2a: topoisomerase (DNA) II alpha
Tpm4: tropomyosin 4
Tubb6: tubulin, beta 6
Tyrobp: TYRO protein tyrosine kinase binding protein
Ube2c: ubiquitin-conjugating enzyme E2C
Uck2: uridine-cytidine kinase 2
Uhrf1: ubiquitin-like, containing PHD and
RING finger domains, 1
Vcl: vinculin
Vim: vimentin
Down-Regulated Genes
A4galt: alpha 1,4-galactosyltransferase
Abcc3: ATP-binding cassette, sub-family C (CFTR/MRP), member 3
Abcg5: ATP-binding cassette, sub-family G (WHITE), member 5
Abhd12: abhydrolase domain containing 12
Adh1: alcohol dehydrogenase 1 (class I)
Aldh1a1: aldehyde dehydrogenase family 1, subfamily A1
Anxa13: annexin A13
Ap1s3: adaptor-related protein complex AP-1, sigma 3
Arhgef4: Rho guanine nucleotide exchange factor (GEF) 4
Atoh1: atonal homolog 1 (Drosophila)
Atrn: attractin
AA986860: expressed sequence AA986860
2310007B03Rik: RIKEN cDNA 2310007B03 gene
Camk1d: calcium/calmodulin-dependent protein kinase ID
Capn13: calpain 13
Chka: choline kinase alpha
Crym: crystallin, mu
Ctse: cathepsin E
Cyb5b: cytochrome b5 type B
Degs2: degenerative spermatocyte homolog
2 (Drosophila), lipid desaturase
Dgat2: diacylglycerol O-acyltransferase 2
Epb4.1l4b: erythrocyte protein band 4.1-like 4b
Fmo2: flavin containing monooxygenase 2
Fmo3: flavin containing monooxygenase 3
Gata2: GATA binding protein 2
Gata3: GATA binding protein 3
Gpld1: glycosylphosphatidylinositol specific phospholipase D1
Gsn: gelsolin
Gsto1: glutathione S-transferase omega 1
Hmgcs2: 3-hydroxy-3-methylglutaryl-Coenzyme A synthase 2
Hmgn3: high mobility group nucleosomal binding domain 3
Hpgd: hydroxyprostaglandin dehydrogenase 15 (NAD)
4632417N05Rik: RIKEN cDNA 4632417N05 gene
Id1: inhibitor of DNA binding 1
Id2: inhibitor of DNA binding 2
Id3: inhibitor of DNA binding 3
Id4: inhibitor of DNA binding 4
Ihh: Indian hedgehog
Iqgap2: IQ motif containing GTPase activating protein 2
Kbtbd11: kelch repeat and BTB (POZ) domain containing 11
2310057J16Rik: RIKEN cDNA 2310057J16 gene
Krt15: keratin 15
Krt4: keratin 4
Ltb4dh: leukotriene B4 12-hydroxydehydrogenase
Mal: myelin and lymphocyte protein, T-cell differentiation protein
Mettl7a: methyltransferase like 7A
Mid1: midline 1
AA536749: Expressed sequence AA536749
Ms4a8a: membrane-spanning 4-domains, subfamily A, member 8A
Ncoa4: nuclear receptor coactivator 4
Nnat: neuronatin
Padi1: peptidyl arginine deiminase, type I
Papss2: 3′-phosphoadenosine 5′-phosphosulfate synthase 2
Pdk2: pyravate dehydrogenase kinase, isoenzyme 2
Pfn2: profilin 2
Pink1: PTEN induced putative kinase 1
Pllp: plasma membrane proteolipid
Pparg: peroxisome proliferator activated receptor gamma
Psca: prostate stem cell antigen
Ptgs1: prostaglandin-endoperoxide synthase 1
Rab17: RAB17, member RAS oncogene family
Rab27b: RAB27b, member RAS oncogene family
Gm106: gene model 106, (NCBI)
Rtn4rl1: reticulon 4 receptor-like 1
Scnn1a: sodium channel, nonvoltage-gated, type I, alpha
Slc12a7: solute carrier family 12, member 7
Sord: sorbitol dehydrogenase
Sprr2a: small proline-rich protein 2A
Stard10: START domain containing 10
Stat5a: signal transducer and activator of transcription 5A
Tbx3: T-box 3
Tesc: tescalcin
Tff3: trefoil factor 3, intestinal
Timp4: tissue inhibitor of metalloproteinase 4
Tmem159: transmembrane protein 159
Tmem45b: transmembrane protein 45b
Trim2: tripartite motif protein 2
Tspan8: tetraspanin 8
Ttr: transthyretin
Ugt2b35: UDP glucuronosyltransferase 2 family, polypeptide B35
Upk1a: uroplakin 1A
Upk1b: uroplakin 1B
Zbtb16: zinc finger and BTB domain containing 16
Zdhhc14: zinc finger, DHHC domain containing 14

TABLE 1B
PC PCDETERMINANTS (372 genes)

| | | Fold | |
|---|---|---|---|
| | | Change in | PCDeterminant |
| Name | Description | Expresion | No: |
| Up-Regulated Genes | | | |
| ABL2 | Abl2: v-abl Abelson murine leukemia viral oncogene 2 | 2.73 | 1 |
| | (arg, Abelson-related gene) | | |
| ACTN1 | Actn1: actinin, alpha 1 | 2.01 | 2 |
| ADAM19 | Adam19: a disintegrin and metallopeptidase domain 19 | 2.69 | 3 |
| | (meltrin beta) | | |
| ADAM8 | Adam8: a disintegrin and metallopeptidase domain 8 | 2.42 | 4 |
| ADAMTS12 | Adamts12: a disintegrin-like and metallopeptidase | 4.84 | 5 |
| | (reprolysin type) with thrombospondin type 1 motif, 12 | | |
| ADCY7 | Adcy7: adenylate cyclase 7 | 2.75 | 6 |
| AGTRL1 | Agtrl1: angiotensin receptor-like 1 | 3.25 | 7 |
| AK1 | Ak1: adenylate kinase 1 | 2.47 | 8 |
| ALDH1A2 | Aldh1a2: aldehyde dehydrogenase family 1, subfamily A2 | 3.62 | 9 |
| ALDH1A3 | Aldh1a3: aldehyde dehydrogenase family 1, subfamily A3 | 10.58 | 10 |
| ANGPTL4 | Angptl4: angiopoietin-like 4 | 8.58 | 11 |
| ANTXR2 | Antxr2: anthrax toxin receptor 2 | 2.59 | 12 |
| ARG1 | Arg1: arginase 1, liver | 3.08 | 13 |
| AXL | Axl: AXL receptor tyrosine kinase | 2.27 | 14 |
| B4GALT5 | B4galt5: UDP-Gal: betaGlcNAc beta 1,4- | 2.69 | 15 |
| | galactosyltransferase, polypeptide 5 | | |
| BCL10 | Bcl10: B-cell leukemia/lymphoma 10 | 2.10 | 16 |
| BIRC5 | Birc5: baculoviral IAP repeat-containing 5 | 2.99 | 17 |
| BMP1 | Bmp1: bone morphogenetic protein 1 | 2.46 | 18 |
| BNC1 | basonuclin 1 | 3.383 | 19 |
| BNIP2 | Bnip2: BCL2/adenovirus E1B interacting protein 1, NIP2 | 2.71 | 20 |
| BRCA1 | breast cancer 1, early onset | 3.225 | 21 |
| BST1 | bone marrow stromal cell antigen 1 | 4.903 | 22 |
| C11orf82 | 4632434I11Rik: RIKEN cDNA 4632434I11 gene | 4.49 | 23 |
| C13orf33 | 6330406I15Rik: RIKEN cDNA 6330406I15 gene | 3.15 | 24 |
| C1QB | C1qb: complement component 1, q subcomponent, | 2.31 | 25 |
| | beta polypeptide | | |
| C2orf40 | 1500015O10Rik: RIKEN cDNA 1500015O10 gene | 6.79 | 26 |
| C4orf18 | 1110032E23Rik: RIKEN cDNA 1110032E23 gene | 3.14 | 27 |
| CCDC99 | coiled-coil domain containing 99 | 4.627 | 28 |
| CCL2 | chemokine (C-C motif) ligand 2 | 2.107 | 29 |
| CCL20 | Ccl20: chemokine (C-C motif) ligand 20 | 10.18 | 30 |
| CCND1 | Ccnd1: cyclin D1 | 2.43 | 31 |
| CCND2 | Ccnd2: cyclin D2 | 3.13 | 32 |
| CCR1 | Ccr1: chemokine (C-C motif) receptor 1 | 3.59 | 33 |
| CD200 | Cd200: Cd200 antigen | 2.20 | 34 |
| CD248 | Cd248: CD248 antigen, endosialin | 2.34 | 35 |
| CD44 | Cd44: CD44 antigen | 2.94 | 36 |
| CD53 | Cd53: CD53 antigen | 2.59 | 37 |
| CD93 | Cd93: CD93 antigen | 2.59 | 38 |
| CDC2 | Cdc2a: cell division cycle 2 homolog A (S. pombe) | 2.87 | 39 |
| CDCA2 | cell division cycle associated 2 | 4.298 | 40 |
| CDCA8 | Cdca8: cell division cycle associated 8 | 3.43 | 41 |
| CDH11 | Cdh11: cadherin 11 | 4.24 | 42 |
| CDKN2B | Cdkn2b: cyclin-dependent kinase inhibitor 2B (p15, | 3.14 | 43 |
| | inhibits CDK4) | | |
| CEBPB | Cebpb: CCAAT/enhancer binding protein (C/EBP), beta | 2.43 | 44 |
| CENPA | Cenpa: centromere protein A | 2.90 | 45 |
| CEP55 | centrosomal protein 55 kDa | 2.268 | 46 |
| CHL1 | Chl1: cell adhesion molecule with homology to L1CAM | 5.68 | 47 |
| CHST11 | Chst11: carbohydrate sulfotransferase 11 | 3.55 | 48 |
| CLEC6A | Clec4n: C-type lectin domain family 4, member n | 4.28 | 49 |
| Clec7a | Clec7a: C-type lectin domain family 7, member a | 2.37 | 50 |
| CLIC4 | Clic4: chloride intracellular channel 4 (mitochondrial) | 2.06 | 51 |
| CNN2 | Cnn2: calponin 2 | 2.49 | 52 |
| COL10A1 | Col10a1: procollagen, type X, alpha 1 | 32.71 | 53 |
| COL12A1 | Col12a1: procollagen, type XII, alpha 1 | 5.19 | 54 |
| COL18A1 | Col18a1: procollagen, type XVIII, alpha 1 | 3.31 | 55 |
| COL1A1 | Col1a1: procollagen, type I, alpha 1 | 4.56 | 56 |
| COL1A2 | Col1a2: procollagen, type I, alpha 2 | 3.48 | 57 |
| COL3A1 | Col3a1: procollagen, type III, alpha 1 | 3.75 | 58 |
| COL4A1 | Col4a1: procollagen, type IV, alpha 1 | 3.69 | 59 |
| COL4A2 | Col4a2: procollagen, type IV, alpha 2 | 3.07 | 60 |
| COL5A1 | Col5a1: procollagen, type V, alpha 1 | 3.98 | 61 |
| COL5A2 | Col5a2: procollagen, type V, alpha 2 | 5.19 | 62 |
| COL5A3 | collagen, type V, alpha 3 | 2.169 | 63 |
| COL8A1 | Col8a1: procollagen, type VIII, alpha 1 | 5.26 | 64 |
| CORO1A | Coro1a: coronin, actin binding protein 1A | 3.14 | 65 |
| COTL1 | Cotl1: coactosin-like 1 (Dictyostelium) | 2.01 | 66 |
| CP | Cp: ceruloplasmin | 4.66 | 67 |
| CRH | corticotropin releasing hormone | 11.092 | 68 |
| CRLF1 | Crlf1: cytokine receptor-like factor 1 | 5.47 | 69 |
| CSF2RB | colony stimulating factor 2 receptor, beta, low- | 3.114 | 70 |
| | affinity (granulocyte-macrophage) | | |
| CSRP1 | Csrp1: cysteine and glycine-rich protein 1 | 2.16 | 71 |
| CTHRC1 | Cthrc1: collagen triple helix repeat containing 1 | 7.81 | 72 |
| CTSZ | Ctsz: cathepsin Z | 2.11 | 73 |
| CXCL1 | chemokine (C-X-C motif) ligand 1 (melanoma | 4.704 | 74 |
| | growth stimulating activity, alpha) | | |
| CXCL2 | chemokine (C-X-C motif) ligand 2 | 5.666 | 75 |
| CXCL3 | Cxcl2: chemokine (C-X-C motif) ligand 2 | 13.11 | 76 |
| CXCL6 | Cxcl5: chemokine (C-X-C motif) ligand 5 | 11.02 | 77 |
| CXCR4 | Cxcr4: chemokine (C-X-C motif) receptor 4 | 3.19 | 78 |
| CYBB | Cybb: cytochrome b-245, beta polypeptide | 2.03 | 79 |
| CYP7B1 | cytochrome P450, family 7, subfamily B, polypeptide 1 | 4.543 | 80 |
| CYR61 | Cyr61: cysteine rich protein 61 | 3.68 | 81 |
| DDAH1 | Ddah1: dimethylarginine dimethylaminohydrolase 1 | 4.10 | 82 |
| DMBX1 | diencephalon/mesencephalon homeobox 1 | 3.067 | 83 |
| DPYSL3 | Dpys13: dihydropyrimidinase-like 3 | 2.69 | 84 |
| DSC2 | Dsc2: desmocollin 2 | 2.19 | 85 |
| DSC3 | desmocollin 3 | 2.319 | 86 |
| DUSP4 | Dusp4: dual specificity phosphatase 4 | 6.26 | 87 |
| DUSP6 | Dusp6: dual specificity phosphatase 6 | 4.42 | 88 |
| ECSM2 | 1110006O17Rik: RIKEN cDNA 1110006O17 gene | 2.36 | 89 |
| EMILIN2 | Emilin2: elastin microfibril interfacer 2 | 2.37 | 90 |
| EMP1 | Emp1: epithelial membrane protein 1 | 2.21 | 91 |
| ENDOD1 | Endod1: endonuclease domain containing 1 | 2.52 | 92 |
| ETS1 | Ets1: E26 avian leukemia oncogene 1, 5′ domain | 2.46 | 93 |
| FAP | fibroblast activation protein, alpha | 3.121 | 94 |
| FBLN2 | Fbln2: fibulin 2 | 3.16 | 95 |
| FBN1 | Fbn1: fibrillin 1 | 3.65 | 96 |
| FCER1G | Fcer1g: Fc receptor, IgE, high affinity I, gamma | 2.14 | 97 |
| | polypeptide | | |
| FCGR2A | Fcgr3: Fc receptor, IgG, low affinity III | 2.02 | 98 |
| FCGR2B | Fcgr2b: Fc receptor, IgG, low affinity IIb | 3.63 | 99 |
| FERMT3 | fermitin family homolog 3 (Drosophila) | 2.338 | 100 |
| FGF13 | Fgf13: fibroblast growth factor 13 | 3.14 | 101 |
| FGFBP1 | Fgfbp1: fibroblast growth factor binding protein 1 | 2.87 | 102 |
| FKBP10 | Fkbp10: FK506 binding protein 10 | 4.85 | 103 |
| FLNB | Flnb: Filamin, beta | 2.10 | 104 |
| FN1 | Fn1: fibronectin 1 | 5.01 | 105 |
| FOS | Fos: FBJ osteosarcoma oncogene | 2.57 | 106 |
| FPR2 | formyl peptide receptor 2 | 7.272 | 107 |
| FRZB | Frzb: frizzled-related protein | 4.30 | 108 |
| FSCN1 | Fscn1: fascin homolog 1, actin bundling protein | 7.57 | 109 |
| | (Strongylocentrotus purpuratus) | | |
| FSTL1 | Fstl1: follistatin-like 1 | 2.87 | 110 |
| FSTL3 | follistatin-like 3 (secreted glycoprotein) | 6.314 | 111 |
| GATM | Gatm: glycine amidinotransferase (L-arginine: glycine | 2.23 | 112 |
| | amidinotransferase) | | |
| GCNT2 | glucosaminyl (N-acetyl) transferase 2, I-branching | 2.049 | 113 |
| | enzyme (I blood group) | | |
| GJA1 | Gja1: gap junction membrane channel protein alpha 1 | 3.67 | 114 |
| GJB2 | Gjb2: gap junction membrane channel protein beta 2 | 2.35 | 115 |
| GLIPR1 | Glipr1: GLI pathogenesis-related 1 (glioma) | 2.29 | 116 |
| GPM6B | Gpm6b: glycoprotein m6b | 2.16 | 117 |
| GPR124 | Gpr124: G protein-coupled receptor 124 | 2.51 | 118 |
| GPX2 | Gpx2: glutathione peroxidase 2 | 3.70 | 119 |
| HMGB2 | high-mobility group box 2 | 2.024 | 120 |
| HPR | Hp: haptoglobin | 10.62 | 121 |
| ICAM1 | intercellular adhesion molecule 1 | 2.594 | 122 |
| IDI1 | isopentenyl-diphosphate delta isomerase 1 | 2.528 | 123 |
| IGF1 | Igf1: insulin-like growth factor 1 | 2.37 | 124 |
| IGJ | Igj: immunoglobulin joining chain | 4.44 | 125 |
| IL1B | Il1b: interleukin 1 beta | 3.94 | 126 |
| IL1RAP | interleukin 1 receptor accessory protein | 3.072 | 127 |
| IL4R | Il4ra: interleukin 4 receptor, alpha | 3.04 | 128 |
| INHBB | Inhbb: inhibin beta-B | 3.72 | 129 |
| ITGAM | Itgam: integrin alpha M | 4.09 | 130 |
| ITGAX | Itgax: integrin alpha X | 4.25 | 131 |
| ITGB2 | Itgb2: integrin beta 2 | 2.78 | 132 |
| JAG1 | Jag1: jagged 1 | 2.64 | 133 |
| JUB | Jub: ajuba | 2.27 | 134 |
| KIAA0101 | 2810417H13Rik: RIKEN cDNA 2810417H13 gene | 3.30 | 135 |
| KIF22 | kinesin family member 22 | 2.257 | 136 |
| KLHL6 | kelch-like 6 (Drosophila) | 4.358 | 137 |
| KLK7 | kallikrein-related peptidase 7 | 7.652 | 138 |
| KPNA3 | Kpna3: karyopherin (importin) alpha 3 | 2.13 | 139 |
| KRT14 | Krt14: keratin 14 | 8.90 | 140 |
| KRT17 | Krt17: keratin 17 | 18.65 | 141 |
| KRT5 | Krt5: keratin 5 | 2.53 | 142 |
| KRT6A | Krt6a: keratin 6A | 13.37 | 143 |
| LAMB1 | Lamb1-1: laminin B1 subunit 1 | 2.28 | 144 |
| LBH | Lbh: limb-bud and heart | 5.00 | 145 |
| LGALS1 | Lgals1: lectin, galactose binding, soluble 1 | 3.55 | 146 |
| LGALS7 | Lgals7: lectin, galactose binding, soluble 7 | 2.35 | 147 |
| LGMN | Lgmn: legumain | 2.32 | 148 |
| LHFP | Lhfp: lipoma HMGIC fusion partner | 3.03 | 149 |
| LOX | Lox: lysyl oxidase | 3.74 | 150 |
| LOXL2 | Loxl2: lysyl oxidase-like 2 | 3.96 | 151 |
| LRIG1 | leucine-rich repeats and immunoglobulin-like | 5.601 | 152 |
| | domains 1 | | |
| MAP3K8 | mitogen-activated protein kinase kinase kinase 8 | 2.454 | 153 |
| MCM5 | Mcm5: minichromosome maintenance deficient 5, cell | 2.48 | 154 |
| | division cycle 46 (S. cerevisiae) | | |
| MCM6 | minichromosome maintenance complex component 6 | 2.596 | 155 |
| MKI67 | antigen identified by monoclonal antibody Ki-67 | 2.024 | 156 |
| MMD | Mmd: monocyte to macrophage differentiation- | 2.01 | 157 |
| | associated | | |
| MMP13 | Mmp13: matrix metallopeptidase 13 | 20.59 | 158 |
| MMP14 | Mmp14: matrix metallopeptidase 14 (membrane- | 2.09 | 159 |
| | inserted) | | |
| MMP3 | Mmp3: matrix metallopeptidase 3 | 11.48 | 160 |
| MRC2 | Mrc2: mannose receptor, C type 2 | 4.01 | 161 |
| MS4A6A | Ms4a6b: membrane-spanning 4-domains, subfamily A, | 2.23 | 162 |
| | member 6B | | |
| MSN | Msn: moesin | 3.44 | 163 |
| MSRB3 | Msrb3: methionine sulfoxide reductase B3 | 2.28 | 164 |
| MYO1B | Myo1b: myosin IB | 2.32 | 165 |
| NAP1L1 | Nap1l1: nucleosome assembly protein 1-like 1 | 2.08 | 166 |
| NCF1 | neutrophil cytosolic factor 1 | 2.218 | 167 |
| NCF4 | Ncf4: neutrophil cytosolic factor 4 | 3.51 | 168 |
| NID1 | Nid1: nidogen 1 | 2.26 | 169 |
| NKD2 | naked cuticle homolog 2 (Drosophila) | 2.027 | 170 |
| NRP1 | Nrp1: neuropilin 1 | 2.63 | 171 |
| OLFML2B | Olfml2b: olfactomedin-like 2B | 9.97 | 172 |
| OSMR | Osmr: oncostatin M receptor | 3.05 | 173 |
| PALLD | Palld: palladin, cytoskeletal associated protein | 2.23 | 174 |
| PCDH19 | Pcdh19: protocadherin 19 | 2.65 | 175 |
| PDGFB | Pdgfb: platelet derived growth factor, B polypeptide | 2.99 | 176 |
| PDGFRB | Pdgfrb: platelet derived growth factor receptor, beta | 4.45 | 177 |
| | polypeptide | | |
| PDPN | Pdpn: podoplanin | 2.50 | 178 |
| PLA2G7 | Pla2g7: phospholipase A2, group VII (platelet- | 4.76 | 179 |
| | activating factor acetylhydrolase, plasma) | | |
| PLEK | Plek: pleckstrin | 2.95 | 180 |
| PLOD2 | Plod2: procollagen lysine, 2-oxoglutarate 5- | 2.74 | 181 |
| | dioxygenase 2 | | |
| POSTN | Postn: periostin, osteoblast specific factor | 5.24 | 182 |
| PPIC | Ppic: peptidylprolyl isomerase C | 2.99 | 183 |
| PTGS2 | Ptgs2: prostaglandin-endoperoxide synthase 2 | 14.78 | 184 |
| PTPRC | Ptprc: protein tyrosine phosphatase, receptor type, C | 2.88 | 185 |
| PXDN | Pxdn: peroxidasin homolog (Drosophila) | 4.76 | 186 |
| RBP1 | Rbp1: retinol binding protein 1, cellular | 2.59 | 187 |
| RFTN1 | Rftn1: raftlin lipid raft linker 1 | 3.20 | 188 |
| RGS16 | regulator of G-protein signaling 16 | 14.021 | 189 |
| RGS4 | Rgs4: regulator of G-protein signaling 4 | 21.97 | 190 |
| RP1-93P18.1 | C79267: expressed sequence C79267 | 7.21 | 191 |
| RRM2 | Rrm2: ribonucleotide reductase M2 | 2.77 | 192 |
| SAA1 | serum amyloid A1 | 5.722 | 193 |
| SERPINE1 | Serpine1: serine (or cysteine) peptidase inhibitor, clade | 5.56 | 194 |
| | E, member 1 | | |
| SERPINF1 | Serpinf1: serine (or cysteine) peptidase inhibitor, clade | 2.44 | 195 |
| | F, member 1 | | |
| SERPINH1 | Serpinh1: serine (or cysteine) peptidase inhibitor, clade | 3.83 | 196 |
| | H, member 1 | | |
| SFN | Sfn: stratifin | 4.34 | 197 |
| SFRP1 | Sfrp1: secreted frizzled-related sequence protein 1 | 3.15 | 198 |
| SH3PXD2B | Sh3pxd2b: SH3 and PX domains 2B | 2.47 | 199 |
| SLC15A3 | Slc15a3: solute carrier family 15, member 3 | 3.02 | 200 |
| SLC16A1 | Slc16a1: solute carrier family 16 (monocarboxylic acid | 5.13 | 201 |
| | transporters), member 1 | | |
| SLC20A1 | Slc20a1: solute carrier family 20, member 1 | 2.76 | 202 |
| SLC5A8 | solute carrier family 5 (iodide transporter), member 8 | 3.799 | 203 |
| SLC5A9 | solute carrier family 5 (sodium/glucose | 4.382 | 204 |
| | cotransporter), member 9 | | |
| SLPI | Slpi: secretory leukocyte peptidase inhibitor | 4.74 | 205 |
| SOCS2 | Socs2: suppressor of cytokine signaling 2 | 2.22 | 206 |
| SOCS3 | Socs3: suppressor of cytokine signaling 3 | 3.51 | 207 |
| SOCS6 | Socs6: suppressor of cytokine signaling 6 | 2.20 | 208 |
| SPARC | Spare: secreted acidic cysteine rich glycoprotein | 3.97 | 209 |
| SPI1 | Sfpi1: SFFV proviral integration 1 | 2.49 | 210 |
| SPON1 | Spon1: spondin 1, (f-spondin) extracellular matrix | 8.24 | 211 |
| | protein | | |
| SPP1 | Spp1: secreted phosphoprotein 1 | 23.53 | 212 |
| ST3GAL4 | St3gal4: ST3 beta-galactoside alpha-2,3- | 2.93 | 213 |
| | sialyltransferase 4 | | |
| STEAP3 | STEAP family member 3 | 3.367 | 214 |
| STEAP4 | Steap4: STEAP family member 4 | 2.31 | 215 |
| STOM | Stom: stomatin | 2.21 | 216 |
| SVEP1 | Svep1: sushi, von Willebrand factor type A, EGF and | 3.04 | 217 |
| | pentraxin domain containing 1 | | |
| TF | Trf: transferrin | 4.57 | 218 |
| TGFB3 | Tgfb3: transforming growth factor, beta 3 | 2.64 | 219 |
| TGFBI | Tgfbi: transforming growth factor, beta induced | 5.70 | 220 |
| TGFBR2 | Tgfbr2: transforming growth factor, beta receptor II | 4.91 | 221 |
| THBS1 | thrombospondin 1 | 4.036 | 222 |
| THBS2 | Thbs2: thrombospondin 2 | 9.19 | 223 |
| TIMP1 | Timp1: tissue inhibitor of metalloproteinase 1 | 4.27 | 224 |
| TIMP3 | Timp3: tissue inhibitor of metalloproteinase 3 | 2.06 | 225 |
| TM4SF1 | Tm4sf1: transmembrane 4 superfamily member 1 | 5.35 | 226 |
| TNC | Tnc: tenascin C | 11.41 | 227 |
| TNF | tumor necrosis factor (TNF superfamily, member 2) | 3.124 | 228 |
| TNFAIP2 | Tnfaip2: tumor necrosis factor, alpha-induced protein 2 | 3.32 | 229 |
| TNFAIP3 | Tnfaip3: tumor necrosis factor, alpha-induced protein 3 | 2.69 | 230 |
| TNFAIP8L2 | tumor necrosis factor, alpha-induced protein 8-like 2 | 3.879 | 231 |
| TNFRSF12A | Tnfrsf12a: tumor necrosis factor receptor superfamily, | 2.76 | 232 |
| | member 12a | | |
| TOP2A | Top2a: topoisomerase (DNA) II alpha | 2.16 | 233 |
| TPM4 | Tpm4: tropomyosin 4 | 2.71 | 234 |
| TTC9 | tetratricopeptide repeat domain 9 | 7.031 | 235 |
| TUBB6 | Tubb6: tubulin, beta 6 | 4.24 | 236 |
| TYROBP | Tyrobp: TYRO protein tyrosine kinase binding protein | 2.65 | 237 |
| UBE2C | Ube2c: ubiquitin-conjugating enzyme E2C | 3.45 | 238 |
| UCK2 | Uck2: uridine-cytidine kinase 2 | 2.33 | 239 |
| UHRF1 | Uhrf1: ubiquitin-like, containing PHD and RING finger | 3.85 | 240 |
| | domains, 1 | | |
| VCAN | versican | 3.006 | 241 |
| VCL | Vcl: vinculin | 2.60 | 242 |
| VIM | Vim: vimentin | 2.44 | 243 |
| WISP1 | WNT1 inducible signaling pathway protein 1 | 7.770 | 244 |
| ZEB2 | zinc finger E-box binding homeobox 2 | 2.832 | 245 |
| Down-Regulated Genes | | | |
| A4GALT | A4galt: alpha 1,4-galactosyltransferase | −4.445274 | 246 |
| ABCA5 | ATP-binding cassette, sub-family A (ABC1), | −2.306 | 247 |
| | member 5 | | |
| ABCC3 | Abcc3: ATP-binding cassette, sub-family C | −2.434092 | 248 |
| | (CFTR/MRP), member 3 | | |
| ABCG5 | Abcg5: ATP-binding cassette, sub-family G (WHITE), | −8.156716 | 249 |
| | member 5 | | |
| ABHD12 | Abhd12: abhydrolase domain containing 12 | −2.824131 | 250 |
| ADH1C | Adh1: alcohol dehydrogenase 1 (class I) | −3.563348 | 251 |
| AHCYL2 | S-adenosylhomocysteine hydrolase-like 2 | −2.142 | 252 |
| ALDH1A1 | Aldh1a1: aldehyde dehydrogenase family 1, subfamily A1 | −3.198218 | 253 |
| ANXA13 | Anxa13: annexin A13 | −2.689684 | 254 |
| AP1S3 | Ap1s3: adaptor-related protein complex AP-1, sigma 3 | −4.036778 | 255 |
| ARHGEF4 | Arhgef4: Rho guanine nucleotide exchange factor (GEF) 4 | −2.231166 | 256 |
| ATOH1 | Atoh1: atonal homolog 1 (Drosophila) | −3.063348 | 257 |
| ATP6V1C2 | ATPase, H+ transporting, lysosomal 42 kDa, V1 | −7.509 | 258 |
| | subunit C2 | | |
| ATRN | Atrn: attractin | −2.669374 | 259 |
| BEST2 | bestrophin 2 | −19.994 | 260 |
| BEX4 | brain expressed, X-linked 4 | −3.94 | 261 |
| BMP15 | bone morphogenetic protein 15 | −6.201 | 262 |
| C1orf116 | AA986860: expressed sequence AA986860 | −2.311741 | 263 |
| C2orf54 | 2310007B03Rik: RIKEN cDNA 2310007B03 gene | −2.42381 | 264 |
| CAMK1D | Camk1d: calcium/calmodulin-dependent protein kinase ID | −2.303511 | 265 |
| CAPN13 | Capn13: calpain 13 | −2.458414 | 266 |
| CHKA | Chka: choline kinase alpha | −2.592185 | 267 |
| CLDN8 | claudin 8 | −2.234 | 268 |
| CRYM | Crym: crystallin, mu | −4.068841 | 269 |
| CTSE | Ctse: cathepsin E | −4.859607 | 270 |
| CYB5B | Cyb5b: cytochrome b5 type B | −2.48918 | 271 |
| DEGS2 | Degs2: degenerative spermatocyte homolog 2 | −3.330377 | 272 |
| | (Drosophila), lipid desaturase | | |
| DGAT2 | Dgat2: diacylglycerol O-acyltransferase 2 | −2.217621 | 273 |
| DNPEP | aspartyl aminopeptidase | −2.009 | 274 |
| EPB41L4B | Epb4.1l4b: erythrocyte protein band 4.1-like 4b | −2.840452 | 275 |
| EPS8L3 | EPS8-like 3 | −2.465 | 276 |
| FMO2 | Fmo2: flavin containing monooxygenase 2 | −2.195393 | 277 |
| FMO3 | Fmo3: flavin containing monooxygenase 3 | −4.598326 | 278 |
| FMOD | fibromodulin | −2.332 | 279 |
| FOXQ1 | forkhead box Q1 | −2.224 | 280 |
| GATA2 | Gata2: GATA binding protein 2 | −2.734637 | 281 |
| GATA3 | Gata3: GATA binding protein 3 | −2.699067 | 282 |
| GLB1L2 | galactosidase, beta 1-like 2 | −4.154 | 283 |
| GPLD1 | Gpld1: glycosylphosphatidylinositol specific | −2.639069 | 284 |
| | phospholipase D1 | | |
| GSN | Gsn: gelsolin | −2.747031 | 285 |
| GSTM5 | glutathione S-transferase mu 5 | −2.062 | 286 |
| GSTO1 | Gstol: glutathione S-transferase omega 1 | −2.043964 | 287 |
| HDAC11 | histone deacetylase 11 | −2.077 | 288 |
| HMGCS2 | Hmgcs2: 3-hydroxy-3-methylglutaryl-Coenzyme A | −9.204545 | 289 |
| | synthase 2 | | |
| HMGN3 | Hmgn3: high mobility group nucleosomal binding | −4.078795 | 290 |
| | domain 3 | | |
| HPGD | Hpgd: hydroxyprostaglandin dehydrogenase 15 (NAD) | −3.769384 | 291 |
| HSD11B2 | hydroxysteroid (11-beta) dehydrogenase 2 | −4.061 | 292 |
| HSPC105 | 4632417N05Rik: RIKEN cDNA 4632417N05 gene | −2.404494 | 293 |
| ID1 | Id1: inhibitor of DNA binding 1 | −7.414017 | 294 |
| ID2 | Id2: inhibitor of DNA binding 2 | −2.378587 | 295 |
| ID3 | Id3: inhibitor of DNA binding 3 | −4.716649 | 296 |
| ID4 | Id4: inhibitor of DNA binding 4 | −2.177835 | 297 |
| IHH | Ihh: Indian hedgehog | −10.58065 | 298 |
| IQGAP2 | Iqgap2: IQ motif containing GTPase activating protein 2 | −2.998478 | 299 |
| KBTBD11 | Kbtbd11: kelch repeat and BTB (POZ) domain | −2.23538 | 300 |
| | containing 11 | | |
| KIAA1543 | 2310057J16Rik: RIKEN cDNA 2310057J16 gene | −2.32299 | 301 |
| KRT15 | Krt15: keratin 15 | −2.63679 | 302 |
| KRT4 | Krt4: keratin 4 | −2.228175 | 303 |
| KRT78 | keratin 78 | −2.88 | 304 |
| LASS4 | LAG1 homolog, ceramide synthase 4 | −2.836 | 305 |
| LPHN1 | latrophilin 1 | −2.412 | 306 |
| LTB4DH | Ltb4dh: leukotriene B4 12-hydroxydehydrogenase | −2.383255 | 307 |
| LY6K | lymphocyte antigen 6 complex, locus K | −5.539 | 308 |
| MAL | Mal: myelin and lymphocyte protein, T-cell | −2.911572 | 309 |
| | differentiation protein | | |
| METTL7A | Mettl7a: methyltransferase like 7A | −2.749635 | 310 |
| MID1 | Mid1: midline 1 | −3.369582 | 311 |
| M-RIP | AA536749: Expressed sequence AA536749 | −2.086553 | 312 |
| MS4A8B | Ms4a8a: membrane-spanning 4-domains, subfamily A, | −4.763975 | 313 |
| | member 8A | | |
| MSMB | microseminoprotein, beta- | −54.942 | 314 |
| NCOA4 | Ncoa4: nuclear receptor coactivator 4 | −4.371086 | 315 |
| NKX3-1 | NK3 homeobox 1 | −5.818 | 316 |
| NLRP10 | NLR family, pyrin domain containing 10 | −3.205 | 317 |
| NNAT | Nnat: neuronatin | −5.353293 | 318 |
| ONECUT2 | one cut homeobox 2 | −16.394 | 319 |
| PADI1 | Padi1: peptidyl arginine deiminase, type I | −3.112583 | 320 |
| PAPSS2 | Papss2: 3′-phosphoadenosine 5′-phosphosulfate | −3.043293 | 321 |
| | synthase 2 | | |
| PDK2 | Pdk2: pyruvate dehydrogenase kinase, isoenzyme 2 | −2.090604 | 322 |
| PEX1 | peroxisomal biogenesis factor 1 | −2.268 | 323 |
| PFN2 | Pfn2: profilin 2 | −2.213251 | 324 |
| PINK1 | Pink1: PTEN induced putative kinase 1 | −2.017223 | 325 |
| PITX2 | paired-like homeodomain 2 | −4.344 | 326 |
| PLLP | Pllp: plasma membrane proteolipid | −3.416169 | 327 |
| PM20D1 | peptidase M20 domain containing 1 | −6.322 | 328 |
| PPARG | Pparg: peroxisome proliferator activated receptor gamma | −3.063091 | 329 |
| PPFIBP2 | PTPRF interacting protein, binding protein 2 (liprin | −2.063 | 330 |
| | beta 2) | | |
| PRLR | prolactin receptor | −5.992 | 331 |
| PSCA | Psca: prostate stem cell antigen | −44.76312 | 332 |
| PTEN | phosphatase and tensin homolog | Knockout | 333 |
| PTGS1 | Ptgs1: prostaglandin-endoperoxide synthase 1 | −2.729186 | 334 |
| PTPRZ1 | protein tyrosine phosphatase, receptor-type, Z | −5.826 | 335 |
| | polypeptide 1 | | |
| RAB17 | Rab17: RAB17, member RAS oncogene family | −2.637571 | 336 |
| RAB27B | Rab27b: RAB27b, member RAS oncogene family | −2.252252 | 337 |
| REG3G | regenerating islet-derived 3 gamma | −12.093 | 338 |
| RNASE1 | ribonuclease, RNase A family, 1 (pancreatic) | −8.629 | 339 |
| RPESP | Gm106: gene model 106, (NCBI) | −2.493949 | 340 |
| RTN4RL1 | Rtn4rl1: reticulon 4 receptor-like 1 | −2.303763 | 341 |
| SATB1 | SATB homeobox 1 | −2.993 | 342 |
| SCNN1A | Scnn1a: sodium channel, nonvoltage-gated, type 1, alpha | −3.184111 | 343 |
| SEMA4G | sema domain, immunoglobulin domain (Ig), | −2.695 | 344 |
| | transmembrane domain (TM) and short cytoplasmic | | |
| | domain, (semaphorin) 4G | | |
| SLC12A7 | Slc12a7: solute carrier family 12, member 7 | −2.507681 | 345 |
| SLC16A7 | solute carrier family 16, member 7 (monocarboxylic | −7.11 | 346 |
| | acid transporter 2) | | |
| SLC25A26 | solute carrier family 25, member 26 | −5.572 | 347 |
| SMAD4 | SMAD family member 4 | Knockout | 348 |
| SORD | Sord: sorbitol dehydrogenase | −2.372807 | 349 |
| SPINT1 | serine peptidase inhibitor, Kunitz type 1 | −2.05 | 350 |
| SPRR2G | Sprr2a: small proline-rich protein 2A | −3.415109 | 351 |
| STARD10 | Stard10: START domain containing 10 | −2.280847 | 352 |
| STAT5A | Stat5a: signal transducer and activator of transcription 5A | −2.794118 | 353 |
| SUOX | sulfite oxidase | −3.275 | 354 |
| TBX3 | Tbx3: T-box 3 | −2.020364 | 355 |
| TESC | Tesc: tescalcin | −5.666667 | 356 |
| TFF3 | Tff3: trefoil factor 3, intestinal | −13.59246 | 357 |
| TGM4 | transglutaminase 4 (prostate) | −31.185 | 358 |
| TIMP4 | Timp4: tissue inhibitor of metalloproteinase 4 | −2.755187 | 359 |
| TMEM159 | Tmem159: transmembrane protein 159 | −2.956762 | 360 |
| TMEM45B | Tmem45b: transmembrane protein 45b | −9.007153 | 361 |
| TMEM56 | transmembrane protein 56 | −2.609 | 362 |
| TOX3 | TOX high mobility group box family member 3 | −2.982 | 363 |
| TRIM2 | Trim2: tripartite motif protein 2 | −2.312697 | 364 |
| TSPAN8 | Tspan8: tetraspanin 8 | −2.449973 | 365 |
| TTR | Ttr: transthyretin | −160.1633 | 366 |
| TYRO3 | TYRO3 protein tyrosine kinase | −2.026 | 367 |
| UGT2B15 | Ugt2b35: UDP glucuronosyltransferase 2 family, | −14.95495 | 368 |
| | polypeptide B35 | | |
| UPK1A | Upk1a: uroplakin 1A | −5.459103 | 369 |
| UPK1B | Upk1b: uroplakin 1B | −2.546784 | 370 |
| ZBTB16 | Zbtb16: zinc finger and BTB domain containing 16 | −3.264302 | 371 |
| ZDHHC14 | Zdhhc14: zinc finger, DHHC domain containing 14 | −2.030303 | 372 |

One skilled in the art will recognize that the PCDETERMINANTS presented herein encompasses all forms and variants, including but not limited to, polymorphisms, isoforms, mutants, derivatives, precursors including nucleic acids and pro-proteins, cleavage products, receptors (including soluble and transmembrane receptors), ligands, protein-ligand complexes, and post-translationally modified variants (such as cross-linking or glycosylation), fragments, and degradation products, as well as any multi-unit nucleic acid, protein, and glycoprotein structures comprised of any of the PCDETERMINANTS as constituent sub-units of the fully assembled structure.

One skilled in the art will note that the above listed PCDETERMINANTS come from a diverse set of physiological and biological pathways, including many which are not commonly accepted to be related to metastatic disease. These groupings of different PCDETERMINANTS, even within those high significance segments, may presage differing signals of the stage or rate of the progression of the disease. Such distinct groupings of PCDETERMINANTS may allow a more biologically detailed and clinically useful signal from the PCDETERMINANTS as well as opportunities for pattern recognition within the PCDETERMINANT algorithms combining the multiple PCDETERMINANT signals.

The present invention concerns, in one aspect, a subset of PCDETERMINANTS; other PCDETERMINANTS and even biomarkers which are not listed in the above Table 1, but related to these physiological and biological pathways, may prove to be useful given the signal and information provided from these studies. To the extent that other biomarker pathway participants (i.e., other biomarker participants in common pathways with those biomarkers contained within the list of PCDETERMINANTS in the above Table 1) are also relevant pathway participants in cancer or a metastatic event, they may be functional equivalents to the biomarkers thus far disclosed in Table 1. These other pathway participants are also considered PCDETERMINANTS in the context of the present invention, provided they additionally share certain defined characteristics of a good biomarker, which would include both involvement in the herein disclosed biological processes and also analytically important characteristics such as the bioavailability of said biomarkers at a useful signal to noise ratio, and in a useful and accessible sample matrix such as blood serum or a tumor biopsy. Such requirements typically limit the diagnostic usefulness of many members of a biological pathway, and frequently occurs only in pathway members that constitute secretory substances, those accessible on the plasma membranes of cells, as well as those that are released into the serum upon cell death, due to apoptosis or for other reasons such as endothelial remodeling or other cell turnover or cell necrotic processes, whether or not they are related to the disease progression of cancer or metastatic event. However, the remaining and future biomarkers that meet this high standard for PCDETERMINANTS are likely to be quite valuable.

Furthermore, other unlisted biomarkers will be very highly correlated with the biomarkers listed as PCDETERMINANTS in Table 1 (for the purpose of this application, any two variables will be considered to be “very highly correlated” when they have a Coefficient of Determination (R²) of 0.5 or greater). The present invention encompasses such functional and statistical equivalents to the aforementioned PCDETERMINANTS. Furthermore, the statistical utility of such additional PCDETERMINANTS is substantially dependent on the cross-correlation between multiple biomarkers and any new biomarkers will often be required to operate within a panel in order to elaborate the meaning of the underlying biology.

One or more, preferably two or more of the listed PCDETERMINANTS can be detected in the practice of the present invention. For example, two (2), three (3), four (4), five (5), ten (10), fifteen (15), twenty (20), forty (40), fifty (50), seventy-five (75), one hundred (100), one hundred and twenty five (125), one hundred and fifty (150), one hundred and seventy-five (175), two hundred (200), two hundred and ten (210), two hundred and twenty (220), two hundred and thirty (230), two hundred and forty (240), two hundred and fifty (250), two hundred and sixty (260) or more, two hundred and seventy (270) or more, two hundred and eighty (280) or more, two hundred and ninety (290) or more, three hundred (300) or more, three hundred and ten (310) or more, three hundred and twenty (320) or more, three hundred and thirty (330) or more, three hundred and forty (340) or more, three hundred and fifty (350) or more, three hundred and sixty (360) or more, three hundred and seventy (370) or more PCDETERMINANTS can be detected.

In some aspects, all 372 PCDETERMINANTS listed herein can be detected. Preferred ranges from which the number of PCDETERMINANTS can be detected include ranges bounded by any minimum selected from between one and 372, particularly two, four, five, ten, twenty, fifty, seventy-five, one hundred, one hundred and twenty five, one hundred and fifty, one hundred and seventy-five, two hundred, two hundred and ten, two hundred and twenty, two hundred and thirty, two hundred and forty, two hundred and fifty, paired with any maximum up to the total known PCDETERMINANTS, particularly four, five, ten, twenty, fifty, and seventy-five. Particularly preferred ranges include two to five (2-5), two to ten (2-10), two to fifty (2-50), two to seventy-five (2-75), two to one hundred (2-100), five to ten (5-10), five to twenty (5-20), five to fifty (5-50), five to seventy-five (5-75), five to one hundred (5-100), ten to twenty (10-20), ten to fifty (10-50), ten to seventy-five (10-75), ten to one hundred (10-100), twenty to fifty (20-50), twenty to seventy-five (20-75), twenty to one hundred (20-100), fifty to seventy-five (50-75), fifty to one hundred (50-100), one hundred to one hundred and twenty-five (100-125), one hundred and twenty-five to one hundred and fifty (125-150), one hundred and fifty to one hundred and seventy five (150-175), one hundred and seventy-five to two hundred (175-200), two hundred to two hundred and ten (200-210), two hundred and ten to two hundred and twenty (210-220), two hundred and twenty to two hundred and thirty (220-230), two hundred and thirty to two hundred and forty (230-240), two hundred and forty to two hundred and fifty (240-250), two hundred and fifty to two hundred and sixty (250-260).

Construction of PCDETERMINANT Panels

Groupings of PCDETERMINANTS can be included in “panels.” A “panel” within the context of the present invention means a group of biomarkers (whether they are PCDETERMINANTS, clinical parameters, or traditional laboratory risk factors) that includes more than one PCDETERMINANT. A panel can also comprise additional biomarkers, e.g., clinical parameters, traditional laboratory risk factors, known to be present or associated with cancer or cancer metastasis, in combination with a selected group of the PCDETERMINANTS listed in Table 1.

As noted above, many of the individual PCDETERMINANTS, clinical parameters, and traditional laboratory risk factors listed, when used alone and not as a member of a multi-biomarker panel of PCDETERMINANTS, have little or no clinical use in reliably distinguishing individual normal subjects, subjects at risk for having a metastatic event, and subjects having cancer from each other in a selected general population, and thus cannot reliably be used alone in classifying any subject between those three states. Even where there are statistically significant differences in their mean measurements in each of these populations, as commonly occurs in studies which are sufficiently powered, such biomarkers may remain limited in their applicability to an individual subject, and contribute little to diagnostic or prognostic predictions for that subject. A common measure of statistical significance is the p-value, which indicates the probability that an observation has arisen by chance alone; preferably, such p-values are 0.05 or less, representing a 5% or less chance that the observation of interest arose by chance. Such p-values depend significantly on the power of the study performed.

Despite this individual PCDETERMINANT performance, and the general performance of formulas combining only the traditional clinical parameters and few traditional laboratory risk factors, the present inventors have noted that certain specific combinations of two or more PCDETERMINANTS can also be used as multi-biomarker panels comprising combinations of PCDETERMINANTS that are known to be involved in one or more physiological or biological pathways, and that such information can be combined and made clinically useful through the use of various formulae, including statistical classification algorithms and others, combining and in many cases extending the performance characteristics of the combination beyond that of the individual PCDETERMINANTS. These specific combinations show an acceptable level of diagnostic accuracy, and, when sufficient information from multiple PCDETERMINANTS is combined in a trained formula, often reliably achieve a high level of diagnostic accuracy transportable from one population to another.

The general concept of how two less specific or lower performing PCDETERMINANTS are combined into novel and more useful combinations for the intended indications, is a key aspect of the invention. Multiple biomarkers can often yield better performance than the individual components when proper mathematical and clinical algorithms are used; this is often evident in both sensitivity and specificity, and results in a greater AUC. Secondly, there is often novel unperceived information in the existing biomarkers, as such was necessary in order to achieve through the new formula an improved level of sensitivity or specificity. This hidden information may hold true even for biomarkers which are generally regarded to have suboptimal clinical performance on their own. In fact, the suboptimal performance in terms of high false positive rates on a single biomarker measured alone may very well be an indicator that some important additional information is contained within the biomarker results—information which would not be elucidated absent the combination with a second biomarker and a mathematical formula.

Several statistical and modeling algorithms known in the art can be used to both assist in PCDETERMINANT selection choices and optimize the algorithms combining these choices. Statistical tools such as factor and cross-biomarker correlation/covariance analyses allow more rationale approaches to panel construction. Mathematical clustering and classification tree showing the Euclidean standardized distance between the PCDETERMINANTS can be advantageously used. Pathway informed seeding of such statistical classification techniques also may be employed, as may rational approaches based on the selection of individual PCDETERMINANTS based on their participation across in particular pathways or physiological functions.

Ultimately, formula such as statistical classification algorithms can be directly used to both select PCDETERMINANTS and to generate and train the optimal formula necessary to combine the results from multiple PCDETERMINANTS into a single index. Often, techniques such as forward (from zero potential explanatory parameters) and backwards selection (from all available potential explanatory parameters) are used, and information criteria, such as AIC or BIC, are used to quantify the tradeoff between the performance and diagnostic accuracy of the panel and the number of PCDETERMINANTS used. The position of the individual PCDETERMINANT on a forward or backwards selected panel can be closely related to its provision of incremental information content for the algorithm, so the order of contribution is highly dependent on the other constituent PCDETERMINANTS in the panel.

Construction of Clinical Algorithms

Any formula may be used to combine PCDETERMINANT results into indices useful in the practice of the invention. As indicated above, and without limitation, such indices may indicate, among the various other indications, the probability, likelihood, absolute or relative risk, time to or rate of conversion from one to another disease states, or make predictions of future biomarker measurements of metastatic disease. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from PCDETERMINANT results in the relevant training population. Amongst other uses, such formula may be intended to map the feature space derived from one or more PCDETERMINANT inputs to a set of subject classes (e.g. useful in predicting class membership of subjects as normal, at risk for having a metastatic event, having cancer), to derive an estimation of a probability function of risk using a Bayesian approach (e.g. the risk of cancer or a metastatic event), or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (RF, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual PCDETERMINANT measurement into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on Clinical Parameters such as age, gender, race, or sex, where specific formula are used solely on subjects within a class or continuously combining a Clinical Parameter as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al, (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al, 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula. An example of this is the presentation of absolute risk, and confidence intervals for that risk, derived using an actual clinical study, chosen with reference to the output of the recurrence score formula in the Oncotype Dx product of Genomic Health, Inc. (Redwood City, Calif.). A further modification is to adjust for smaller sub-populations of the study based on the output of the classifier or risk formula and defined and selected by their Clinical Parameters, such as age or sex.

Combination with Clinical Parameters and Traditional Laboratory Risk Factors

Any of the aforementioned Clinical Parameters may be used in the practice of the invention as a PCDETERMINANT input to a formula or as a pre-selection criteria defining a relevant population to be measured using a particular PCDETERMINANT panel and formula. As noted above, Clinical Parameters may also be useful in the biomarker normalization and pre-processing, or in PCDETERMINANT selection, panel construction, formula type selection and derivation, and formula result post-processing. A similar approach can be taken with the Traditional Laboratory Risk Factors, as either an input to a formula or as a pre-selection criterium.

Measurement of PCDETERMINANTS

The actual measurement of levels or amounts of the PCDETERMINANTS can be determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, amounts of PCDETERMINANTS can be measured using reverse-transcription-based PCR assays (RT-PCR), e.g., using primers specific for the differentially expressed sequence of genes or by branch-chain RNA amplification and detection methods by Panomics, Inc. Amounts of PCDETERMINANTS can also be determined at the protein level, e.g., by measuring the levels of peptides encoded by the gene products described herein, or subcellular localization or activities thereof using technological platform such as for example AQUA® (HistoRx, New Haven, Conn.) or U.S. Pat. No. 7,219,016. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed.

The PCDETERMINANT proteins, polypeptides, mutations, and polymorphisms thereof can be detected in any suitable manner, but is typically detected by contacting a sample from the subject with an antibody which binds the PCDETERMINANT protein, polypeptide, mutation, or polymorphism and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Immunoassays carried out in accordance with the present invention may be homogeneous assays or heterogeneous assays. In a homogeneous assay the immunological reaction usually involves the specific antibody (e.g., anti-PCDETERMINANT protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

Antibodies can also be useful for detecting post-translational modifications of PCDETERMINANT proteins, polypeptides, mutations, and polymorphisms, such as tyrosine phosphorylation, threonine phosphorylation, serine phosphorylation, glycosylation (e.g., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF) (Wirth, U. et al. (2002) Proteomics 2(10): 1445-51).

For PCDETERMINANT proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant K_{M} using known algorithms, such as the Hill plot, Michaelis-Menten equation, linear regression plots such as Lineweaver-Burk analysis, and Scatchard plot.

Using sequence information provided by the database entries for the PCDETERMINANT sequences, expression of the PCDETERMINANT sequences can be detected (if present) and measured using techniques well known to one of ordinary skill in the art. For example, sequences within the sequence database entries corresponding to PCDETERMINANT sequences, or within the sequences disclosed herein, can be used to construct probes for detecting PCDETERMINANT RNA sequences in, e.g., Northern blot hybridization analyses or methods which specifically, and, preferably, quantitatively amplify specific nucleic acid sequences. As another example, the sequences can be used to construct primers for specifically amplifying the PCDETERMINANT sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), e.g., using primers specific for the differentially expressed sequences. RNA can also be quantified using, for example, other target amplification methods (e.g., TMA, SDA, NASBA), or signal amplification methods (e.g., bDNA), and the like.

Alternatively, PCDETERMINANT protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. (See, WO 04/056456 and WO 04/088309, each of which are hereby incorporated by reference in their entireties) In this regard, other PCDETERMINANT analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others. Other PCDETERMINANT metabolites can be similarly detected using reagents that are specifically designed or tailored to detect such metabolites.

Kits

The invention also includes a PCDETERMINANT-detection reagent, e.g., nucleic acids that specifically identify one or more PCDETERMINANT nucleic acids by having homologous nucleic acid sequences, such as oligonucleotide sequences, complementary to a portion of the PCDETERMINANT nucleic acids or antibodies to proteins encoded by the PCDETERMINANT nucleic acids packaged together in the form of a kit. The oligonucleotides can be fragments of the PCDETERMINANT genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a Northern hybridization or a sandwich ELISA as known in the art.

For example, PCDETERMINANT detection reagents can be immobilized on a solid matrix such as a porous strip to form at least one PCDETERMINANT detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of PCDETERMINANTS present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by PCDETERMINANTS 1-372. In various embodiments, the expression of 2, 3, 4, 5, 6, 7, 8, 9, 10, 15, 20, 25, 40, 50, 100, 125, 150, 175, 200, 250, 275 or more of the sequences represented by PCDETERMINANTS 1-372 can be identified by virtue of binding to the array. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.).

Suitable sources for antibodies for the detection of PCDETERMINANTS include commercially available sources such as, for example, Abazyme, Abnova, Affinity Biologicals, AntibodyShop, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, nucleic acid probes, e.g., oligonucleotides, aptamers, siRNAs, antisense oligonucleotides, against any of the PCDETERMINANTS in Table 1.

Methods of Treating or Preventing Cancer

The invention provides a method for treating, preventing or alleviating a symptom of cancer in a subject by decreasing expression or activity of PCDETERMINANTS 1-245 or increasing expression or activity of PCDETERMINANTS 246-272 Therapeutic compounds are administered prophylactically or therapeutically to subject suffering from at risk of (or susceptible to) developing cancer. Such subjects are identified using standard clinical methods or by detecting an aberrant level of expression or activity of (e.g., PCDETERMINANTS 1-372). Therapeutic agents include inhibitors of cell cycle regulation, cell proliferation, and protein kinase activity.

The therapeutic method includes increasing the expression, or function, or both of one or more gene products of genes whose expression is decreased (“underexpressed genes”) in a cancer cell relative to normal cells of the same tissue type from which the cancer cells are derived. In these methods, the subject is treated with an effective amount of a compound, which increases the amount of one of more of the underexpressed genes in the subject. Administration can be systemic or local. Therapeutic compounds include a polypeptide product of an underexpressed gene, or a biologically active fragment thereof a nucleic acid encoding an underexpressed gene and having expression control elements permitting expression in the cancer cells; for example an agent which increases the level of expression of such gene endogenous to the cancer cells (i.e., which up-regulates expression of the underexpressed gene or genes). Administration of such compounds counter the effects of aberrantly-under expressed of the gene or genes in the subject's cells and improves the clinical condition of the subject

The method also includes decreasing the expression, or function, or both, of one or more gene products of genes whose expression is aberrantly increased (“overexpressed gene”) in cancer cells relative to normal cells. Expression is inhibited in any of several ways known in the art. For example, expression is inhibited by administering to the subject a nucleic acid that inhibits, or antagonizes, the expression of the overexpressed gene or genes, e.g., an antisense oligonucleotide which disrupts expression of the overexpressed gene or genes.

Alternatively, function of one or more gene products of the overexpressed genes is inhibited by administering a compound that binds to or otherwise inhibits the function of the gene products. For example, the compound is an antibody which binds to the overexpressed gene product or gene products.

These modulatory methods are performed ex vivo or in vitro (e.g., by culturing the cell with the agent) or, alternatively, in vivo (e.g., by administering the agent to a subject). The method involves administering a protein or combination of proteins or a nucleic acid molecule or combination of nucleic acid, molecules as therapy to counteract aberrant expression or activity of the differentially expressed genes.

Diseases and disorders that are characterized by increased (relative to a subject not suffering from the disease or disorder) levels or biological activity of the genes may be treated with therapeutics that antagonize (i.e., reduce or inhibit) activity of the overexpressed gene or genes. Therapeutics that antagonize activity are administered therapeutically or prophylactically. (e.g. vaccines)

Therapeutics that may be utilized include, e.g., (i) a polypeptide, or analogs, derivatives, fragments or homologs thereof of the overexpressed or underexpressed sequence or sequences; (ii) antibodies to the overexpressed or underexpressed sequence or sequences; (iii) nucleic acids encoding the over or underexpressed sequence or sequences; (iv) antisense nucleic acids or nucleic acids that are “dysfunctional” (i.e., due to a heterologous insertion within the coding sequences of coding sequences of one or more overexpressed or underexpressed sequences); or (v) modulators (i.e., inhibitors, agonists and antagonists that alter the interaction between an over/underexpressed polypeptide and its binding partner. The dysfunctional antisense molecule are utilized to “knockout” endogenous function of a polypeptide by homologous recombination (see, e.g., Capecchi, Science 244: 1288-1292 1989)

Diseases and disorders that are characterized by decreased (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, a polypeptide (or analogs, derivatives, fragments or homologs thereof) or an agonist that increases bioavailability.

Generation of Transgenic Animals

Transgenic animals of the invention have one or both endogenous alleles of the Pten and Smad4 genes in nonfunctional form. Inactivation can be achieved by modification of the endogenous gene, usually, a deletion, substitution or addition to a coding region of the gene. The modification can prevent synthesis of a gene product or can result in a gene product lacking functional activity. Typical modifications are the introduction of an exogenous segment, such as a selection marker, within an exon thereby disrupting the exon or the deletion of an exon.

Inactivation of endogenous genes in mice can be achieved by homologous recombination between an endogenous gene in a mouse embryonic stem (ES) cell and a targeting construct. Typically, the targeting construct contains a positive selection marker flanked by segments of the gene to be targeted. Usually the segments are from the same species as the gene to be targeted (e.g., mouse). However, the segments can be obtained from another species, such as human, provided they have sufficient sequence identity with the gene to be targeted to undergo homologous recombination with it. Typically, the construct also contains a negative selection marker positioned outside one or both of the segments designed to undergo homologous recombination with the endogenous gene (see U.S. Pat. No. 6,204,061). Optionally, the construct also contains a pair of site-specific recombination sites, such as frt, position within or at the ends of the segments designed to undergo homologous recombination with the endogenous gene. The construct is introduced into ES cells, usually by electroporation, and undergoes homologous recombination with the endogenous gene introducing the positive selection marker and parts of the flanking segments (and frt sites, if present) into the endogenous gene. ES cells having undergone the desired recombination can be selected by positive and negative selection. Positive selection selects for cells that have undergone the desired homologous recombination, and negative selection selects against cells that have undergone negative recombination. These cells are obtained from preimplantation embryos cultured in vitro. Bradley et al., Nature 309, 255 258 (1984) (incorporated by reference in its entirety for all purposes). Transformed ES cells are combined with blastocysts from a non-human animal. The ES cells colonize the embryo and in some embryos form or contribute to the germline of the resulting chimeric animal. See Jaenisch, Science, 240, 1468 1474 (1988) (incorporated by reference in its entirety for all purposes). Chimeric animals can be bred with nontransgenic animals to generate heterozygous transgenic animals. Heterozygous animals can be bred with each other to generate homozygous animals. Either heterozygous or homozygous animals can be bred with a transgenic animal expressing the flp recombinase. Expression of the recombinase results in excision of the portion of DNA between introduced frt sites, if present.

Functional inactivation can also be achieved for other species, such as rats, rabbits and other rodents, bovines such as sheep, caprines such as goats, porcines such as pigs, and bovines such as cattle and buffalo, are suitable. For animals other than mice, nuclear transfer technology is preferred for generating functionally inactivated genes. See Lai et al., Sciences 295, 1089 92 (2002). Various types of cells can be employed as donors for nuclei to be transferred into oocytes, including ES cells and fetal fibrocytes. Donor nuclei are obtained from cells cultured in vitro into which a construct has been introduced and undergone homologous recombination with an endogenous gene, as described above (see WO 98/37183 and WO 98/39416, each incorporated by reference in their entirety for all purposes). Donor nuclei are introduced into oocytes by means of fusion, induced electrically or chemically (see any one of WO 97/07669, WO 98/30683 and WO 98/39416), or by microinjection (see WO 99/37143, incorporated by reference in its entirety for all purposes). Transplanted oocytes are subsequently cultured to develop into embryos which are subsequently implanted in the oviducts of pseudopregnant female animals, resulting in birth of transgenic offspring (see any one of WO 97/07669, WO 98/30683 and WO 98/39416). Transgenic animals bearing heterozygous transgenes can be bred with each other to generate transgenic animals bearing homozygous transgenes.

Some transgenic animals of the invention have both an inactivation of one or both alleles of Pten and Smad4 genes and a second transgene that confers an additional phenotype related to prostate cancer, its pathology or underlying biochemical processes. This disruption can be achievement by recombinase-mediated excision of Pten or Smad genes with embedded LoxP site (i.e., the current strain) or by for example mutational knock-in, and RNAi-mediated extinction of these genes either in a germline configuration or in somatic transduction of prostate epithelium in situ or in cell culture followed by reintroduction of these primary cells into the renal capsule or orthotopically. Other engineering strategies are also obvious including chimera formation using targeted ES clones that avoid germline transmission.

### EXAMPLES

### Example 1

### General Method

Pten and Smad4 Conditional Alleles, Genotyping and Expression Analysis

The Pten^{loxP} and Smad4^{loxP} conditional knockout alleles have been described elsewhere. Prostate epithelium-specific deletion was effected by the PB-Cre4²⁵. The PCR genotyping strategy for (i) Pten utilizes primers 1 (5′-CTTCGGAGCATGTCTGGCAATGC-3′; SEQ ID NO: 1), 2 (5′-CTGCACGAGACTAGTGAGACGTGC-3′; SEQ ID NO: 2), and 3 (5′-AAGGAAGAGGGTGGGGATAC-3′; SEQ ID NO: 3) and (ii) Smad4 utilizes primers 1 (5′-GGGAACAGAGCACAGGCCTCTGTGACAG-3′; SEQ ID NO: 4) and 2 (5′-TTCACTGTGTAGCCCCGCCTGTCCTGGA-3′; SEQ ID NO: 5). To detect the Smad4 deleted allele, primers 2 and 3 (5′-TGCTCTGAGCTCACAATTCTCCT-3′; SEQ ID NO: 6) were used.

For Western blot, analysis, tissues and cells were lysed with RIPA buffer (20 mM Tris pH 7.5, 150 mM NaCl, 1% Nonidet P-40, 0.5% Sodium Deoxycholate, 1 mM EDTA, 0.1% SDS) containing complete mini protease inhibitors (Roche) and phosphotase inhibitor. Western blots were obtained utilizing 20-50 μg of lysate protein, and were incubated with the antibodies against Smad4, p53 (IC12), pSmad2/3, pSmad1/5/8. (Cell Signaling Technology), p21^{Cip1} (M−19) and PTEN (A2B1) (Santa Cruz Biotechnology).

Tissue Analysis.

Normal and tumor tissues were fixed in 10% neutral-buffered formalin (Sigma) overnight, washed once with 1×PBS, transferred into 70% ethanol, and stored at 4° C. Tissues were processed by ethanol dehydration and embedded in paraffin by Histoserv Inc. (Gaithersburg, Md.) according to standard protocols. Sections (5 μm) were prepared for antibody detection and hematoxylin and eosin (H&E) staining. For immuno-histochemical studies, formalin-fixed paraffin-embedded sections were incubated overnight with rabbit polyclonal anti-PTEN or anti-p53 antibodies, followed by incubation with HRP-conjugated goat anti-rabbit IgG (H+L) secondary antibody (Vector), and visualized by incubating sections with DAB (Vector) and counterstained with hematoxylin and eosin. For immunofluorescence studies, prostate tumor cells were seeded on Lab-Tee 8 well slides at 5,000 cells/well, fixed with methanol at −20° C. for 10 min, stained with anti-CK8 and CK18 antibodies (CM5, Vector Laboratories), and visually processed via Image J (v1.38). Statistical significance was determined by Student's t-test. To assay senescence in prostate tissue of the various genotypes, frozen 6 μm sections were stained for SA-β-Gal as described elsewhere.

Establishment of Primary and Tet-Inducible Cell Lines.

Prostate cancer tissue was dissected from Pten^{loxP/loxP}; Smad4^{loxp/loxP}; PB-Cre4⁺ mouse, minced, and digested with 0.5% type I collagenase (Invitrogen) as described previously. After filtering through a 40-μm mesh, the trapped fragments were plated in tissue culture dishes coated with type I collagen (BD Pharmingen). Cells with typical epithelial morphology were collected, and single cells were seeded into each well of a 96-well plate. Three independence cell lines (3132-1, -2, and -3) were established and maintained in DMEM plus 10% fetal bovine serum (FBS; Omega Scientific), 25 μg/mL bovine pituitary extract, 5 μg/mL bovine insulin, and 6 ng/mL recombinant human epidermal growth factor (Sigma-Aldrich). To establish the Smad4 inducible cell lines, the mouse Pten/Smad4 null prostate tumor cell lines were transduced with pTRE-Tight vector (Clontech) containing the human SMAD4 coding region and tet-on stable cell lines were established according to the manufacturer's protocol. SMAD4 induction was achieved with 1 μg/ml doxycycline (dox) and verified by Western blot analysis.

Cell Culture-Based Assays.

For cell viability assays, prostate epithelial cells were plated in 96-well plates at 5000 cells/well in 100 μl of 5% charcoal-stripped FBS-containing medium. After 2 days incubation, the medium was replaced. Cells viability was measured on day 4 using CellTiter-Glo Luminescent Cell Viability Kit (Promega, Madison, Wis.) according to the manufacturer's protocol.

Transcriptomic, Genomic and In Silico Promoter Analyses.

For transcriptomic analyses, localized primary Pten^{pc-/-} and Pten^{pc-/-} Smad4^{pc-/-} mouse prostate tumors of comparable size and stage were isolated and total mRNA extracted, labeled and hybridized to Affymetrix GeneChip® Mouse Genome 430 2.0 Arrays by the Dana-Farber Cancer Institute Microarray Core Facility according to the manufacturer's protocol. Affymetrix mouse MOE430 raw data (CEL files) were pre-processed using robust multi-array analysis (RMA) of the affy package of Bioconductor The background-corrected and normalized intensity data were then analyzed using significance analysis of microarrays (SAM) to identify differentially expressed genes. Using a two-fold cut-off, we generated a supervised gene list that distinguishes Pten^{pc-/-} Smad4^{pc-/-} versus Pten^{pc-/-} samples. Intersection of the murine list with the human gene list produced a Pten/Smad4 orthologous set of 284 (200 up-regulated and 84 down-regulated) genes.

For in silico promoter analyses, the positional frequency matrices (PFM) for vertebrate-conserved binding sites were extracted from TRANSFAC Professional. The positional weight matrices (PWM) were constructed from PFM using the TFBS module. The TFBS module was also used to scan for binding sites within the 3-kb promoter sequences, which were downloaded from Ensembl via Biomart. The observed transcription factor binding sites in the target gene set were compared to those in a randomly selected background (mouse genome) gene set. A z-score and p-value (Statistics::Distributions from CPAN) were calculated to determine if a given binding site was over-represented in the target gene set.

To determine whether murine Pten/Smad4 are targeted for copy number alterations in human prostate cancer, we used resident genes in minimum common regions (MCRs) of metastatic human prostate cancer ACGH profiles, GSE8026 that were processed by circular binary segmentation as described previously. Common orthologous genes showing significant differential expression between Pten^{pc-/-} and Pten^{pc-/-} Smad4^{pc-/-} mouse prostate tumors as well as copy number alteration in metastatic human prostate tumors were selected for further computational analysis of clinically-annotated samples.

The Ingenuity Pathways Analysis program (http://www.ingenuity.com/index.html) was used to further analyze the cellular functions and pathways that were significantly regulated in the Pten^{pc-/-} and Pten^{pc-/-} Smad4^{pc-/-} PCA models.

Clinical Outcomes Analysis.

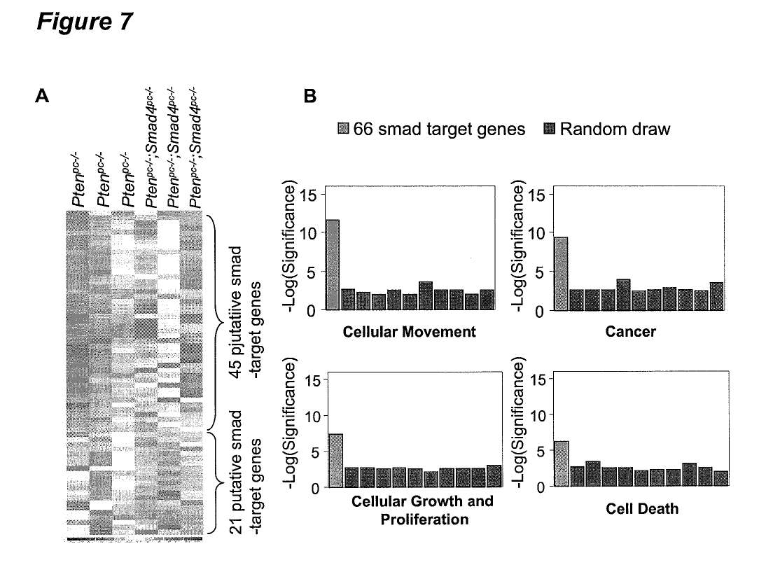

We implemented a “cross-species expression module comparison” approach (FIG. 7A) using 66 Smad target gene list emerging from the murine Pten/Smad4 transcriptome signature or its intersection with the metastatic human prostate ACGH dataset²⁷. Prostate cancer and breast cancer expression profiles were used to evaluate the prognostic value of these gene sets. The Spearman's rank correlation was used to identify two main clusters of clinically localized prostate cancer samples based on the 66-gene and 17-gene mRNA expression. To demonstrate statistic significance, we also selected 10 groups of random sets of 17 genes from the Glinsky prostate cancer or the Chang breast cancer profiling studies (refs).

Statistical Analysis.

Invasiveness-free and cumulative survival curves were obtained with Kaplan-Meier analysis as described previously. Statistical analyses were done by using GraphPad Prism 4 (GraphPadSoftware, San Diego, Calif.). Tumor incidence was plotted by using the Kaplan-Meier analysis. Statistical significance was measured by using the log-rank test.

### Example 2

### Pten Null Prostate Tumors Exhibit Marked TGF_{B}-Smad4 Pathway Activation

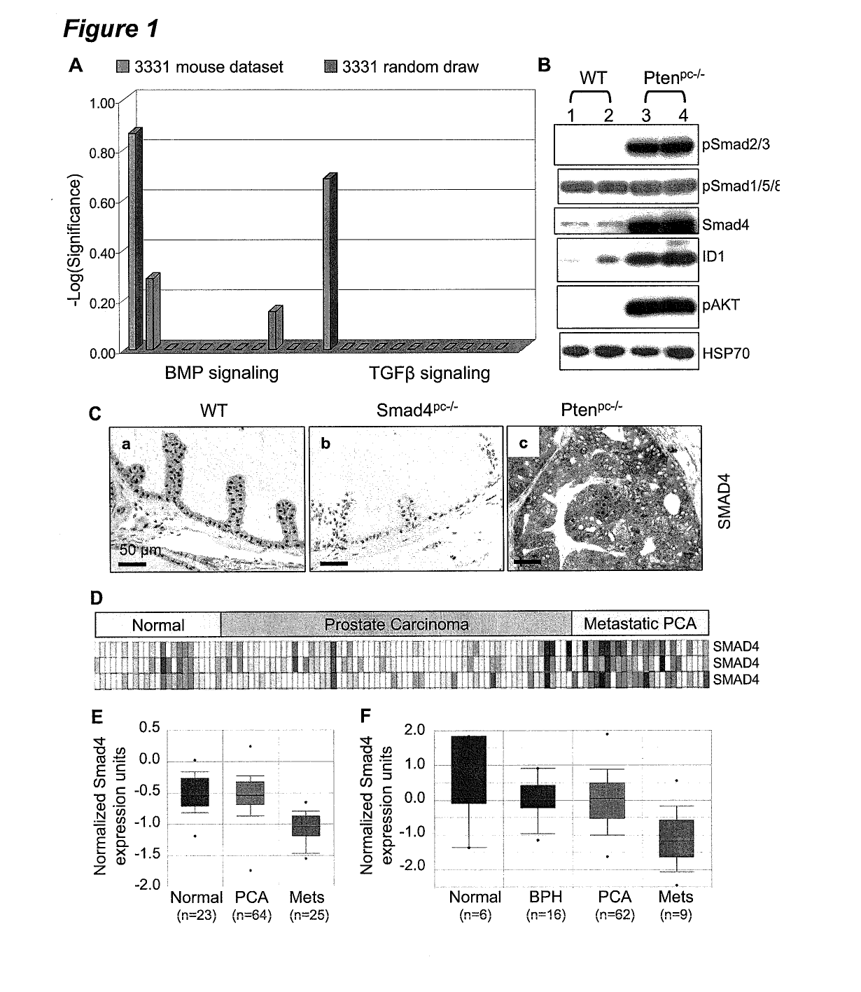

Prostate-specific deletion of the Pten tumor suppressor results in prostate intraepithelial neoplasia (PIN) and, following a long latency, occasional lesions can progress to adenocarcinoma, albeit with minimally invasive and metastatic features. To define checkpoints activated in Pten deficient PIN that might constrain progression to invasive and metastatic adenocarcinoma, we conducted an unbiased search using knowledge-based pathway analysis of differentially expressed genes in the anterior prostate high grade PIN disease arising in Pten^{loxP/loxP} Pb-Cre4 tumors versus anterior prostate epithelium from Pb-Cre4 mice at 15 weeks of age. This pathway analysis revealed hepatic steatosis, BMP and TGFβ as the top three networks enriched above that observed with randomly generated gene lists (FIG. 1A).

TGFβ superfamily of ligands, comprising of TGFβ, bone morphogenetic proteins (BMPs), and activins families, bind to a type II receptor, which recruits and phosphorylates a type I receptor. The type I receptor in turn phosphorylates receptor-regulated SMADs (R-SMADs). Upon activation of Smad2/3 by TGFb and Smad1/5/8 by BMPs, these receptor-activated R-Smads bind to common co-mediator Smad4 to form functional protein complexes which migrate to the nucleus to regulate diverse cancer-relevant gene targets. The enrichment of both BMP and TGFβ signaling networks in the differentially expressed gene list prompted direct molecular vfalidation of their common co-mediator Smad4. To this end, Western blot and IHC assays documented marked up-regulation of Smad4 expression, phosphor-activated Smad2/3, and the Smad-responsive target, ID1, in the Pten−/− PIN disease relative to wildtype prostate tissue (FIGS. 1B and C). In comparison, constitutively expressed pSmad1/5/8 showed only marginally increases in Pten−/− tumors relative to wildtype prostate tissue (FIG. 1B). In other words, these indolent Pten−/− prostate tumors had marked activation of the BMP/TGFβ-Smad4 signaling pathway, suggesting possible involvement of Smad4 in blocking prostate cancer progression. This hypothesis is in line with the observation that Smad4 expression in human PCA is significantly downregulated during progression from primary to metastatic disease (FIG. 1D-F).

### Example 3

### SMAD4 Constrains Progression of Pten Deficient Prostate Tumors

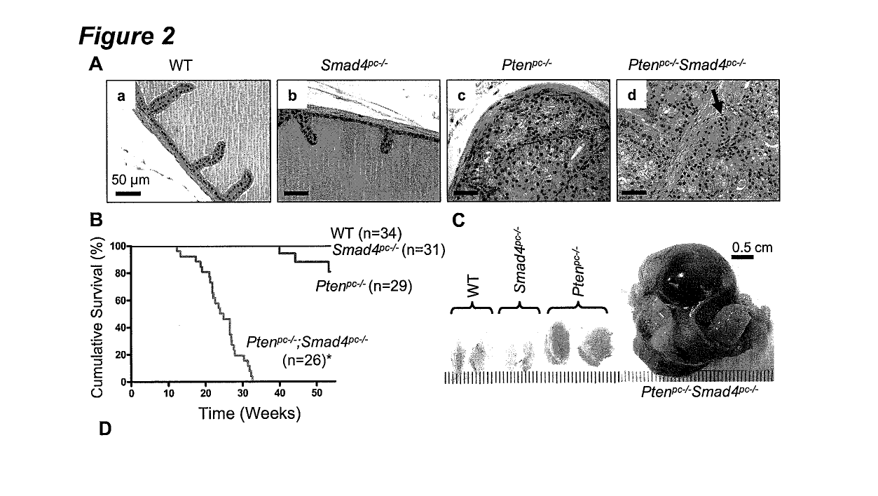

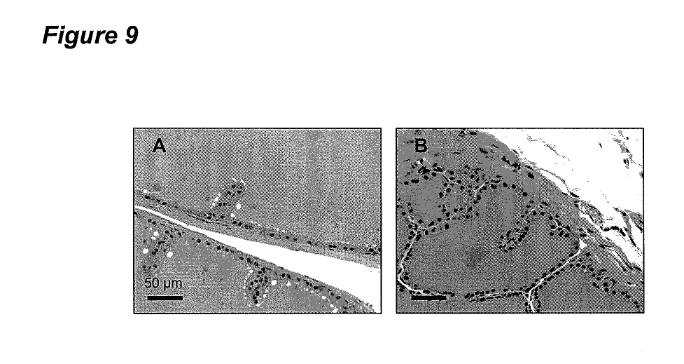

To genetically address this hypothetical Smad4-dependent progression block and its consequent inactivation in advanced disease, we utilized the prostate-specific deletor, Pb-Cre4, to specifically delete Pten and/or Smad4 in the prostate epithelium. The Pten^{loxP/loxP} Pb-Cre4 and Smad4^{loxP/loxP} Pb-Cre4 mice (hereafter Pten^{pc-/-} and Smad4^{pc-/-}) showed robust Cre-mediated recombination only in the prostate, specifically the anterior prostate, ventral prostate and dorsolateral prostate lobes (data not shown) as reported previously^{18,20}. In line with previous Pten studies^{18,20}, the Pten^{pc-/-} mice consistently developed high-grade PIN in all three lobes as early as 9 weeks of age, in contrast, PB-Cre4 (hereafter WT) and Smad4^{pc-/-} littermates exhibited normal prostate histology (FIG. 2A). Notably, through 2 years of age (FIGS. 9A and B), Smad4 deficiency had no discernable impact on prostate histology which remained tumor-free (n=15; data not shown).

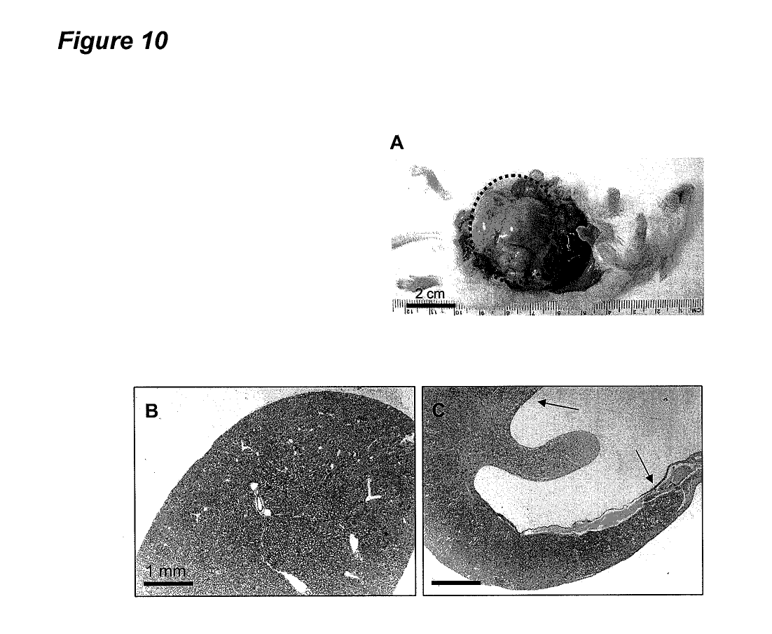

The Pten^{pc-/-} model shows a slowly progressive neoplastic phenotype with invasive features emerging after 17 to 24 weeks of age; most mice are alive at 1 year of age (FIG. 2B). In sharp contrast, Pten^{pc-/-} Smad4^{pc-/-} mice developed highly aggressive invasive PCA by 9 weeks of age (FIG. 2A,d), culminating in death by 32 weeks of age in all cases (FIG. 2B, C). These large prostate tumors produce bladder outlet obstruction and hydronephrosis—distention of the kidney due to outflow obstruction with consequent renal failure as a likely cause of mortality (FIG. 10).

To begin to understand the tumor biological basis for the Pten^{pc-/-} Smad4^{pc-/-} progression phenotype, we assessed the impact of Smad4 status on the levels of proliferation, apoptosis and senescence in the developing prostate tumors. We observed markedly increased proliferation in the Pten^{pc-/-} Smad4^{pc-/-} tumors, particularly along invasive tumor fronts; while the Pten^{pc-/-} tumors showed more modest proliferative activity (FIG. 3A, C). Also, consistent with these distinct proliferative profiles, we observe a marked decrease in SA-β-Gal activity in Pten^{pc-/-} Smad4^{pc-/-} tumors relative to Pten^{pc-/-} tumors (FIG. 3B, E), consistent with deactivation of the oncogene induced senescence (OIS) checkpoint. Finally, Pten^{pc-/-} Smad4^{pc-/-} and Pten^{pc-/-} tumors showed no differences in apoptotic cell death as measured by TUNEL assays (FIG. 3A, D).

### Example 4

### Loss of SMAD4 Drives a Fully Penetrant Invasive and Metastatic Phenotype

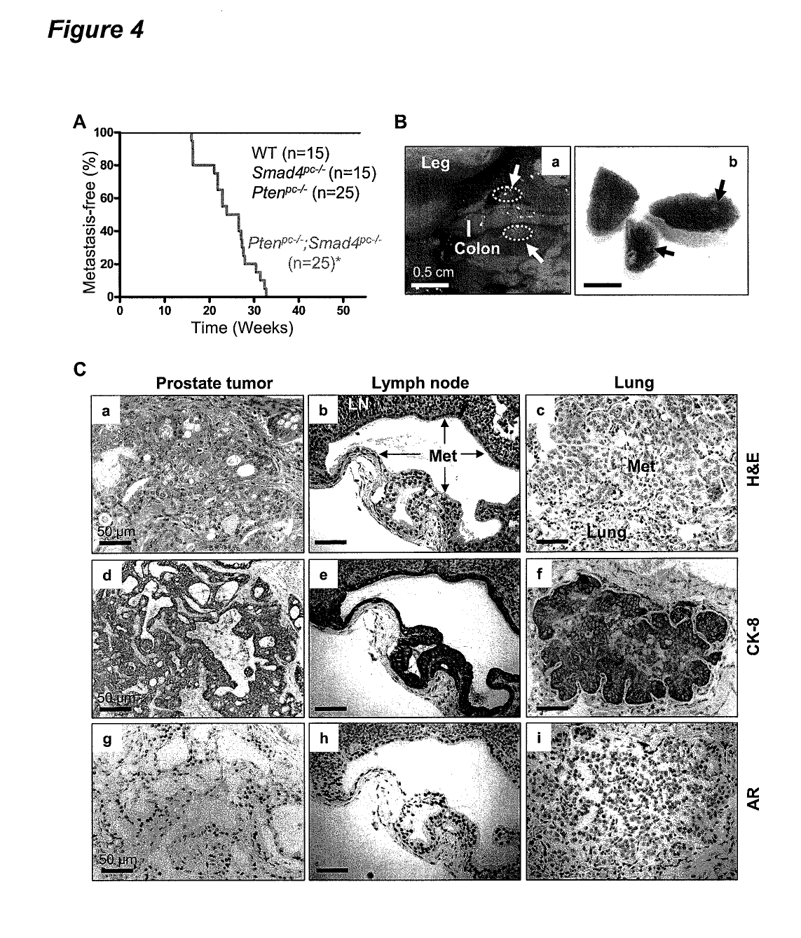

An obligate feature of lethal PCA in humans is progression to invasive and metastatic disease, prompting detailed serial and endpoint histopathological surveys of the Pten^{pc-/-} Smad4^{pc-/-} tumors. The Pten^{pc-/-} Smad4^{pc-/-} tumors showed penetration through the basement membrane as early as 9 weeks (n=7 examined); whereas during the same period, all Pten^{pc-/-} neoplasms (n=7 examined) were confined by the basement membrane (data not shown). Notably, in terminal endpoint surveys, all 25 tumor-bearing Pten^{pc-/-} Smad4^{pc-/-} mice showed metastatic spread to draining lymph nodes and 2 of these mice also possessed lung metastasis (FIG. 4A, 4B, a,b). The prostatic epithelial origin of the documented metastatic disease was confirmed by positive staining for cytokeratin (CK)8 and androgen receptor (AR) (FIG. 4C,e,f,). It is worth noting that none of the 25 Pten^{pc-/-} Smad4^{pc-/-} mice showed bone metastasis which may relate to rapid demise due to urinary obstruction and/or the need for genetic events beyond Smad4 loss to enable this key feature in human PCA (FIG. 10). In contrast, none of the 25 Pten^{pc-/-} tumor-bearing mice developed metastatic lesions up to 1 year of age (FIG. 4A), although 1 lumbar lymph node and 1 lung metastases were documented in 8 mice older than 1.5 years—an observation consistent with previous reports. To our knowledge, this is the first fully penetrant metastatic prostate adenocarcinoma model that, similar to human PCA, retains the prostate markers of CK and AR.

### Example 5

### Identification of PCDETERMINANTS and their Prognostic Utility in Human Prostate Cancer

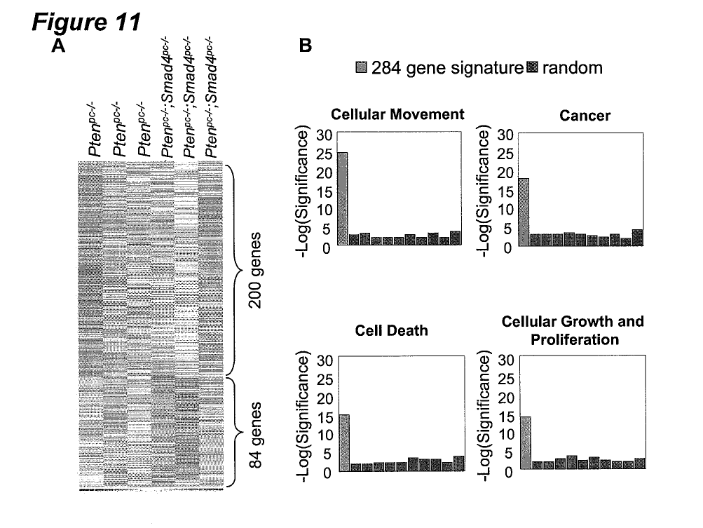

The strikingly different progression phenotypes of the Pten^{pc-/-} and Pten^{pc-/-} Smad4^{pc-/-} PCA models and the salient function of Smad4 as a sequence-specific transcription factor provided an ideal framework for comparative transcriptomic analysis to uncover how Smad4 might function to constrain malignant progression, specifically in prostate cancer. To that end, we obtained comparably sized early stage primary anterior lobe prostate tumors from both models at approximately 15 weeks of age—histological surveys documented the lack of metastatic disease in these mice (data not shown). Tumor samples were processed for histology, immunohistochemistry and RNA extraction for gene expression profiling. Initial comparative analysis with three tumors from each genotype identified 284 differentially expressed PCDETERMINANTS (Table 1A). Subsequent analysis with an expanded group of five tumors from each genotype identified an expanded group of 372 differentially expressed PCDETERMINANTS (Table 1B). Not surprisingly, unsupervised classification readily separated the Pten^{pc-/-}; Smad4^{pc-/-} and Pten^{pc-/-} tumors (data not shown). Considering the phenotypic difference between these two models, it was gratifying that knowledge-based pathway analysis of the 284 differentially expressed genes (200 up- and 84 down-regulated) pinpointed cell movement as the most significant functional category, followed by cancer, cell death, and cell growth and proliferation enriched in these pro-metastatic Pten^{pc-/-} Smad4^{pc-/-} primary tumors (FIG. 11).

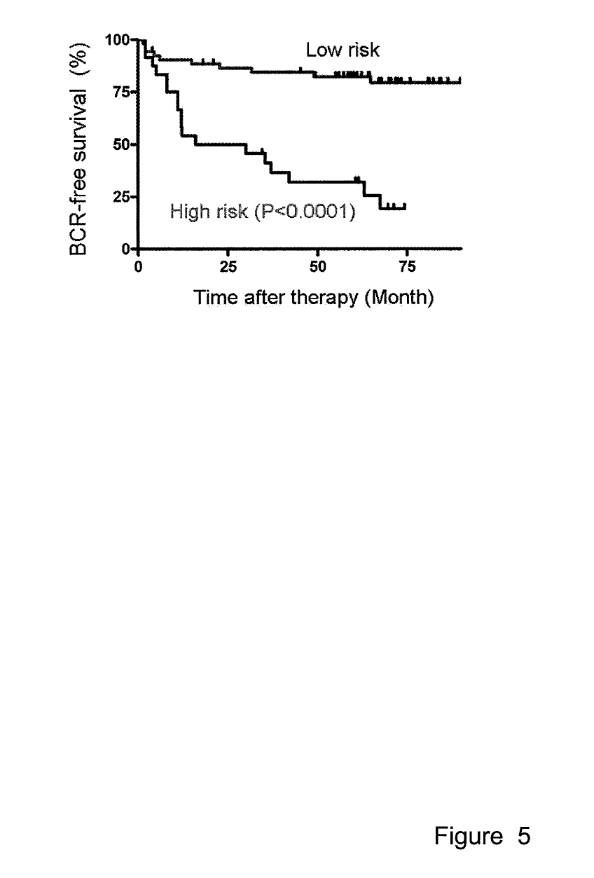

Next, we sought to confirm that PCDETERMINANTS discovered through comparison of murine prostate tumor expression profiles were relevant to human cancer, To this end, we utilized a human PCA gene expression dataset by Glinsky and colleagues¹, consisting of 79 clinically localized specimens annotated with time to PSA recurrence (so-called biochemical recurrence). Unsupervised classification by hierarchical clustering using the 284 PCDETERMINANTS listed on Table 1A stratified clinical patient samples into subgroups with significant clinical outcome for recurrence (FIG. 5, p<0.0001).

### Example 6

### Integrative Analyses Define a Set of Predicted SMAD4 Targets in Metastatic-Capable Primary Tumors

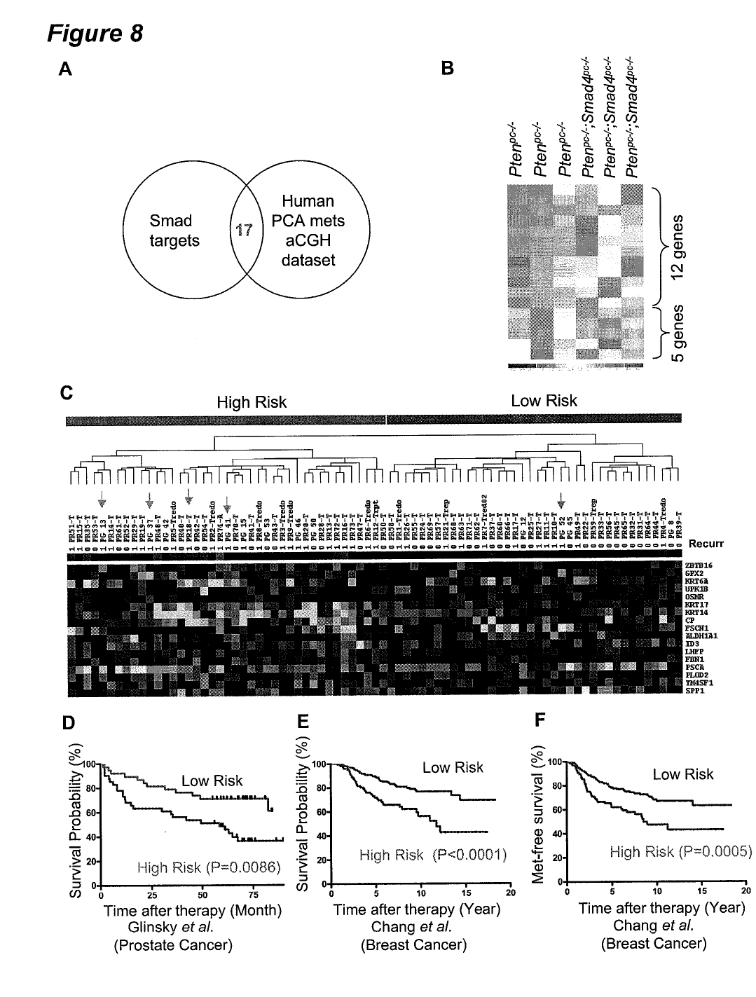

Next, we scanned the promoters of 284 PCDETERMINANTS for evolutionarily conserved Smad binding elements, identifying 66 predicted direct Smad4 transcriptional targets (FIG. 7A; see Table 2 for complete list). The knowledge-based pathway analysis of this 66-Smad4 transcriptional targets (45 up- and 21 down-regulated) pinpointed cell movement again as the most significant functional category (p=2.46×10⁻¹²), followed by cancer (p=3.77×10⁻¹⁰), cell growth and proliferation (p=4.14×10⁻⁸), and cell death (p=5.75×10⁻⁷) enriched in these pro-metastatic Pten^{pc-/-} Smad4^{pc-/-} primary tumors (FIG. 7B). Strikingly, 28 of 66 genes are functionally annotated as cell movement genes. This 66 gene list was further intersected with array-CGH profiles of human metastatic PCA¹⁹, reasoning that key Smad4-dependent progression driver events would themselves be targeted for genomic alterations in advanced disease, i.e., genes up-regulated upon loss of Smad4 would themselves be targeted for amplification, while down-regulated genes would be deleted. This cross-species yielded 17 genes (FIG. 8A) of which 5 have known links to cell movement (FSCN1, ID3, KRT6A, SPP1, and ZBTB16). Interestingly, comparative oncogenomics analyses in melanoma has recently identified FSCN1 as a key metastasis and prognosis PCdeterminant (data not shown), raising the possibility that our gene signature is relevant to invasion and metastatic processes and clinical outcomes across multiple tumor types.

### Example 7

### Cross-Species Triangulated Smad4 Transcriptional Targets are Linked to Clinical Outcome

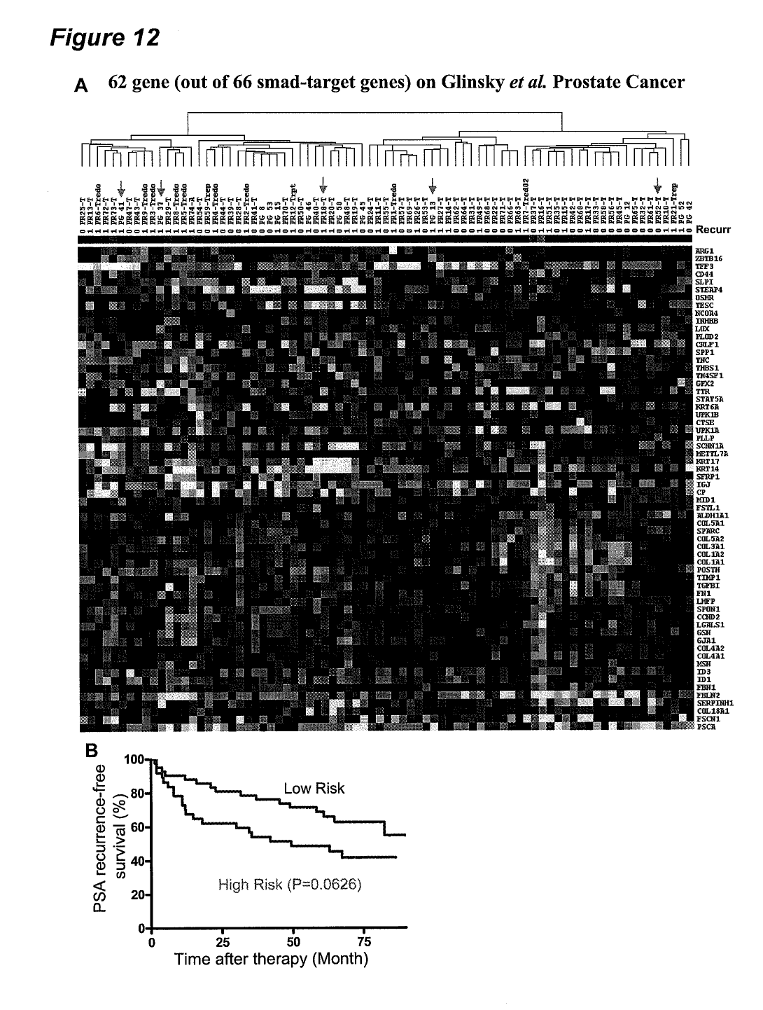

To garner evidence of human relevance for these evolutionarily-conserved predicted Smad4 targets and further credential this novel model of metastatic PCA, we assessed the ability of the 17 cross-species triangulated genes to stratify PSA-recurrence in human PCA relative to the murine-only 66 gene list. To this end, we utilized a human PCA gene expression dataset by Glinsky and colleagues¹⁵, consisting of 79 clinically localized specimens annotated with time to PSA recurrence (so-called biochemical recurrence). Unsupervised classification by hierarchical clustering using the 17-gene list assigned these patient tumors to one of two main branches (FIG. 7C). Albeit too small in sample size for statistical significance, 4 of 5 metastatic specimens in this cohort clustered in the high-risk group defined by these 17 genes (FIG. 7B). Moreover, Kaplan-Meier analysis of the two subclasses stratified by this 17-gene list showed significant differences in time-to-recurrence (p=0.0086) (FIG. 7D), while randomly selected lists (n=10) of 17 gene sets from the Glinsky profiling study¹⁵ failed to generate statistically significant separation (P=0.8610; 0.6086; 0.1827; 0.8338; 0.6391; 0.7918; 0.1814; 0.9851; 0.3946; 0.9201;). In comparison, the 66-gene set was not able to stratify patients into differential outcome subclasses (p=0.0626), substantiating that the cross-species filter has effectively culled noisy bystanders from the 66 genes list (FIG. 12).

Next, to assess whether the 17-gene list is specific to prostate, we performed similar analyses using outcome annotated expression data from 295 primary breast cancers²⁸. As shown in FIG. 8E, unsupervised clustering with the 17 genes subclassified these breast tumor samples into two groups with significant difference in overall survival (p<0.0001) and metastasis-free survival (p=0.0005; FIG. 8F). Randomly selected 17-gene lists (n=10) again failed to achieve any significant separation of the Kaplan-Meier curves (Supp info or fig). Whereas the 66-gene set was borderline performer in this task—overall survival (p=0.0263) and metastasis-free survival (p=0.0886).

Taken together, these correlative analyses demonstrating the power of these evolutionarily conserved Smad4 targets to classify human prostate and breast adenocarcinomas into good and poor outcome subclasses, along with the frequent and significant downregulation of Smad4 during progression (Oncomine data, show boxplots) in multiple human tumor types, serve to validate the Pten^{pc-/-} Smad4^{pc-/-} mouse as a highly relevant metastatic prostate model driven by signature events present in human PCA and support our integrative cross-species analytical approach.

### Example 8

### In Silico Analysis Reveals Cell Movement Genes are Differentially Expressed in Metastatic Pten/Smad4 Tumors Compared to Indolent PTEN Tumors

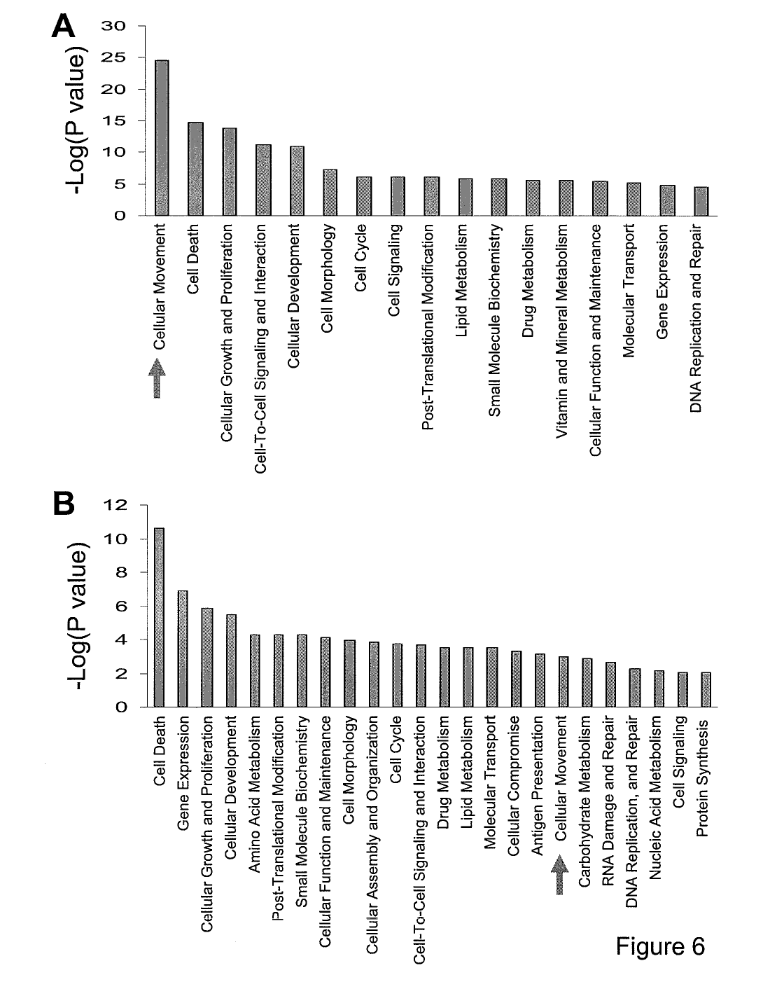

The strikingly different progression phenotypes of the Pten^{pc-/-} and Pten^{pc-/-} Smad4^{pc-/-} PCA models and the ability of the 284 gene panel to stratify human PCA patient populations underscore that the PCDETERMINANTS are functionally driving metastatic progression. To glean early insight into the types of biological activities conferred by these genes, we performed knowledge-based pathway analysis using Ingenuity Pathway Analysis (IPA) (Ingenuity Systems Inc., Redwood City, Calif.) (FIG. 6). Whereas the cell movement category ranked #18 in the invasive but not metastatic Pten^{pc-/-}; p53^{pc-/-} tumors (FIG. 6B), cell movement genes ranked #1 for the metastasis-prone Pten^{pc-/-}; Smad4^{pc-/-} tumors (FIG. 6A).

### Example 9

### PCDETERMINANTS Exhibit Progression Correlated Expression in Human Prostate Cancer

It is well established that genomic instability drives tumorigenesis, creating primary tumors comprised of heterogeneous subpopulations of cells with common and distinct genetic profiles. It thus stands to reason that, if a PCDETERMINANT-expressing sub-population within a primary tumor is endowed with a proliferative advantage and ultimately disseminates, the expression of the PCDETERMINANT would increase due to enriched representation in the more homogeneous derivative metastatic lesions. To assess for such progression-associated expression, the 372 PCDETERMINANTS were examined in the large compendium of prostate cancer expression profiling data on Oncomine. SEVENTY-FOUR (74) PCDETERMINANTS were found to exhibit progression-correlated expression in human prostate cancer (Table 4), further underscoring the relevance of PCDETERMINANTS to human cancer.

### Example 10

### Cross-Species and Cross-Platform Triangulated PCDETERMINANTS are Prognostic in Human Prostate Cancer

This metastasis signature comprising of 372 PCDETERMINANTS differentially expressed at the RNA level in metastatic-prone versus indolent mouse tumors was next interfaced with a large compendium of genes that reside in copy number aberrations (CNAs) in a human metastatic prostate cancer dataset¹⁹. We used resident genes in minimum common regions (MCRs) of metastatic human prostate cancer ACGH profiles, GSE8026¹⁹ that were processed by circular binary segmentation as described previously²⁴. Common orthologous genes showing significant differential expression between Pten^{pc-/-} and Pten^{pc-/-} Smad4^{pc-/-} mouse prostate tumors as well as copy number alteration in metastatic human prostate tumors were selected for further computational analysis. This analysis identified 56 PCDETERMINANTS (Table 7 which are differentially expressed at the RNA level in metastasis-prone mouse tumors and the DNA level in metastatic human prostate cancer (FIG. 6A).

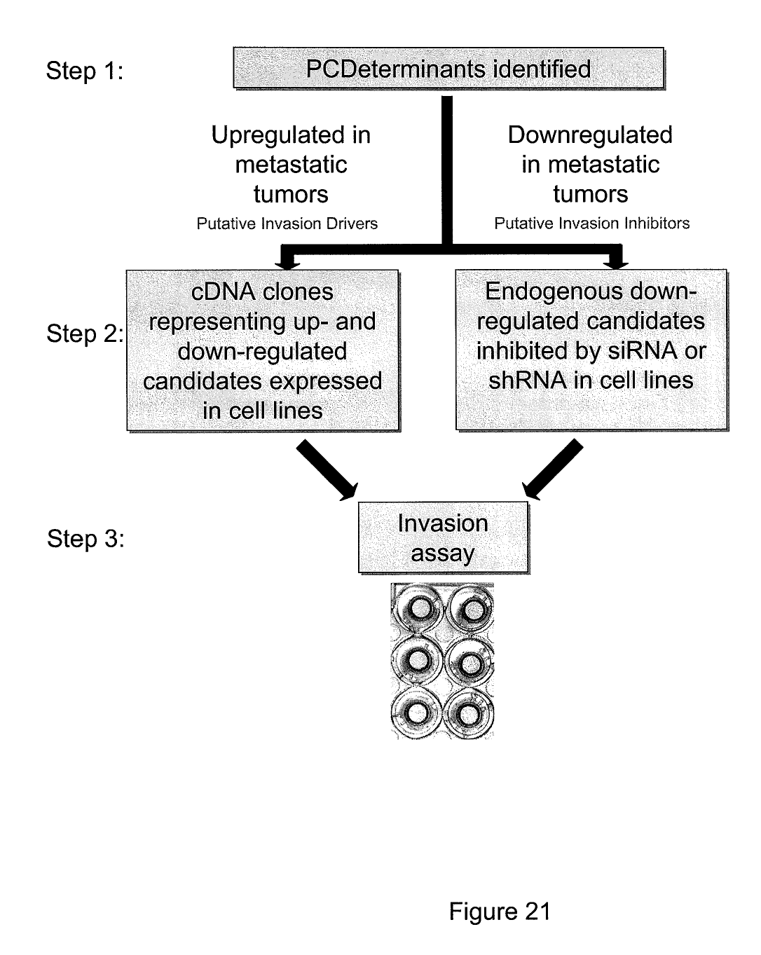

The 56 gene set (Table 7) was subsequently evaluated for prognostic utility on a prostate cancer gene expression data set. Patient samples were categorized into two major clusters (low risk group and high risk group) defined by the 56-gene signature. Kaplan-Meier analysis of biochemical recurrence (BCR) PSA level (>0.2 ng/ml) based on the groups defined by the 56-gene cluster. A statistically significant for BCR PSA recurrence-free survival (P=0.0018) compared with the “low-risk” cohort was found for the “high-risk” cohort (FIG. 21B).

### Example 11

### Genetic Screens to Identify PCDETERMINANTS Functionally Involved in Invasion

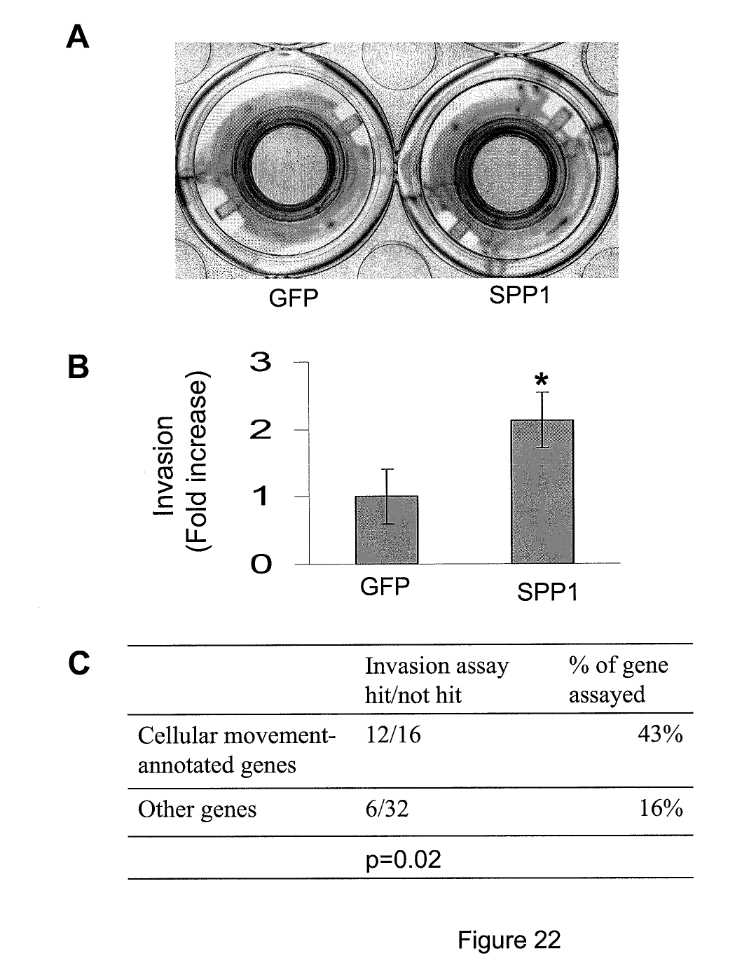

Genetic screens are useful to identify the subset of PCDETERMINANTS that functionally drive metastasis (FIG. 22). Heterologous overexpression of certain PCDETERMINANTS (in particular PCDETERMINANTS 1-245) increases invasive activity of human cells. Similarly, downregulation of certain PCDETERMINANTS (in particular, PCDETERMINANTS 246-372) results in enhanced invasion.

### Example 12

### PCDETERMINANTS Directly Drive Invasion In Vitro

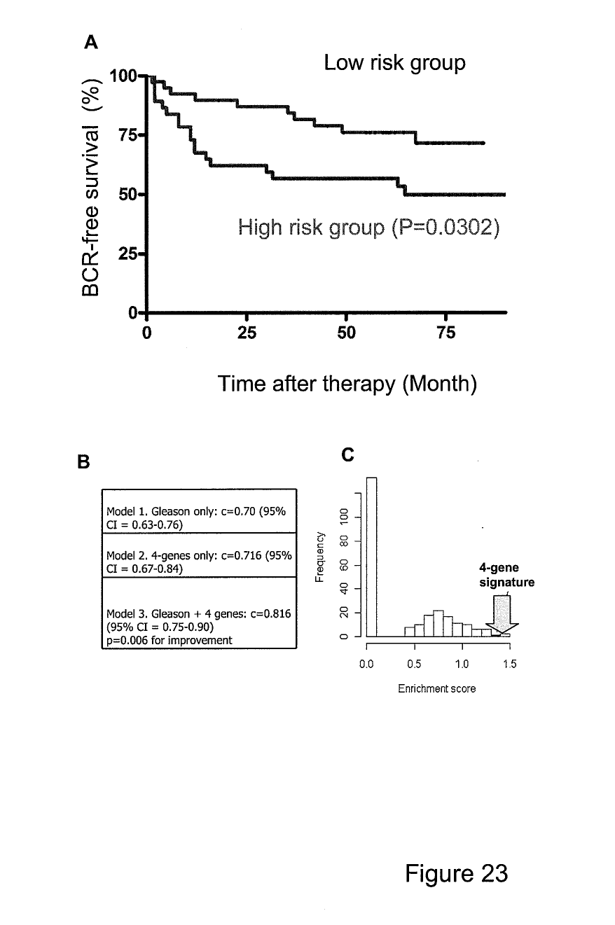

cDNA clones representing up- and down-regulated PCDETERMINANTS were expressed in a pMSCV retroviral system. Human prostate cancer cell line PC3 was individually transduced with retroviral supernatants and assayed in triplicate for invasion using standard 24-well matrigel invasion chambers. Invasiveness of each gene was compared to GFP controls (Table 5). A representative Boyden chamber invasion assay with PC3 cells overexpressing SPP1 and or GFP control in triplicates is shown (FIG. 23A). Enforced expression of SPP1 confirmed its capability to significantly enhance invasive activity of human PCA PC3 cells by invasion assay. The differential level of invasion was statistically significant (P<0.05) (FIG. 23B). Certain invasion-promoting PCDETERMINANTS are annotated as cellular movement genes, whereas others are not (Table 5, FIG. 23C). Interestingly, we found there were 12 hits from those 28 cell movement genes in PC3 cells (43% hits); while there were only 6 hits from 38 genes that were in other functional categories (16% hits). Thus, these functional validation results confirm the veracity of the in silico annotation of the genes are cell movement enabling genes. These functional data documenting pro-invasion activity of putative Smad4-Pten targets, against the backdrop of the in vivo progressive Pten^{pc-/-} Smad4^{pc-/-} tumor phenotype and the in silico cell motility molecular profile, indicate that this invasion block is a major mechanism of progression inhibition by the TGFβ/BMP-Smad4 signaling network, and can be utilized for prioritization of the further clinical validation.

### Example 13

### Small Panels of PCDETERMINANTS are Prognostic in Human Prostate Cancer

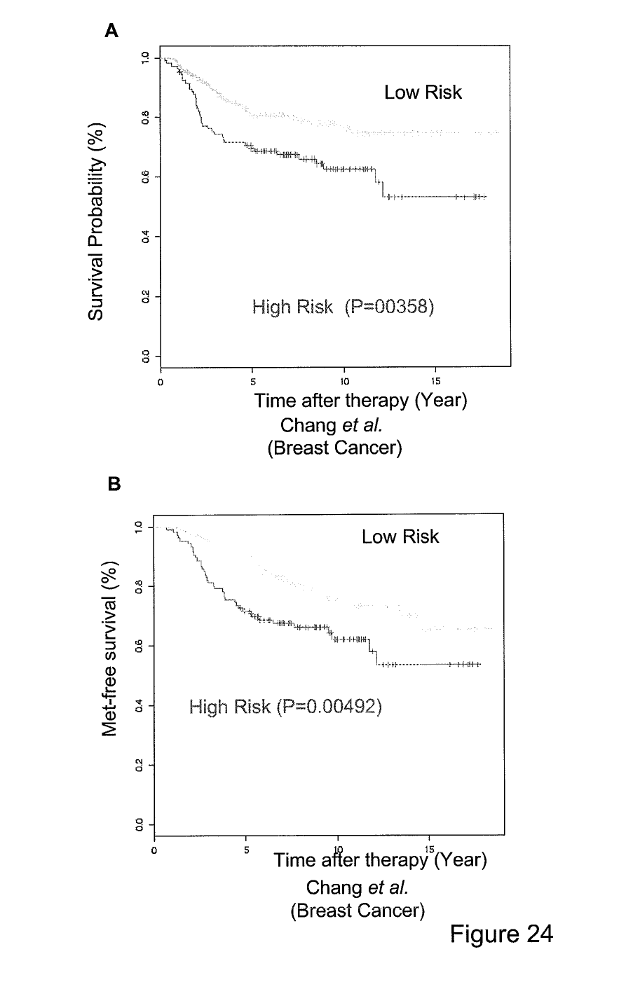

In certain embodiments, it is advantageous to measure 10, 20, 30, 40, 50, 60, 70, 80, 90, 100, 110, 150, 200, 250, 300, 350, or all 372 PCDETERMINANTS to provide prognostic information concerning the propensity of an individual tumor to metastasize. In other embodiments, it is advantageous to leverage small panels PCDETERMINANTS to provide such prognostic information. FIGS. 5, 8, 12, and 21 identify panels comprised of >16 PCDETERMINANTs which stratify human PCA or breast outcome. We next explored the utility of smaller panels of PCDETERMINANTS (FIG. 24). Dysregulated Pten and Smad4 expression together with the related Cyclin D1 (proliferation/senescence) and SPP1 (motility network) was subsequently shown to be correlated with the human prostate cancer progression on a prostate cancer gene expression data set. (FIG. 24A). Patient samples were categorized into two major clusters by K-mean (High-risk and Low risk groups) defined by the PTEN, SMAD4, Cyclin D1, and SPP1 signature. High-risk group patient showed statistically significant in biochemical recurrence (BCR) PSA level (>0.2 ng/ml) by Kaplan-Meier analysis. The significant correlation of PTEN, SMAD4, Cyclin D1, and SPP1 signature in PCA progression was validated in an independent Physicians' Health Study (PHS) dataset with c-statistic. The PTEN, SMAD4, Cyclin D1, and SPP1 show similar power to Gleason score in the prediction of lethal outcomes. The addition of PTEN, SMAD4, Cyclin D1, and SPP1 genes to Gleason significantly improves prediction of lethal outcomes over the model of Gleason alone in PHS (FIG. 24B). Moreover, PTEN, SMAD4, Cyclin D1, and SPP1 4-gene set ranked as the most enriched among 244 bidirectional signatures curated in the Molecular Signature Databases of the Broad Institute (MSigDB, version 2.5), indicating the robust significance of this 4 gene signature in prediction of lethal outcome (FIG. 24C).

### Example 14

### PCDETERMINANTs are Prognostic in Breast

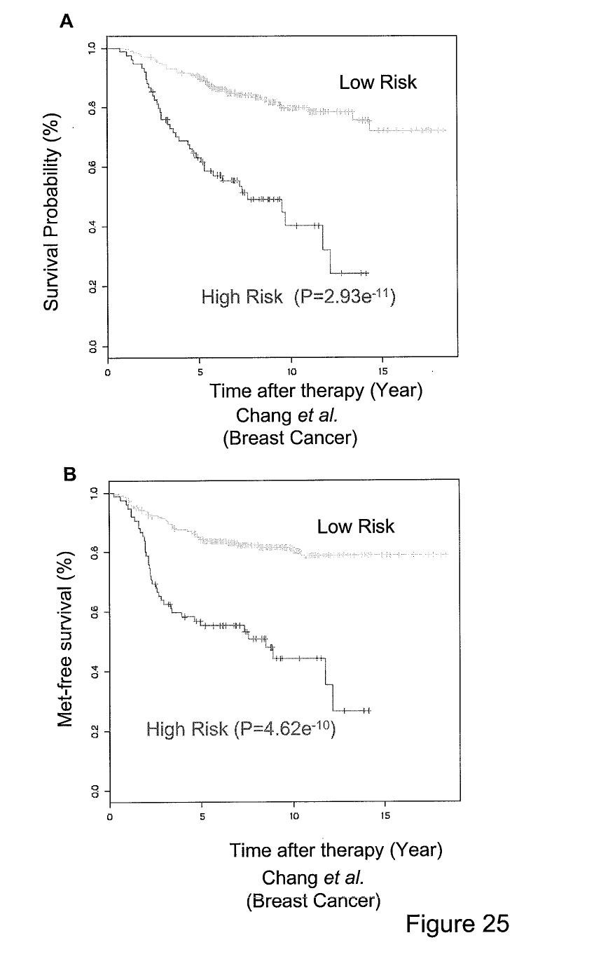

While discovered in the context of prostate cancer, PCDETERMINANTS likely regulate core metastatic processes relevant to multiple cancer types. To explore this possibility, we evaluated the 56 cross-species/cross-platform-filtered PCDETERMINANTS (Table 7) for prognostic utility on a breast adenocarcinoma dataset. Patient samples were categorized into two major clusters (low risk group and high risk group) defined by the 56-gene signature. Kaplan-Meier analysis was conducted for survival probability (p=0.00358) (FIG. 25A) and metastasis-free survival (p=00492) (FIG. 25B) based on the groups defined by the 56-gene cluster. In addition, we next examined the 74 PCDETERMINANTS exhibiting progression correlated expression in prostate cancer (Table 4) and identified 20 PCDETERMINANTS that also exhibit progression-correlated expression in breast cancer. The 20 PCDeterminants exhibiting progression correlated expression in both prostate cancer and breast cancer (Table 6) was evaluated for prognostic utility on a breast adenocarcinoma dataset. Patient samples were categorized into two major clusters (low risk group and high risk group) defined by the 20 progression correlated-gene signature. Kaplan-Meier analysis was conducted for survival probability (p=2.93e⁻¹¹) (FIG. 26A) and metastasis-free survival (p=4.62e⁻¹⁰) (FIG. 26B) based on the groups defined by the 20 PCDeterminants.

TABLE 2
Putative SMAD4 Targets

| Name | Description |
|---|---|
| ARG1 | Arg1: arginase 1, liver |
| ABHD12 | Abhd12: abhydrolase domain containing 12 |
| ALDH1A1 | Aldh1a1: aldehyde dehydrogenase family 1, subfamily A1 |
| CCND2 | Ccnd2: cyclin D2 |
| CD44 | Cd44: CD44 antigen |
| COL12A1 | Col12a1: procollagen, type XII, alpha 1 |
| COL18A1 | Col18a1: procollagen, type XVIII, alpha 1 |
| COL1A1 | Col1a1: procollagen, type I, alpha 1 |
| COL1A2 | Col1a2: procollagen, type I, alpha 2 |
| COL3A1 | Col3a1: procollagen, type III, alpha 1 |
| COL4A1 | Col4a1: procollagen, type IV, alpha 1 |
| COL4A2 | Col4a2: procollagen, type IV, alpha 2 |
| COL5A1 | Col5a1: procollagen, type V, alpha 1 |
| COL5A2 | Col5a2: procollagen, type V, alpha 2 |
| CP | Cp: ceruloplasmin |
| CRLF1 | Crlf1: cytokine receptor-like factor 1 |
| CTSE | Ctse: cathepsin E |
| DEGS2 | Degs2: degenerative spermatocyte homolog 2 |
| | (Drosophila), lipid desaturase |
| FBLN2 | Fbln2: fibulin2 |
| FBN1 | Fbn1: fibrillin 1 |
| FN1 | Fn1: fibronectin 1 |
| FSCN1 | Fscn1: fascin homolog 1, actin bundling protein |
| | (Strongylocentrotus purpuratus) |
| FSTL1 | Fstl1: follistatin-like 1 |
| GJA1 | Gja1: gap junction membrane channel protein alpha 1 |
| GPX2 | Gpx2: glutathione peroxidase 2 |
| GSN | Gsn: gelsolin |
| ID1 | Id1: inhibitor of DNA binding 1 |
| ID3 | Id3: inhibitor of DNA binding 3 |
| IGJ | Igj: immunoglobulin joining chain |
| INHBB | Inhbb: inhibin beta-B |
| KRT14 | Krt14: keratin 14 |
| KRT17 | Krt17: keratin 17 |
| KRT6A | Krt6a: keratin 6A |
| LGALS1 | Lgals1: lectin, galactose binding, soluble 1 |
| LHFP | Lhfp: lipoma HMGIC fusion partner |
| LOX | Lox: lysyl oxidase |
| METTL7A | Mettl7a: methyltransferase like 7A |
| MID1 | Mid1: midline 1 |
| MSN | Msn: moesin |
| NCOA4 | Ncoa4: nuclear receptor coactivator 4 |
| OSMR | Osmr: oncostatin M receptor |
| PLLP | Pllp: plasma membrane proteolipid |
| PLOD2 | Plod2: procollagen lysine, 2-oxoglutarate 5-dioxygenase 2 |
| POSTN | Postn: periostin, osteoblast specific factor |
| PSCA | Psca: prostate stem cell antigen |
| SCNN1A | Scnn1a: sodium channel, nonvoltage-gated, type I, alpha |
| SERPINH1 | Serpinh1: serine (or cysteine) peptidase |
| | inhibitor, clade H, member 1 |
| SFRP1 | Sfrp1: secreted frizzled-related sequence protein 1 |
| SLPI | Slpi: secretory leukocyte peptidase inhibitor |
| SPARC | Sparc: secreted acidic cysteine rich glycoprotein |
| SPON1 | Spon1: spondin 1, (f-spondin) extracellular matrix protein |
| SPP1 | Spp1: secreted phosphoprotein 1 |
| STAT5A | Stat5a: signal transducer and activator of transcription 5 A |
| STEAP4 | Steap4: STEAP family member 4 |
| TESC | Tesc: tescalcin |
| TFF3 | Tff3: trefoil factor 3, intestinal |
| TGFBI | Tgfbi: transforming growth factor, beta induced |
| THBS2 | Thbs2: thrombospondin 2 |
| TIMP1 | Timp1: tissue inhibitor of metalloproteinase 1 |
| TM4SF1 | Tm4sf1: transmembrane 4 superfamily member 1 |
| TMEM45B | Tmem45b: transmembrane protein 45b |
| TNC | Tnc: tenascin C |
| TTR | Ttr: transthyretin |
| UPK1A | Upk1a: uroplakin 1A |
| UPK1B | Upk1b: uroplakin 1B |
| ZBTB16 | Zbtb16: zinc finger and BTB domain containing 16 |

TABLE 3
This represents the 17 SMAD4 targets

| Name | Description |
|---|---|
| ALDH1A1 | Aldh1a1: aldehyde dehydrogenase family 1, subfamily A1 |
| CP | Cp: ceruloplasmin |
| FBN1 | Fbn1: fibrillin 1 |
| FSCN1 | Fscn1: fascin homolog 1, actin bundling protein |
| | (Strongylocentrotus purpuratus) |
| GPX2 | Gpx2: glutathione peroxidase 2 |
| ID3 | Id3: inhibitor of DNA binding 3 |
| KRT14 | Krt14: keratin 14 |
| KRT17 | Krt17: keratin 17 |
| KRT6A | Krt6a: keratin 6A |
| LHFP | Lhfp: lipoma HMGIC fusion partner |
| OSMR | Osmr: oncostatin M receptor |
| PLOD2 | Plod2: procollagen lysine, 2-oxoglutarate 5-dioxygenase 2 |
| PSCA | Psca: prostate stem cell antigen |
| SPP1 | Spp1: secreted phosphoprotein 1 |
| TM4SF1 | Tm4sf1: transmembrane 4 superfamily member 1 |
| UPK1B | Upk1b: uroplakin 1B |
| ZBTB16 | Zbtb16: zinc finger and BTB domain containing 16 |

TABLE 4
PCDETERMINANTS exhiting Progression-Correlated Expression
patterns in prostate cancer within the Oncomine database

| | |
|---|---|
| Name | Description |

Up-Regulated Genes

| | |
|---|---|
| ADAM8 | Adam8: a disintegrin and metallopeptidase domain 8 |
| AK1 | Ak1: adenylate kinase 1 |
| ANGPTL4 | Angptl4: angiopoietin-like 4 |
| B4GALT5 | B4galt5: UDP-Gal: betaGlcNAc beta |
| | 1,4-galactosyltransferase, polypeptide 5 |
| BIRC5 | Birc5: baculoviral IAP repeat-containing 5 |
| BST1 | bone marrow stromal cell antigen 1 |
| CCND1 | Ccnd1: cyclin D1 |
| CDC2 | Cdc2a: cell division cycle 2 homolog A (S. pombe) |
| CDCA8 | Cdca8: cell division cycle associated 8 |
| CENPA | Cenpa: centromere protein A |
| COL18A1 | Col18a1: procollagen, type XVIII, alpha 1 |
| COL1A1 | Col1a1: procollagen, type I, alpha 1 |
| COL3A1 | Col3a1: procollagen, type III, alpha 1 |
| COL5A2 | Col5a2: procollagen, type V, alpha 2 |
| ETS1 | Ets1: E26 avian leukemia oncogene 1, 5′ domain |
| FSCN1 | Fscn1: fascin homolog 1, actin bundling protein |
| | (Strongylocentrotus purpuratus) |
| HMGB2 | high-mobility group box 2 |
| ITGB2 | Itgb2: integrin beta 2 |
| KIAA0101 | 2810417H13Rik: RIKEN cDNA 2810417H13 gene |
| KLK7 | kallikrein-related peptidase 7 |
| KRT6A | Krt6a: keratin 6A |
| LAMB1 | Lamb1-1: laminin B1 subunit 1 |
| LRIG1 | leucine-rich repeats and immunoglobulin-like domains 1 |
| MCM5 | Mcm5: minichromosome maintenance deficient 5, cell |
| | division cycle 46 (S. cerevisiae) |
| MKI67 | antigen identified by monoclonal antibody Ki-67 |
| NCF4 | Ncf4: neutrophil cytosolic factor 4 |
| OLFML2B | Olfml2b: olfactomedin-like 2B |
| PDPN | Pdpn: podoplanin |
| PLOD2 | Plod2: procollagen lysine, 2-oxoglutarate 5-dioxygenase 2 |
| SLC16A1 | Slc16a1: solute carrier family 16 (monocarboxylic acid |
| | transporters), member 1 |
| SPI1 | Sfpi1: SFFV proviral integration 1 |
| SPP1 | Spp1: secreted phosphoprotein 1 |
| STEAP3 | STEAP family member 3 |
| THBS2 | Thbs2: thrombospondin 2 |
| TNFRSF12A | Tnfrsf12a: tumor necrosis factor receptor superfamily, |
| | member 12a |
| TOP2A | Top2a: topoisomerase (DNA) II alpha |
| UBE2C | Ube2c: ubiquitin-conjugating enzyme E2C |
| VCAN | Versican |

Down-Regulated Genes

| | |
|---|---|
| ALDH1A1 | Aldh1a1: aldehyde dehydrogenase family 1, subfamily A1 |
| ATRN | Atrn: attractin |
| BEX4 | brain expressed, X-linked 4 |
| CYB5B | Cyb5b: cytochrome b5 type B |
| FMOD | fibromodulin |
| GSN | Gsn: gelsolin |
| GSTM5 | glutathione S-transferase mu 5 |
| GSTO1 | Gsto1: glutathione S-transferase omega 1 |
| ID1 | Id1: inhibitor of DNA binding 1 |
| ID2 | Id2: inhibitor of DNA binding 2 |
| IQGAP2 | Iqgap2: IQ motif containing GTPase activating protein 2 |
| KRT15 | Krt15: keratin 15 |
| LASS4 | LAG1 homolog, ceramide synthase 4 |
| METTL7A | Mettl7a: methyltransferase like 7A |
| MID1 | Mid1: midline 1 |
| MSMB | microseminoprotein, beta- |
| NCOA4 | Ncoa4: nuclear receptor coactivator 4 |
| ONECUT2 | one cut homeobox 2 |
| PEX1 | peroxisomal biogenesis factor 1 |
| PINK1 | Pink1: PTEN induced putative kinase 1 |
| PTEN | phosphatase and tensin homolog |
| PTGS1 | Ptgs1: prostaglandin-endoperoxide synthase 1 |
| RAB27B | Rab27b: RAB27b, member RAS oncogene family |
| SATB1 | SATB homeobox 1 |
| SCNN1A | Scnn1a: sodium channel, nonvoltage-gated, type 1, alpha |
| SLC25A26 | solute carrier family 25, member 26 |
| SMAD4 | SMAD family member 4 |
| SPINT1 | serine peptidase inhibitor, Kunitz type 1 |
| STAT5A | Stat5a: signal transducer and activator of transcription 5A |
| SUOX | sulfite oxidase |
| TBX3 | Tbx3: T-box 3 |
| TFF3 | Tff3: trefoil factor 3, intestinal |
| TGM4 | transglutaminase 4 (prostate) |
| TMEM45B | Tmem45b: transmembrane protein 45b |
| TRIM2 | Trim2: tripartite motif protein 2 |
| UPK1A | Upk1a: uroplakin 1A |

TABLE 5
PCDETERMINANTS that functionally impact invasion in vitro

| | | Result | |
|---|---|---|---|
| | | (Fold | |
| Name | Description | change) | Annotation |
| GSN | Gsn: gelsolin | 0.1 | Cell Movement |
| ID4 | Id4: inhibitor of DNA binding 4 | 0.1 | Other |
| ID1 | Id1: inhibitor of DNA binding 1 | 0.2 | Cell Movement |
| ZBTB16 | Zbtb16: zinc finger and BTB | 0.2 | Cell Movement |
| | domain containing 16 | | |
| PINK1 | Pink1: PTEN induced putative | 0.4 | Other |
| | kinase 1 | | |
| TTR | Ttr: transthyretin | 0.4 | Other |
| UGT2B15 | Ugt2b35: UDP glucuronosyl- | 0.4 | Other |
| | transferase 2 family, | | |
| | polypeptide B35 | | |
| CTSE | Ctse: cathepsin E | 0.5 | Cell Movement |
| MID1 | Mid1: midline 1 | 0.5 | Other |
| CD53 | Cd53: CD53 antigen | 1.8 | Cell Movement |
| SLPI | Slpi: secretory leukocyte | 2.2 | Cell Movement |
| | peptidase inhibitor | | |
| CD44VE | Cd44VE: CD44 antigen isoform | 2.4 | Cell Movement |
| | contains eight out of the ten | | |
| | variable CD44 exons (v3-v10) | | |
| LOX | Lox: lysyl oxidase | 2.6 | Cell Movement |
| TM4SF1 | Tm4sf1: transmembrane 4 | 2.64 | Other |
| | superfamily member 1 | | |
| FSCN1 | Fscn1: fascin homolog 1, actin | 3.1 | Cell Movement |
| | bundling protein | | |
| | (Strongylocentrotus | | |
| | purpuratus) | | |
| LGALS1 | Lgals1: lectin, galactose | 3.3 | Cell Movement |
| | binding, soluble 1 | | |
| SPP1 | Spp1: secreted phosphoprotein 1 | 3.3 | Cell Movement |
| KRT6A | Krt6a: keratin 6A | 6.5 | Cell Movement |
| ABHD12 | Abhd12: abhydrolase domain | Not Hit | Other |
| | containing 12 | | |
| ADAM19 | Adam19: a disintegrin and | Not Hit | Other |
| | metallopeptidase domain 19 | | |
| | (meltrin beta) | | |
| ALDH1A1 | Aldh1a1: aldehyde dehydro- | Not Hit | Other |
| | genase family 1, subfamily A1 | | |
| ARG1 | Arg1: arginase 1, liver | Not Hit | Other |
| BIRC5 | Birc5: baculoviral IAP | Not Hit | Other |
| | repeat-containing 5 | | |
| C4orf18 | 1110032E23Rik: RIKEN cDNA | Not Hit | Other |
| | 1110032E23 gene | | |
| CCND2 | Ccnd2: cyclin D2 | Not Hit | Other |
| CDCA8 | Cdca8: cell division cycle | Not Hit | Other |
| | associated 8 | | |
| COL3A1 | Col3a1: procollagen, type III, | Not Hit | Other |
| | alpha 1 | | |
| DDAH1 | Ddah1: dimethylarginine | Not Hit | Other |
| | dimethylaminohydrolase 1 | | |
| FKBP10 | Fkbp10: FK506 binding | Not Hit | Other |
| | protein 10 | | |
| FSTL1 | Fstl1: follistatin-like 1 | Not Hit | Cell Movement |
| GJA1 | Gja1: gap junction membrane | Not Hit | Cell Movement |
| | channel protein alpha 1 | | |
| ID3 | Id3: inhibitor of DNA | Not Hit | Cell Movement |
| | binding 3 | | |
| IGF1 | Igf1: insulin-like growth | Not Hit | Cell Movement |
| | factor 1 | | |
| IL4R | Il4ra: interleukin 4 receptor, | Not Hit | Cell Movement |
| | alpha | | |
| INHBB | Inhbb: inhibin beta-B | Not Hit | Other |
| ITGAX | Itgax: integrin alpha X | Not Hit | Cell Movement |
| ITGB2 | Itgb2: integrin beta 2 | Not Hit | Cell Movement |
| JUB | Jub: ajuba | Not Hit | Cell Movement |
| KRT14 | Krt14: keratin 14 | Not Hit | Other |
| KRT17 | Krt17: keratin 17 | Not Hit | Other |
| LGALS7 | Lgals7: lectin, galactose | Not Hit | Other |
| | binding, soluble 7 | | |
| LHFP | Lhfp: lipoma HMGIC fusion | Not Hit | Other |
| | partner | | |
| LOXL2 | Loxl2: lysyl oxidase-like 2 | Not Hit | Other |
| METTL7A | Mettl7a: methyltransferase | Not Hit | Other |
| | like 7A | | |
| MSN | Msn: moesin | Not Hit | Cell Movement |
| NCOA4 | Ncoa4: nuclear receptor | Not Hit | Cell Movement |
| | coactivator 4 | | |
| OLFML2B | Olfml2b: olfactomedin-like 2B | Not Hit | Other |
| OSMR | Osmr: oncostatin M receptor | Not Hit | Other |
| PLLP | Pllp: plasma membrane | Not Hit | Other |
| | proteolipid | | |
| PLOD2 | Plod2: procollagen lysine, 2- | Not Hit | Other |
| | oxoglutarate 5-dioxygenase 2 | | |
| PSCA | Psca: prostate stem | Not Hit | Other |
| | cell antigen | | |
| PTGS1 | Ptgs1: prostaglandin- | Not Hit | Other |
| | endoperoxide synthase 1 | | |
| PXDN | Pxdn: peroxidasin homolog | Not Hit | Other |
| | (Drosophila) | | |
| SERPINH1 | Serpinh1: serine (or cysteine) | Not Hit | Other |
| | peptidase inhibitor, clade H, | | |
| | member 1 | | |
| SH3PXD2B | Sh3pxd2b: SH3 and PX | Not Hit | Other |
| | domains 2B | | |
| SPARC | Sparc: secreted acidic | Not Hit | Cell Movement |
| | cysteine rich glycoprotein | | |
| SPI1 | Sfpi1: SFFV proviral | Not Hit | Cell Movement |
| | integration 1 | | |
| SPON1 | Spon1: spondin 1, (f-spondin) | Not Hit | Other |
| | extracellular matrix protein | | |
| SPRR2G | Sprr2a: small proline-rich | Not Hit | Other |
| | protein 2A | | |
| STAT5A | Stat5a: signal transducer and | Not Hit | Cell Movement |
| | activator of transcription 5A | | |
| TESC | Tesc: tescalcin | Not Hit | Other |
| TFF3 | Tff3: trefoil factor 3, | Not Hit | Cell Movement |
| | intestinal | | |
| TGFBI | Tgfbi: transforming growth | Not Hit | Cell Movement |
| | factor, beta induced | | |
| TIMP1 | Timp1: tissue inhibitor of | Not Hit | Cell Movement |
| | metalloproteinase 1 | | |
| TMEM45B | Tmem45b: transmembrane | Not Hit | Other |
| | protein 45b | | |
| UPK1B | Upk1b: uroplakin 1B | Not Hit | Other |

TABLE 6
PCDETERMINANTS Exhibiting Progression Correlated Expression
in Both Human Prostate and Breast Cancers

| Name | Description |
|---|---|
| ADAM8 | Adam8: a disintegrin and metallopeptidase domain 8 |
| ANGPTL4 | Angptl4: angiopoietin-like 4 |
| BIRC5 | Birc5: baculoviral IAP repeat-containing 5 |
| CCND1 | Ccnd1: cyclin D1 |
| CDC2 | Cdc2a: cell division cycle 2 homolog A (S. pombe) |
| CDCA8 | Cdca8: cell division cycle associated 8 |
| CENPA | Cenpa: centromere protein A |
| KIAA0101 | 2810417H13Rik: RIKEN cDNA 2810417H13 gene |
| MCM5 | Mcm5: minichromosome maintenance deficient 5, cell |
| | division cycle 46 (S. cerevisiae) |
| PLOD2 | Plod2: procollagen lysine, 2-oxoglutarate 5-dioxygenase 2 |
| SLC16A1 | Slc16a1: solute carrier family 16 (monocarboxylic |
| | acid transporters), member 1 |
| SPP1 | Spp1: secreted phosphoprotein 1 |
| TOP2A | Top2a: topoisomerase (DNA) II alpha |
| UBE2C | Ube2c: ubiquitin-conjugating enzyme E2C |
| MKI67 | antigen identified by monoclonal antibody Ki-67 |
| SMAD4 | SMAD family member 4 |
| TFF3 | Tff3: trefoil factor 3, intestinal |
| PTEN | phosphatase and tensin homolog |
| FMOD | fibromodulin |
| SUOX | sulfite oxidase |

TABLE 7
56 PCDETERMINANTS with Altered DNA Copy Number Alterations
in Human Metastatic PCA a CGH dataset

| | |
|---|---|
| Name | Description |

Up-Regulated Genes

| | |
|---|---|
| ADAM19 | Adam19: a disintegrin and metallopeptidase domain 19 |
| | (meltrin beta) |
| ANTXR2 | Antxr2: anthrax toxin receptor 2 |
| C1QB | C1qb: complement component 1, q subcomponent, beta |
| | polypeptide |
| CD200 | Cd200: Cd200 antigen |
| CD248 | Cd248: CD248 antigen, endosialin |
| COL8A1 | Col8a1: procollagen, type VIII, alpha 1 |
| CP | Cp: ceruloplasmin |
| FBN1 | Fbn1: fibrillin 1 |
| FKBP10 | Fkbp10: FK506 binding protein 10 |
| FRZB | Frzb: frizzled-related protein |
| FSCN1 | Fscn1: fascin homolog 1, actin bundling protein |
| | (Strongylocentrotus purpuratus) |
| GCNT2 | glucosaminyl (N-acetyl) transferase 2, I-branching |
| | enzyme (I blood group) |
| GPX2 | Gpx2: glutathione peroxidase 2 |
| HPR | Hp: haptoglobin |
| JAG1 | Jag1: jagged 1 |
| KLHL6 | kelch-like 6 (Drosophila) |
| KRT14 | Krt14: keratin 14 |
| KRT17 | Krt17: keratin 17 |
| KRT5 | Krt5: keratin 5 |
| KRT6A | Krt6a: keratin 6A |
| LGMN | Lgmn: legumain |
| LHFP | Lhfp: lipoma HMGIC fusion partner |
| MKI67 | antigen identified by monoclonal antibody Ki-67 |
| MSRB3 | Msrb3: methionine sulfoxide reductase B3 |
| NID1 | Nid1: nidogen 1 |
| OSMR | Osmr: oncostatin M receptor |
| PDPN | Pdpn: podoplanin |
| PLA2G7 | Pla2g7: phospholipase A2, group VII (platelet- |
| | activating factor acetylhydrolase, plasma) |
| PLOD2 | Plod2: procollagen lysine, 2-oxoglutarate |
| | 5-dioxygenase 2 |
| PPIC | Ppic: peptidylprolyl isomerase C |
| RBP1 | Rbp1: retinol binding protein 1, cellular |
| RGS4 | Rgs4: regulator of G-protein signaling 4 |
| SPP1 | Spp1: secreted phosphoprotein 1 |
| TM4SF1 | Tm4sf1: transmembrane 4 superfamily member 1 |
| TOP2A | Top2a: topoisomerase (DNA) II alpha |
| WISP1 | WNT1 inducible signaling pathway protein 1 |

Down-Regulated Genes

| | |
|---|---|
| ALDH1A1 | Aldh1a1: aldehyde dehydrogenase family 1, subfamily A1 |
| ARHGEF4 | Arhgef4: Rho guanine nucleotide exchange factor (GEF) 4 |
| EPS8L3 | EPS8-like 3 |
| GPLD1 | Gpld1: glycosylphosphatidylinositol specific |
| | phospholipase D1 |
| HSPC105 | 4632417N05Rik: RIKEN cDNA 4632417N05 gene |
| ID3 | Id3: inhibitor of DNA binding 3 |
| KBTBD11 | Kbtbd11: kelch repeat and BTB (POZ) domain |
| | containing 11 |
| KRT4 | Krt4: keratin 4 |
| LY6K | lymphocyte antigen 6 complex, locus K |
| M-RIP | AA536749: Expressed sequence AA536749 |
| PAPSS2 | Papss2: 3′-phosphoadenosine 5′-phosphosulfate |
| | synthase 2 |
| PEX1 | peroxisomal biogenesis factor 1 |
| PITX2 | paired-like homeodomain 2 |
| PSCA | Psca: prostate stem cell antigen |
| PTEN | phosphatase and tensin homolog |
| SLC16A7 | solute carrier family 16, member 7 (monocarboxylic |
| | acid transporter 2) |
| TMEM56 | transmembrane protein 56 |
| UPK1B | Upk1b: uroplakin 1B |
| ZBTB16 | Zbtb16: zinc finger and BTB domain containing 16 |
| ZDHHC14 | Zdhhc14: zinc finger, DHHC domain containing 14 |

### REFERENCES

### Reference List

1. Jemal, A. et al. Cancer statistics, 2008. CA Cancer J. Clin. 58, 71-96 (2008).
2. Walsh, P. C., DeWeese, T. L. & Eisenberger, M. A. Clinical practice. Localized prostate cancer. N. Engl. J. Med. 357, 2696-2705 (2007).
3. Li, J. et al. PTEN, a putative protein tyrosine phosphatase gene mutated in human brain, breast, and prostate cancer. Science 275, 1943-1947 (1997).
4. Tomlins, S. A. et al. Recurrent fusion of TMPRSS2 and ETS transcription factor genes in prostate cancer. Science 310, 644-648 (2005).
5. Rubin, M. A. Targeted therapy of cancer: new roles for pathologists—prostate cancer. Mod. Pathol. 21 Suppl 2, S44-S55 (2008).
6. Abate-Shen, C., Shen, M. M. & Gelmann, E. Integrating differentiation and cancer: The Nkx3.1 homeobox gene in prostate organogenesis and carcinogenesis. Differentiation (2008).
7. Tomlins, S. A. et al. The role of SPINK1 in ETS rearrangement-negative prostate cancers. Cancer Cell 13, 519-528 (2008).
8. Jenkins, R. B., Qian, J., Lieber, M. M. & Bostwick, D. G. Detection of c-myc oncogene amplification and chromosomal anomalies in metastatic prostatic carcinoma by fluorescence in situ hybridization. Cancer Res. 57, 524-531 (1997).
9. Rubin, M. A. et al. E-cadherin expression in prostate cancer: a broad survey using high-density tissue microarray technology. Hum. Pathol. 32, 690-697 (2001).
10. Chaib, H. et al. Activated in prostate cancer: a PDZ domain-containing protein highly expressed in human primary prostate tumors. Cancer Res. 61, 2390-2394 (2001).
11. Dhanasekaran, S. M. et al. Delineation of prognostic biomarkers in prostate cancer. Nature 412, 822-826 (2001).
12. Rubin, M. A. et al. alpha-Methylacyl coenzyme A racemase as a tissue biomarker for prostate cancer. JAMA 287, 1662-1670 (2002).
13. Rhodes, D. R., Sanda, M. G., Otte, A. P., Chinnaiyan, A. M. & Rubin, M. A. Multiplex biomarker approach for determining risk of prostate-specific antigen-defined recurrence of prostate cancer. J. Natl. Cancer Inst. 95, 661-668 (2003).
14. Varambally, S. et al. The polycomb group protein EZH2 is involved in progression of prostate cancer. Nature 419, 624-629 (2002).
15. Glinsky, G. V., Glinskii, A. B., Stephenson, A. J., Hoffman, R. M. & Gerald, W. L. Gene expression profiling predicts clinical outcome of prostate cancer. J. Clin. Invest 113, 913-923 (2004).
16. Varambally, S. et al. Integrative genomic and proteomic analysis of prostate cancer reveals signatures of metastatic progression. Cancer Cell 8, 393-406 (2005).
17. Tomlins, S. A. et al. Integrative molecular concept modeling of prostate cancer progression. Nat. Genet. 39, 41-51 (2007).
18. Yu, Y. P. et al. Gene expression alterations in prostate cancer predicting tumor aggression and preceding development of malignancy. J. Clin. Oncol. 22, 2790-2799 (2004).
19. Kim, J. H. et al. Integrative analysis of genomic aberrations associated with prostate cancer progression. Cancer Res. 67, 8229-8239 (2007).
20. Chang, H. Y. et al. Robustness, scalability, and integration of a wound-response gene expression signature in predicting breast cancer survival. Proc. Natl. Acad. Sci. U.S.A 102, 3738-3743 (2005).
21. Kim, M. et al. Comparative oncogenomics identifies NEDD9 as a melanoma metastasis gene. Cell 125, 1269-1281 (2006).
22. Sweet-Cordero, A. et al. An oncogenic KRAS2 expression signature identified by cross-species gene-expression analysis. Nat. Genet. 37, 48-55 (2005).
23. Zender, L. et al. Identification and validation of oncogenes in liver cancer using an integrative oncogenomic approach. Cell 125, 1253-1267 (2006).
24. Maser, R. S. et al. Chromosomally unstable mouse tumours have genomic alterations similar to diverse human cancers. Nature 447, 966-971 (2007).
25. Faca, V. M. et al. A mouse to human search for plasma proteome changes associated with pancreatic tumor development. PLoS. Med. 5, e123 (2008).
26. Chen, Z. et al. Crucial role of p53-dependent cellular senescence in suppression of Pten-deficient tumorigenesis. Nature 436, 725-730 (2005).
27. Wang, S. et al. Prostate-specific deletion of the murine Pten tumor suppressor gene leads to metastatic prostate cancer. Cancer Cell 4, 209-221 (2003).
28. Massague, J., Seoane, J. & Wotton, D. Smad transcription factors. Genes Dev. 19, 2783-2810 (2005).
29. Lee, C. et al. Transforming growth factor-beta in benign and malignant prostate. Prostate 39, 285-290 (1999).
30. Pardali, K. & Moustakas, A. Actions of TGF-beta as tumor suppressor and pro-metastatic factor in human cancer. Biochim. Biophys. Acta 1775, 21-62 (2007).
31. Bierie, B. & Moses, H. L. Tumour microenvironment: TGFbeta: the molecular Jekyll and Hyde of cancer. Nat. Rev. Cancer 6, 506-520 (2006).
32. Bardeesy, N. et al. Smad4 is dispensable for normal pancreas development yet critical in progression and tumor biology of pancreas cancer. Genes Dev. 20, 3130-3146 (2006).
33. Ao, M., Williams, K., Bhowmick, N. A. & Hayward, S. W. Transforming growth factor-beta promotes invasion in tumorigenic but not in nontumorigenic human prostatic epithelial cells. Cancer Res. 66, 8007-8016 (2006).
34. Zavadil, J. & Bottinger, E. P. TGF-beta and epithelial-to-mesenchymal transitions. Oncogene 24, 5764-5774 (2005).
35. Padua, D. et al. TGFbeta primes breast tumors for lung metastasis seeding through angiopoietin-like 4. Cell 133, 66-77 (2008).
36. Zheng, H. et al. Cooperative actions of p53 and Pten in normal and neoplastic stem/progenitor cell differentiation and in primary glioblastoma. Nature Submitted., (2008).
37. Wu, X. et al. Generation of a prostate epithelial cell-specific Cre transgenic mouse model for tissue-specific gene ablation. Mech. Dev. 101, 61-69 (2001).
38. Watson, P. A. et al. Context-dependent hormone-refractory progression revealed through characterization of a novel murine prostate cancer cell line. Cancer Res. 65, 11565-11571 (2005).
39. Irizarry, R. A. et al. Summaries of Affymetrix GeneChip probe level data. Nucleic Acids Res. 31, e15 (2003).
40. Gentleman, R. C. et al. Bioconductor: open software development for computational biology and bioinformatics. Genome Biol. 5, R80 (2004).
41. Tusher, V. G., Tibshirani, R. & Chu, G. Significance analysis of microarrays applied to the ionizing radiation response. Proc. Natl. Acad. Sci. U. S. A 98, 5116-5121 (2001).
42. Matys, V. et al. TRANSFAC: transcriptional regulation, from patterns to profiles. Nucleic Acids Res. 31, 374-378 (2003).
43. Lenhard, B. & Wasserman, W. W. TFBS: Computational framework for transcription factor binding site analysis. Bioinformatics. 18, 1135-1136 (2002).
44. Birney, E. et al. Ensembl 2006. Nucleic Acids Res. 34, D556-D561 (2006).
45. Ho Sui, S. J. et al. oPOSSUM: identification of over-represented transcription factor binding sites in co-expressed genes. Nucleic Acids Res. 33, 3154-3164 (2005).
46. Khoo, C. M., Carrasco, D. R., Bosenberg, M. W., Paik, J. H. & DePinho, R. A. Ink4a/Arf tumor suppressor does not modulate the degenerative conditions or tumor spectrum of the telomerase-deficient mouse. Proc. Natl. Acad. Sci. U.S. A 104, 3931-3936 (2007).
47. Trotman, L. C. et al. Pten Dose Dictates Cancer Progression in the Prostate. PLoS. Biol. 1, E59 (2003).

We claim:

1. A method with a predetermined level of predictability for assessing a risk of cancer
recurrence or development of a metastatic cancer in a subject comprising:
a. measuring the level of two or more PCDETERMINANTS selected from the group consisting of PCDETERMINANTS 1-372 in a sample from the subject, and
b. measuring a clinically significant alteration in the level of the two or more PCDETERMINANTS in the sample, wherein the alteration indicates an increased risk of cancer recurrence or developing metastatic cancer in the subject.

2. The method of claim 1, wherein said two or more PCDETERMINANTS are selected from
a) Table 2;
b) Table 3;
c) Table 4;
d) Table 5;
e) Table 6;
f) Table 7; and
g) two or more Tables selected from Tables 2-7.

3. The method of claim 1, wherein said two or more PCDETERMINANTS include two or more of PTEN, SMAD4, cyclin D1 and SPP1.

4. The method of claim 1, further comprising measuring at least one standard parameter associated with said cancer.

5. The method of claim 4, wherein said cancer is a prostate cancer and said standard parameter is Gleason score.

6. The method of claim 1, wherein the level of a PCDETERMINANT is measured electrophoretically, immunochemically or by non-invasive imaging.

7. The method of claim 6, wherein the immunochemical detection is by radioimmunoassay, immunofluorescence assay or by an enzyme-linked immunosorbent assay.

8. The method of claim 1, wherein the subject has a primary tumor, a recurrent tumor, or metastatic prostate cancer.

9. The method of claim 1, wherein the sample is a tumor biopsy, blood, or a circulating tumor cell in a biological fluid.

10. The method of claim 1, wherein said biopsy is a core biopsy, an excisional tissue biopsy or an incisional tissue biopsy.

11-13. (canceled)

14. A method with a predetermined level of predictability for assessing the progression of a tumor in a subject, for monitoring the effectiveness of a treatment for a recurrent or a metastatic cancer in a subject, or for selecting a treatment regimen for a subject diagnosed with a tumor, the method comprising:
a. detecting the level of two or more PCDETERMINANTS selected from the group consisting of PCDETERMINANTS 1-372 in a first sample from the subject at a first period of time;
b. detecting the level of two or more PCDETERMINANTS in a second sample from the subject at a second period of time;
c. comparing the level of the two or more PCDETERMINANTS detected in step (a) to the level detected in step (b), or to a reference value.

15-26. (canceled)

27. A metastatic prostate cancer reference expression profile, comprising a pattern of marker levels of two or more markers selected from the group consisting of PCDETERMINANTS 1-372.

28. A kit comprising a plurality of PCDETERMINANT detection reagents that detect the corresponding PCDETERMINANTS selected from the group consisting of PCDETERMINANTS 1-372, sufficient to generate the profile of claim 27.

29-32. (canceled)

33. A PCDETERMINANT panel comprising one or more PCDETERMINANTS that are indicative of a physiological or biochemical pathway associated metastasis or that are indicative of the progression of a tumor.

34-57. (canceled)

58. The method of claim 3, wherein said two or more PCDETERMINANTS include three or more of PTEN, SMAD4, cyclin D1 and SPP1.

59. The method of claim 58, wherein said two or more PCDETERMINANTS include PTEN, SMAD4, cyclin D1 and SPP1.


SEQUENCE LISTING (6 sequences)

SEQ ID NO: 1 - DNA, 23 bp, Artificial Sequence
  Description of Artificial Sequence Synthetic primer
cttcggagcatgtctggcaatgc

SEQ ID NO: 2 - DNA, 24 bp, Artificial Sequence
  Description of Artificial Sequence Synthetic primer
ctgcacgagactagtgagacgtgc

SEQ ID NO: 3 - DNA, 20 bp, Artificial Sequence
  Description of Artificial Sequence Synthetic primer
aaggaagagggtggggatac

SEQ ID NO: 4 - DNA, 28 bp, Artificial Sequence
  Description of Artificial Sequence Synthetic primer
gggaacagagcacaggcctctgtgacag

SEQ ID NO: 5 - DNA, 28 bp, Artificial Sequence
  Description of Artificial Sequence Synthetic primer
ttcactgtgtagccccgcctgtcctgga

SEQ ID NO: 6 - DNA, 23 bp, Artificial Sequence
  Description of Artificial Sequence Synthetic primer
tgctctgagctcacaattctcct